SEQUENCE LISTING (115 sequences)

SEQ ID NO: 1 - DNA, 318 bp, Solanum tuberosum
agacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggc

SEQ ID NO: 2 - DNA, 961 bp, Zea mays
gatccgattgactatctcattcctccaaacccaaacacctcaaatatatctgctatcgggattggcattcctgtatccctacgcccgtgtaccccctgtttagagaacctcccaaggtataagatggcgaagattattgttgtcttgtctttcatcatatatcgagtctttccctaggatattattattggcaatgagcattacacggttaatcgattgagagaacatgcatctcaccttcagcaaataattacgataatccatattttacgcttcgtaacttctcatgagtttcgatatacaaatttgttttctggacaccctaccattcatcctcttcggagaagagaggaagtgtcctcaatttaaatatgttgtcatgctgtagttcttcacccaatctcaacaggtaccaagcacattgtttccacaaattatattttagtcacaataaatctatattattattaatatactaaaactatactgacgctcagatgcttttactagttcttgctagtatgtgatgtaggtctacgtggaccagaaaatagtgagacacggaagacaaaagaagtaaaagaggcccggactacggcccacatgagattcggccccgccacctccggcaaccagcggccgatccaacggaagtgcgcgcacacacacaacctcgtatatatcgccgcgcggaagcggcgcgaccgaggaagccttgtcctcgacaccccctacacaggtgtcgcgctgcccccgacacgagtcccgcatgcgtcccacgcggccgcgccagatcccgcctccgcgcgttgccacgccctctataaacacccagctctccctcgccctcatctacctcactcgtagtcgtagctcaagcatcagcggcagcggcagcggcaggagctctgggcagcgtgcgcacgtggggtacctagctcgctctgctagcctaccttaa

SEQ ID NO: 3 - DNA, 8 bp, Artificial Sequence
  Synthesized
yacgtggc

SEQ ID NO: 4 - DNA, 9 bp, Artificial Sequence
  Synthesized
tgccaccgg

SEQ ID NO: 5 - DNA, 11 bp, Artificial Sequence
  Synthesized
acgcgtgcctc

SEQ ID NO: 6 - DNA, 7 bp, Artificial Sequence
  Synthesized
acgtgtc

SEQ ID NO: 7 - DNA, 7 bp, Artificial Sequence
  Synthesized
acgtggc

SEQ ID NO: 8 - DNA, 7 bp, Artificial Sequence
  Synthesized
acgtgtc

SEQ ID NO: 9 - DNA, 7 bp, Artificial Sequence
  Synthesized
tggttag

SEQ ID NO: 10 - DNA, 6 bp, Artificial Sequence
  Synthesized
cacatg

SEQ ID NO: 11 - DNA, 9 bp, Artificial Sequence
  Synthesized
taccgacat

SEQ ID NO: 12 - DNA, 9 bp, Artificial Sequence
  Synthesized
ggccgacat

SEQ ID NO: 13 - DNA, 9 bp, Artificial Sequence
  Synthesized
ggccgacgt

SEQ ID NO: 14 - DNA, 10 bp, Artificial Sequence
  Synthesized
acacgcatgt

SEQ ID NO: 15 - DNA, 14 bp, Artificial Sequence
  Synthesized
ggacacatgtcaga

SEQ ID NO: 16 - DNA, 6 bp, Artificial Sequence
  Synthesized
actccg

SEQ ID NO: 17 - DNA, 558 bp, Zea mays
gtactgtaatatttatattatatataattataaactataatatttcaaaactatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctcgtgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct

SEQ ID NO: 18 - DNA, 508 bp, Zea mays
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct

SEQ ID NO: 19 - DNA, 508 bp, Zea mays
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctcgtgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgct

SEQ ID NO: 20 - DNA, 24 bp, Artificial Sequence
  Synthesized
caagtttgtacaaaaaagcaggct

SEQ ID NO: 21 - DNA, 24 bp, Artificial Sequence
  Synthesized
acccagctttcttgtacaaagtgg

SEQ ID NO: 22 - DNA, 22 bp, Artificial Sequence
  Synthesized
acaactttgtataataaagttg

SEQ ID NO: 23 - DNA, 22 bp, Artificial Sequence
  Synthesized
acaactttgtatagaaaagttg

SEQ ID NO: 24 - DNA, 24 bp, Artificial Sequence
  Synthesized
caagttcgtacaaaaaagcaggct

SEQ ID NO: 25 - DNA, 23 bp, Artificial Sequence
  Synthesized
caagtttgtacaaaaaggactct

SEQ ID NO: 26 - DNA, 24 bp, Artificial Sequence
  Synthesized
caagtgcatacaaaaaggactgct

SEQ ID NO: 27 - DNA, 95 bp, Zea mays
tcaccaccggcgagccacatcgagaacacgatcgagcacacaagcacgaagactcgtttaggagaaaccacaaaccaccaagccgtgcaagcacc

SEQ ID NO: 28 - DNA, 133 bp, Artificial Sequence
  Synthesized
tcaccaccggcgagccacatcgagaacacgatcgagcacacaagcacgaagactcgtttaggagaaaccacaaaccaccaagccgtgcaagcaccaagcttggtcacccggtccgggcctagaaggccagctt

SEQ ID NO: 29 - DNA, 61 bp, Artificial Sequence
  Synthesized
tcgaaggagatagaaccaattctctaaggaaatacttaaccatggtcgactggatccaaca

SEQ ID NO: 30 - DNA, 25 bp, Artificial Sequence
  Synthesized
tcgaaggagatagaaccgatccacc

SEQ ID NO: 31 - DNA, 665 bp, Artificial Sequence
  Synthesized
ctatagtattttaaaattgcattaacaaacatgtcctaattggtactcctgagatactataccctcctgttttaaaatagttggcattatcgaattatcattttactttttaatgttttctcttcttttaatatattttatgaattttaatgtattttaaaatgttatgcagttcgctctggacttttctgctgcgcctacacttgggtgtactgggcctaaattcagcctgaccgaccgcctgcattgaataatggatgagcaccggtaaaatccgcgtacccaactttcgagaagaaccgagacgtggcgggccgggccaccgacgcacggcaccagcgactgcacacgtcccgccggcgtacgtgtacgtgctgttccctcactggccgcccaatccactcatgcatgcccacgtacacccctgccgtggcgcgcccagatcctaatcctttcgccgttctgcacttctgctgcctataaatggcggcatcgaccgtcacctgcttcaccaccggcgagccacatcgagaacacgatcgagcacacaagcacgaagactcgtttaggagaaaccacaaaccaccaagccgtgcaagcaccaagcttggtcacccggtccgggcctagaaggccagcttcaagtttgtacaaaaaagcaggct

SEQ ID NO: 32 - DNA, 19 bp, Artificial Sequence
  Synthesized
actctatcagtgatagagt

SEQ ID NO: 33 - DNA, 1272 bp, Artificial Sequence
  Synthesized
atgccccagttcgacatcctctgcaagaccccccccaaggtgctcgtgaggcagttcgtggagaggttcgagaggccctccggcgagaagatcgccctctgcgccgccgagctcacctacctctgctggatgatcacccacaacggcaccgccattaagagggccaccttcatgtcatacaacaccatcatctccaactccctctccttcgacatcgtgaacaagtccctccagttcaaatacaagacccagaaggccaccatcctcgaggcctccctcaagaagctcatccccgcctgggagttcaccatcatcccctactacggccagaagcaccagtccgacatcaccgacatcgtgtcatccctccagcttcagttcgagtcctccgaggaggctgacaagggcaactcccactccaagaagatgctgaaggccctcctctccgagggcgagtccatctgggagatcaccgagaagatcctcaactccttcgagtacacctccaggttcactaagaccaagaccctctaccagttcctcttcctcgccaccttcatcaactgcggcaggttctcagacatcaagaacgtggaccccaagtccttcaagctcgtgcagaacaagtacctcggcgtgatcatccagtgcctcgtgaccgagaccaagacctccgtgtccaggcacatctacttcttctccgctcgcggcaggatcgaccccctcgtgtacctcgacgagttcctcaggaactcagagcccgtgctcaagagggtgaacaggaccggcaactcctcctccaacaagcaggagtaccagctcctcaaggacaacctcgtgaggtcctacaacaaggccctcaagaagaacgccccctactccatcttcgccatcaagaacggccccaagtcccacatcggtaggcacctcatgacctccttcctctcaatgaagggcctcaccgagctcaccaacgtggtgggcaactggtccgacaagagggcctccgccgtggccaggaccacctacacccaccagatcaccgccatccccgaccactacttcgccctcgtgtcaaggtactacgcctacgaccccatctccaaggagatgatcgccctcaaggacgagactaaccccatcgaggagtggcagcacatcgagcagctcaagggctccgccgagggctccatcaggtaccccgcctggaacggcatcatctcccaggaggtgctcgactacctctcctcctacatcaacaggaggatctga

SEQ ID NO: 34 - protein, 423 aa, Artificial Sequence
  Synthesized
MPQFDILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 35 - DNA, 1032 bp, Artificial Sequence
  Synthesized
atgtccaacctgctcacggttcaccagaaccttccggctcttccagtggacgcgacgtccgatgaagtcaggaagaacctcatggacatgttccgcgacaggcaagcgttcagcgagcacacctggaagatgctgctctccgtctgccgctcctgggctgcatggtgcaagctgaacaacaggaagtggttccccgctgagcccgaggacgtgagggattaccttctgtacctgcaagctcgcgggctggcagtgaagaccatccagcaacaccttggacaactgaacatgcttcacaggcgctccggcctcccgcgccccagcgactcgaacgccgtgagcctcgtcatgcgccgcatcaggaaggaaaacgtcgatgccggcgaaagggcaaagcaggccctcgcgttcgagaggaccgatttcgaccaggtccgcagcctgatggagaacagcgacaggtgccaggacattaggaacctggcgttcctcggaattgcatacaacacgctcctcaggatcgcggaaattgcccgcattcgcgtgaaggacattagccgcaccgacggcggcaggatgcttatccacattggcaggaccaagacgctcgtttccaccgcaggcgtcgaaaaggccctcagcctcggagtgaccaagctcgtcgaacgctggatctccgtgtccggcgtcgcggacgacccaaacaactacctcttctgccgcgtccgcaagaacggggtggctgcccctagcgccaccagccaactcagcacgagggccttggaaggtattttcgaggccacccaccgcctgatctacggcgcgaaggatgacagcggtcaacgctacctcgcatggtccgggcactccgcccgcgttggagctgctagggacatggcccgcgccggtgtttccatccccgaaatcatgcaggcgggtggatggacgaacgtgaacattgtcatgaactacattcgcaaccttgacagcgagacgggcgcaatggttcgcctcctggaagatggtgactga

SEQ ID NO: 36 - protein, 343 aa, Artificial Sequence
  Synthesized
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 37 - DNA, 34 bp, Artificial Sequence
  Synthesized
gaagttcctatactttctagagaataggaacttc

SEQ ID NO: 38 - DNA, 34 bp, Artificial Sequence
  Synthesized
gaagttcctatactcttttgagaataggaacttc

SEQ ID NO: 39 - DNA, 34 bp, Artificial Sequence
  Synthesized
gaagttcctatactttttgaagaataggaacttc

SEQ ID NO: 40 - DNA, 34 bp, Artificial Sequence
  Synthesized
gaagttcctatacttattgaagaataggaacttc

SEQ ID NO: 41 - DNA, 30 bp, Artificial Sequence
  Synthesized
agttcctatactctatgtagaataggaact

SEQ ID NO: 42 - DNA, 34 bp, Artificial Sequence
  Synthesized
gaagttcctatactttctggagaataggaacttc

SEQ ID NO: 43 - protein, 146 aa, Artificial Sequence
  Synthesized
MIEVKPINAEDTYEIRHRILRPNQPLEACMYETDSLGGTFHLGGYYRGKLISIASFNQAEHPELEGQKQYQLRGMATLEGYREQKAGSTLIRHAEELLRKKGADLLWCNARTSASGYYKKLGFSEQGEVYDTPPVGPHILMYKKLT

SEQ ID NO: 44 - protein, 146 aa, Artificial Sequence
  Synthesized
MLEVKPINAEDTYELRHKILRPNQPLEVCMYETDLLRGAFHLGGFYRGKLISIASFHQAEHSELQGQKQYQLRGMATLEGYREQKAGSSLIKHAEEILRKRGADLLWCNARTSASGYYKKLGFSEQGEVFDTPPVGPHILMYKRIT

SEQ ID NO: 45 - protein, 146 aa, Artificial Sequence
  Synthesized
MLEVKPINAEDTYELRHRILRPNQPIEACMYESDLLRGAFHLGGYYRGKLISIASFHQAEHSELQGQKQYQLRGMATLEGYREQKAGSSLIKHAEEILRKRGADLLWCNARTSASGYYKKLGFSEQGEIFETPPVGPHILMYKRIT

SEQ ID NO: 46 - protein, 146 aa, Artificial Sequence
  Synthesized
MIEVKPINAEDTYDLRHRVLRPNQPIEACMFESDLTRSAFHLGGFYGGKLISVASFHQAEHTELQGKKQYQLRGVATLEGYREQKAGSSLVKHAEEILRKRGADMIWCNARTSASGYYRKLGFSEQGEVFDTPPVGPHILMYKRIT

SEQ ID NO: 47 - DNA, 442 bp, Artificial Sequence
  Synthesized
catgatagaggtgaaaccgattaacgcagaggatacctatgaactaaggcatagaatactcagaccaaaccagccgatagaagcgtgtatgtttgaaagcgatttacttcgtggtgcatttcacttaggcggcttttacaggggcaaactgatttccatagcttcattccaccaggccgagcactcggaactccaaggccagaaacagtaccagctccgaggtatggctaccttggaaggttatcgtgagcagaaagcgggatcaactctagttaaacacgctgaagaaatccttcgtaagaggggggcggacatgctttggtgtaatgcgaggacatccgcctcaggctactacaaaaagttaggcttcagcgagcagggagagatatttgacacgccgccagtaggacctcacatcctgatgtataaaaggatcacataa

SEQ ID NO: 48 - protein, 146 aa, Artificial Sequence
  Synthesized
MIEVKPINAEDTYELRHRILRPNQPIEACMFESDLLRGAFHLGGFYRGKLISIASFHQAEHSELQGQKQYQLRGMATLEGYREQKAGSTLVKHAEEILRKRGADMLWCNARTSASGYYKKLGFSEQGEIFDTPPVGPHILMYKRIT

SEQ ID NO: 49 - DNA, 441 bp, Artificial Sequence
  Synthesized
atgatagaggtgaaaccgattaacgcagaggatacctatgaactaaggcatagaatactcagaccaaaccagccgatagaagcgtgtatgtttgaaagcgatttacttcgtggtgcatttcacttaggcggctattacgggggcaaactgatttccatagcttcattccaccaggccgagcactcagaactccaaggccagaaacagtaccagctccgaggtatggctaccttggaaggttatcgtgagcagaaggcgggatcgagtctaattaaacacgctgaagaaattcttcgtaagaggggggcggacttgctttggtgtaatgcgcggacatccgcctcaggctactacaaaaagttaggcttcagcgagcagggagaggtattcgacacgccgccagtaggacctcacatcctgatgtataaaaggatcacataa

SEQ ID NO: 50 - protein, 146 aa, Artificial Sequence
  Synthesized
MIEVKPINAEDTYELRHRILRPNQPIEACMFESDLLRGAFHLGGYYGGKLISIASFHQAEHSELQGQKQYQLRGMATLEGYREQKAGSSLIKHAEEILRKRGADLLWCNARTSASGYYKKLGFSEQGEVFDTPPVGPHILMYKRIT

SEQ ID NO: 51 - DNA, 1968 bp, Glycine max
  misc_feature (1)...(1968): HRA sequence
atgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtac

SEQ ID NO: 52 - DNA, 1917 bp, Zea mays
  misc_feature (1)...(1917): HRA sequence
cagtacacagtcctgccatcaccatccaggatcatatccttgaaagccccaccactagggatcataggcaacacatgctcctggtgtgggacgattatatccaagaggtacggccctggagtctcgagcatcttctttatcgctgcgcggacttcgttcttctttgtcacacggaccgctggaatgttgaaccctttggcgatcgtcacgaaatctggatatatctcactttcattctctgggtttcccaagtatgtgtgcgctctgttggccttatagaacctgtcctccaactgcaccaccatccccaggtgctggttgtttagcacaaagaccttcactgggaggttctcaattcggatcatagctagctcctgaacgttcatgagaaagctaccatctccatcgatgtcaacaacagtgacacctgggtttgccacagaagcaccagcagcagccggcaaaccaaatcccatagccccaagaccagctgaagacaaccactgccttggccgcttgtaagtgtagtactgtgccgcccacatctggtgctgcccaacacctgtgccgatgatggcctcgcctttcgtcagctcatcaagaacctgaatagcatattgtggctggatctcctcattagatgttttatacccaagggggaattccctcttctgctgatccaactcatcgttccatgagccaaagtcaaagctcttctttgatgtgcttccttcaagaagagcattcatgccctgcaaagcaagcttaacatctgcacagatggacacatgtggctgcttgttcttgccaatctcagccggatcaatatcaacgtgcacaatcttagccctgcttgcaaaagcctcaatcttccctgtcacgcgatcatcaaaccgcacaccaagtgcaagcaacagatcggccttatccactgcataatttgcatacaccgtcccatgcatacctagcatgcgcagagacagtgggtcgtcgctggggaagttgccgaggcccataagagtagttgtgaccgggattccagtcagctccacaaagcgtcgcaactcctcaccagatgctgcgcagccaccgcccacataaagaacagggcgccgcgattcaccaacaagacgcagcacctgctcaagcaactcagtcgcagggggcttgggaaggcgcgcaatgtacccaggcagactcatgggcttgtcccagacaggcaccgccatctgctgctggatgtccttggggatgtcgacaagcaccggccctggtcgaccagaggaggcgaggaagaaagcctcctgcacgacgcgggggatgtcgtcgacgtcgaggaccaggtagttgtgcttggtgatggagcgggtgacctcgacgatgggcgtctcctggaaggcgtcggtgccaatcatgcgtcgcgccacctgtcccgtgatggcgaccatggggacggaatcgagcagcgcgtcggcgagcgcggagactaggttggtggcgccggggccggaggtggcgatgcagacgccgacgcggcccgaggagcgcgcgtagccggaggcggcaaaggcctccccttgctcgtggcggaagaggtggttggcgatgacgggggagcgggtgagtgcctggtggatctccatggacgcgccgccggggtaggcgaagacgtcgcggacgccgcagcgctcgagggactcgacgaggatgtcagcacccttgcggggctcggtggggccccacggccggagcggggtggccgggggagccatcggcatggcgggtgacgccgctgagcacctgatgggcgcggcgagggcgcggcgggtggccaggaggtgcgcccggcgcctcgccttgggcgcagcggtagtggcgccagtgagcgcggtagacgcggcggcggcggtggccatg

SEQ ID NO: 53 - DNA, 2139 bp, Arabidopsis
  misc_feature (1)...(2139): HRA sequence
aaatacgtacctacgcaccctgcgctaccatccctagagctgcagcttatttttacaacaattaccaacaacaacaaacaacaaacaacattacaattactatttacaattacagtcgacccgggatccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtcgctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaatatgattcttgagagccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaattggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgagtggtggcactttcaacgatgtcataacggaaggagatggccggattaaatac

SEQ ID NO: 54 - DNA, 552 bp, Artificial Sequence
  Synthesized
atgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctga

SEQ ID NO: 55 - DNA, 2130 bp, Zea mays
  CDS (1)...(2130)
atggccactgtgaacaactggctcgctttctccctctccccgcaggagctgccgccctcccagacgacggactccacgctcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaagtccaactgcaacttgatacccagcactagcagcacagtttgctacgcgagctcagctgctagcaccggctaccatcaccagctgtaccagcccaccagctccgcgctccacttcgcggactccgtcatggtggcctcctcggccggtgtccacgacggcggttccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaaggacaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgtctcaaggaggccgaggccgcagcgtccgcgcagcaccaccacgccggcgtggtgagctacgacgtcggccgcatcgcctcgcagctcggcgacggcggagccctagcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacttgaacctgggcgcggccggcgcgcacgactttttctcggcagggcagcaggccgccgccgcagctgcgatgcacggcctggctagcatcgacagtgcgtcgctcgagcacagcaccggctccaactccgtcgtctacaacggcggggtcggcgatagcaacggcgccagcgccgttggcagcggcggtggctacatgatgccgatgagcgctgccggagcaaccactacatcggcaatggtgagccacgagcagatgcatgcacgggcctacgacgaagccaagcaggctgctcagatggggtacgagagctacctggtgaacgcggagaacaatggtggcggaaggatgtctgcatgggggaccgtcgtctctgcagccgcggcggcagcagcaagcagcaacgacaacattgccgccgacgtcggccatggcggcgcgcagctcttcagtgtctggaacgacacttaa

SEQ ID NO: 56 - protein, 709 aa, Zea mays
MATVNNWLAFSLSPQELPPSQTTDSTLISAATADHVSGDVCFNIPQDWSMRGSELSALVAEPKLEDFLGGISFSEQHHKSNCNLIPSTSSTVCYASSAASTGYHHQLYQPTSSALHFADSVMVASSAGVHDGGSMLSAAAANGVAGAASANGGGIGLSMIKNWLRSQPAPMQPRAAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAATTGLYHPYAQQPMRGGGWCKQEQDHAVIAAAHSLQDLHHLNLGAAGAHDFFSAGQQAAAAAAMHGLASIDSASLEHSTGSNSVVYNGGVGDSNGASAVGSGGGYMMPMSAAGATTTSAMVSHEQMHARAYDEAKQAAQMGYESYLVNAENNGGGRMSAWGTVVSAAAAAAASSNDNIAADVGHGGAQLFSVWNDT

SEQ ID NO: 57 - DNA, 2260 bp, Zea mays
cttccctaacctttgcactgtccaaaatggcttcctgatcccctcacttcctcgaatcaatctaagaagaaactcaagccgcaaccattaggggcagattaattgctgcactttcagataatcaaccatggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 58 - DNA, 2133 bp, Zea mays
  CDS (1)...(2133)
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgattaagaactggctgcggagccaaccggcgcccatgcagccgagggtggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggagccgtcgtcgtcacggcgccgaaggaggatagcggtggcagcggtgttgccggcgctctagtagccgtgagcacggacacgggtggcagcggcggcgcgtcggctgacaacacggcaaggaagacggtggacacgttcgggcagcgcacgtcgatttaccgtggcgtgacaaggcatagatggactgggagatatgaggcacatctttgggataacagttgcagaagggaagggcaaactcgtaagggtcgtcaagtctatttaggtggctatgataaagaggagaaagctgctagggcttatgatcttgctgctctgaagtactggggtgccacaacaacaacaaattttccagtgagtaactacgaaaaggagctcgaggacatgaagcacatgacaaggcaggagtttgtagcgtctctgagaaggaagagcagtggtttctccagaggtgcatccatttacaggggagtgactaggcatcaccaacatggaagatggcaagcacggattggacgagttgcagggaacaaggatctttacttgggcaccttcagcacccaggaggaggcagcggaggcgtacgacatcgcggcgatcaagttccgcggcctcaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagctacgacgtcggccgcatcgcctcgcagctcggcgacggcggagccctggcggcggcgtacggcgcgcactaccacggcgccgcctggccgaccatcgcgttccagccgggcgccgccagcacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 59 - protein, 710 aa, Zea mays
MATVNNWLAFSLSPQELPPSQTTDSTLISAATADHVSGDVCFNIPQDWSMRGSELSALVAEPKLEDFLGGISFSEQHHKANCNMIPSTSSTVCYASSGASTGYHHQLYHQPTSSALHFADSVMVASSAGVHDGGAMLSAAAANGVAGAASANGGGIGLSMIKNWLRSQPAPMQPRVAAAEGAQGLSLSMNMAGTTQGAAGMPLLAGERARAPESVSTSAQGGAVVVTAPKEDSGGSGVAGALVAVSTDTGGSGGASADNTARKTVDTFGQRTSIYRGVTRHRWTGRYEAHLWDNSCRREGQTRKGRQVYLGGYDKEEKAARAYDLAALKYWGATTTTNFPVSNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHHAGVVSYDVGRIASQLGDGGALAAAYGAHYHGAAWPTIAFQPGAASTGLYHPYAQQPMRGGGWCKQEQDHAVIAAAHSLQDLHHLNLGAAGAHDFFSAGQQAAAAAMHGLGSIDSASLEHSTGSNSVVYNGGVGDSNGASAVGGSGGGYMMPMSAAGATTTSAMVSHEQVHARAYDEAKQAAQMGYESYLVNAENNGGGRMSAWGTVVSAAAAAAASSNDNMAADVGHGGAQLFSVWNDT

SEQ ID NO: 60 - DNA, 3727 bp, Zea mays
atggccactgtgaacaactggctcgctttctccctctccccgcaggagctgccgccctcccagacgacggactccacactcatctcggccgccaccgccgaccatgtctccggcgatgtctgcttcaacatcccccaagattggagcatgaggggatcagagctttcggcgctcgtcgcggagccgaagctggaggacttcctcggcggcatctccttctccgagcagcatcacaaggccaactgcaacatgatacccagcactagcagcacagtttgctacgcgagctcaggtgctagcaccggctaccatcaccagctgtaccaccagcccaccagctcagcgctccacttcgcggactccgtaatggtggcctcctcggccggtgtccacgacggcggtgccatgctcagcgcggccgccgctaacggtgtcgctggcgctgccagtgccaacggcggcggcatcgggctgtccatgatcaagaactggctgcggagccaaccggcgcccatgcagccgagggcggcggcggctgagggcgcgcaggggctctctttgtccatgaacatggcggggacgacccaaggcgctgctggcatgccacttctcgctggagagcgcgcacgggcgcccgagagtgtatcgacgtcagcacagggtggtgccgtcgtcgtcacggcgccgaaggaggatagcggtggcagcggtgttgccggtgctctagtagccgtgagcacggacacgggtggcagcggcggcgcgtcggctgacaacacggcaaggaagacggtggacacgttcgggcagcgcacgtcgatttaccgtggcgtgacaaggtaagggggtggatgaatcaagtaatcatgaaattttgaaaagccattggtaatccaaggaactgtcatgatagatttgattgcatctagacatagttccgatcgaatcaaatgagtaggccaatgtttagcctttggggatctcgctgattattaggagtaccattgtattgggcatggttgtggtatagtagtagacaattaacaaaaaagctaccacttttcaattattttaggcatagatggactgggagatatgaggcacatctttgggataacagttgcagaagggaaggacaaactcgtaagggtcgtcaaggtatacaaatataatgcaacatactgtcattaaatatgctttttctgtaagttttatatttcaccaatgatgttgttattgttaactgacattgcttcacactatcaattttggattcggcgcaatgatttgtgggattgaaatcaaatcttaaatctacagtctatttaggtacgcgatttctctccaactacttaatgcagttcgtttctccctataaccatattctttttcatctcaaatctcactcgactctttttttttatcttgtaccattgataggtggctatgataaagaggagaaagctgctagggcttatgatcttgctgctctgaagtactggggtcccacaacaacaacaaatttcccagtatgtatatgtagcatccagttttactttactgaagttcatatctcgttatgggctataaatatgtatcaaatgatgtccattagctagtgatctggagtgaaggttctatagtaaagtaaacgctgtgtgcggagtgcagtagcgggaggtctctcttctattttctaagaaaaatggacattgctgaaattgtacttaaagtcgtttattttatttttttgtatttccaggtgagtaactacgaaaaggagctcgaggacatgaagcacatgacaaggcaggagtttgtagcgtctctgagaaggtcggtctaacagcattgattaatcagtaccacctctactgaataaaatctgctgctatttgttaaattttgagcgaggtcaactgcatatttgatcttattagaccactgtatatgaatgcaggaagagcagtggtttctccagaggtgcatccatttacaggggagtgactaggtatgaattcatatagctaagaacttaacatcaacaaaaacacacatacacttgggttgatgtggcagatgcatgcatggattgaaaatgtgtgcatgttgttttacttgaactcgatctctgtatttataggcatcaccaacatggaagatggcaagcacggattggacgagttgcagggaacaaggatctttacttgggcaccttcagtaagtagcaaacaaatatgtttttgcattgtatatagagtacccttgaatatataaattcaccacatatacaagcaagttacagtcaactaacacaatctcaacgcaacgagaaagcaagtgttccagctgatagtacacatttgtagaccagccgcatatggttgttttgtatgcatgatgactattaaaaatgtgaccatcgcattaagtcatgcaaagttgcattgcagtagtacattgcttagtgcatgctcctcaagtggcttttttcaaacctgatcccatgtctggtgctattgttgtctcccattcacccgtgcatcaggtcaaaatagtaccatgcctgaataagaaaaacaaaacgagcatgcactggcagcagcagactaataaacaaagttccagcatttactaataaactaattaggctacagcatccaaaagattcttccaattaagccacaactgttcatgcatacatgggtatgccacccaggataccatgcatgcaccgtgcacgacgaaagcgaaacgctcgttctcggaatattagaactgacgaagccgagtgcaaccttctgtcgtggatgcaggcacccaggaggaggcagcggaggcgtacgacatcgcggcgatcaagttccgcggcctaaacgccgtcaccaacttcgacatgagccgctacgacgtgaagagcatcctggacagcagcgccctccccatcggcagcgccgccaagcgcctcaaggaggccgaggccgcagcgtccgcgcagcaccaccacgccggcgtggtgagttacgacgtcggccgcatcgcctcgcagctcggcgacggcggagccctggcggcggcgtacggcgcgcactaccacggcgccgcctggccgaccatcgcgttccagccgggcgccgccaccacaggcctgtaccacccgtacgcgcagcagccaatgcgcggcggcgggtggtgcaagcaggagcaggaccacgcggtgatcgcggccgcgcacagcctgcaggacctccaccacctgaacctgggcgcggccggcgcgcacgactttttctcggcagggcagcaggccgccgccgctgcgatgcacggcctgggtagcatcgacagtgcgtcgctcgagcacagcaccggctccaactccgtcgtctacaacggcggggtcggcgacagcaacggcgccagcgccgtcggcggcagtggcggtggctacatgatgccgatgagcgctgccggagcaaccactacatcggcaatggtgagccacgagcaggtgcatgcacgggcctacgacgaagccaagcaggctgctcagatggggtacgagagctacctggtgaacgcggagaacaatggtggcggaaggatgtctgcatgggggactgtcgtgtctgcagccgcggcggcagcagcaagcagcaacgacaacatggccgccgacgtcggccatggcggcgcgcagctcttcagtgtctggaacgacacttaa

SEQ ID NO: 61 - protein, 8 aa, Artificial Sequence
  Synthesized
PKXXXFLG

SEQ ID NO: 62 - protein, 13 aa, Artificial Sequence
  Synthesized
SSTLPXGGXAXXX

SEQ ID NO: 63 - protein, 9 aa, Artificial Sequence
  Synthesized
NWLXFSLSP

SEQ ID NO: 64 - protein, 63 aa, Artificial Sequence
  Synthesized
SXYRGVTRHHQHGRWQARIGRVAGNKDLYLGTFSTXEEAAEAYDXAAIKFRGLNAVTNFXXXR

SEQ ID NO: 65 - protein, 68 aa, Artificial Sequence
  Synthesized
SXYRGVTRHRWTGRYEAHLWDNSCRXEGQXRKXXXGGYDKEXKAARAYDLAALKYWGXXTXXNFPXSN

SEQ ID NO: 66 - protein, 31 aa, Artificial Sequence
  Synthesized
YEKELEEMKXMTRQEXXAXLRRKSSGFSRGA

SEQ ID NO: 67 - protein, 10 aa, Artificial Sequence
  Synthesized
XLSMIKXWLR

SEQ ID NO: 68 - protein, 7 aa, Artificial Sequence
  Synthesized
WCKXEQD

SEQ ID NO: 69 - protein, 8 aa, Artificial Sequence
  Synthesized
PXFXXWND

SEQ ID NO: 70 - protein, 5 aa, Artificial Sequence
  Synthesized
LXLSM

SEQ ID NO: 71 - protein, 7 aa, Artificial Sequence
  Synthesized
WPTIAFQ

SEQ ID NO: 72 - protein, 11 aa, Artificial Sequence
  Synthesized
SXGSNSVVYNG

SEQ ID NO: 73 - protein, 7 aa, Artificial Sequence
  Synthesized
QDWXMRG

SEQ ID NO: 74 - DNA, 1755 bp, Arabidopsis thaliana
  CDS (1)...(1755)
atgaactcgatgaataactggttaggcttctctctctctcctcatgatcaaaatcatcaccgtacggatgttgactcctccaccaccagaaccgccgtagatgttgccggagggtactgttttgatctggccgctccctccgatgaatcttctgccgttcaaacatcttttctttctcctttcggtgtcaccctcgaagctttcaccagagacaataatagtcactcccgagattgggacatcaatggtggtgcatgcaatacattaaccaataacgaacaaaatggaccaaagcttgagaatttcctcggccgcaccaccacgatttacaataccaacgagaccgttgtagatggaaatggcgattgtggaggaggagacggtggtggtggcggctcactaggcctttcgatgataaaaacatggctgagtaatcattcggttgctaatgctaatcatcaagacaatggtaacggtgcacgaggcttgtccctctctatgaattcatctactagtgatagcaacaactacaacaacaatgatgatgtcgtccaagagaagactattgttgatgtcgtagaaactacaccgaagaaaactattgagagttttggacaaaggacgtctatataccgcggtgttacaaggcatcggtggacaggtagatacgaggcacatttatgggacaatagttgcaaaagagaaggccagactcgcaaaggaagacaagtttatctgggaggttatgacaaagaagaaaaagcagctagggcttacgatttagccgcactaaagtattggggacccaccactactactaacttccccttgagtgaatatgagaaagaggtagaagagatgaagcacatgacgaggcaagagtatgttgcctctctgcgcaggaaaagtagtggtttctctcgtggtgcatcgatttatcgaggagtaacaaggcatcaccaacatggaaggtggcaagctaggatcggaagagtcgccggtaacaaagacctctacttgggaactttcggcacacaggaagaggctgctgaggcttatgacattgcagccattaaattcagaggattaagcgcagtgactaacttcgacatgaacagatacaatgttaaagcaatcctcgagagcccgagtctacctattggtagttctgcgaaacgtctcaaggacgttaacaatccggttccagctatgatgattagtaataacgtttcagagagtgcaaataatgttagcggttggcaaaacactgcgtttcagcatcatcagggaatggatttgagcttattgcagcaacagcaggagaggtacgttggttattacaatggaggaaacttgtctaccgagagtactagggtttgtttcaaacaagaggaggaacaacaacacttcttgagaaactcgccgagtcacatgactaatgttgatcatcatagctcgacctctgatgattctgttaccgtttgtggaaatgttgttagttatggtggttatcaaggattcgcaatccctgttggaacatcggttaattacgatccctttactgctgctgagattgcttacaacgcaagaaatcattattactatgctcagcatcagcaacaacagcagattcagcagtcgccgggaggagattttccggtggcgatttcgaataaccatagctctaacatgtactttcacggggaaggtggtggagaaggggctccaacgttttcagtttggaacgacacttag

SEQ ID NO: 75 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNTLTNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGPTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 76 - DNA, 1740 bp, Brassica napus
  CDS (1)...(1740)
atgaataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattag

SEQ ID NO: 77 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 78 - DNA, 1740 bp, Brassica napus
  CDS (1)...(1740)
atgaataataactggttaggcttttctctctctccttatgaacaaaatcaccatcgtaaggacgtctgctcttccaccaccacaaccgccgtagatgtcgccggagagtactgttacgatccgaccgctgcctccgatgagtcttcagccatccaaacatcgtttccttctccctttggtgtcgtcctcgatgctttcaccagagacaacaatagtcactcccgagattgggacatcaatggtagtgcatgtaataacatccacaatgatgagcaagatggaccaaaacttgagaatttccttggccgcaccaccacgatttacaacaccaacgaaaacgttggagatatcgatggaagtgggtgttatggaggaggagacggtggtggtggctcactaggactttcgatgataaagacatggctgagaaatcaacccgtggataatgttgataatcaagaaaatggcaatggtgcaaaaggcctgtccctctcaatgaactcatctacttcttgtgataacaacaactacagcagtaacaaccttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaacgagaaggccaaacgcgcaaaggaagacaagtttatttgggaggttatgacaaagaagaaaaagcagctagggcttatgatttagccgcactcaagtattggggaaccaccactactactaacttccccatgagcgaatatgagaaagagatagaagagatgaagcacatgacaaggcaagagtatgttgcctcacttcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaataatgctagcggttggcaaaacgctgcggttcagcatcatcagggagtagatttgagcttattgcagcaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcagatgattcggttactgtttgtggaaatgttgttggttatggtggttatcaaggatttgcagccccggttaactgcgatgcctacgctgctagtgagtttgactataacgcaagaaaccattattactttgctcagcagcagcagacccagcattcgccaggaggagattttcccgcggcaatgacgaataatgttggctctaatatgtattaccatggggaaggtggtggagaagttgctccaacatttacagtttggaacgacaattag

SEQ ID NO: 79 - protein, 579 aa, Brassica napus
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWNDN

SEQ ID NO: 80 - DNA, 2070 bp, Medicago truncatula
  CDS (1)...(2070)
atggcctctatgaacttgttaggtttctctctatctccacaagaacaacatccatcaacacaagatcaaacggtggcttcccgttttgggttcaaccctaatgaaatctcaggctctgatgttcaaggagatcactgctatgatctctcttctcacacaactcctcatcattcactcaacctttctcatcctttttccatttatgaagctttccacacaaataacaacattcacaccactcaagattggaaggagaactacaacaaccaaaacctactattgggaacatcatgcatgaaccaaaatgtgaacaacaacaaccaacaagcacaaccaaagctagaaaacttcctcggtggacactctttcaccgaccatcaagaatacggtggtagcaactcatactcttcattacacctcccacctcatcagccggaagcatcctgtggcggtggtgatggtagtacaagtaacaataactcaataggtttatctatgataaaaacatggctcagaaaccaaccaccaccaccagaaaacaacaacaataacaacaatgaaagtggtgcacgtgtgcagacactatcactttctatgagtactggctcacagtcaagttcatctgtgcctcttctcaatgcaaatgtgatgagtggtgagatttcctcatcggaaaacaaacaaccacccacaactgcagttgtacttgatagcaaccaaacaagtgtcgttgaaagtgctgtgcctagaaaatccgttgatacatttggacaaagaacttccatttaccgtggtgtaacaaggcatagatggacagggagatatgaagctcacctttgggataatagttgtagaagagaggggcagactcgcaaaggaaggcaagtttacttgggaggttatgacaaagaagaaaaagcagctagagcctatgatttggcagcactaaaatattggggaacaactactacaacaaattttccaattagccattatgaaaaagaagtggaagaaatgaagcatatgacaaggcaagagtacgttgcgtcattgagaaggaaaagtagtggtttttcacgaggtgcatccatttaccgaggagtaacaagacatcatcaacatggtagatggcaagctaggattggaagagttgcaggcaacaaagatctctacctaggaactttcagcactcaagaagaggcagcagaggcatatgatgtggcagcaataaaattcagaggactgagtgcagttacaaactttgacatgagcagatatgatgtcaaaaccatacttgagagcagcacattaccaattggtggtgctgcaaagcgtttaaaagacatggagcaagttgaattgaatcatgtgaatgttgatattagccatagaactgaacaagatcatagcatcatcaacaacacttcccatttaacagaacaagccatctatgcagcaacaaatgcatctaattggcatgcactttcattccaacatcaacaaccacatcatcattacaatgccaacaacatgcagttacagaattatccttatggaactcaaactcaaaagctttggtgcaaacaagaacaagattctgatgatcatagtacttatactactgctactgatattcatcaactacagttagggaataataataacaatactcacaatttctttggtttacaaaatatcatgagtatggattctgcttccatggataatagttctggatctaattctgttgtttatggtggtggagatcatggtggttatggaggaaatggtggatatatgattccaatggctattgcaaatgatggtaaccaaaatccaagaagcaacaacaattttggtgagagtgagattaaaggatttggttatgaaaatgtttttgggactactactgatccttatcatgcacaggcagcaaggaacttgtactatcagccacaacaattatctgttgatcaaggatcaaattgggttccaactgctattccaacacttgctccaaggactaccaatgtctctctatgtcctcctttcactttgttgcatgaatag

SEQ ID NO: 81 - protein, 689 aa, Medicago truncatula
MASMNLLGFSLSPQEQHPSTQDQTVASRFGFNPNEISGSDVQGDHCYDLSSHTTPHHSLNLSHPFSIYEAFHTNNNIHTTQDWKENYNNQNLLLGTSCMNQNVNNNNQQAQPKLENFLGGHSFTDHQEYGGSNSYSSLHLPPHQPEASCGGGDGSTSNNNSIGLSMIKTWLRNQPPPPENNNNNNNESGARVQTLSLSMSTGSQSSSSVPLLNANVMSGEISSSENKQPPTTAVVLDSNQTSVVESAVPRKSVDTFGQRTSIYRGVTRHRWTGRYEAHLWDNSCRREGQTRKGRQVYLGGYDKEEKAARAYDLAALKYWGTTTTTNFPISHYEKEVEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKTILESSTLPIGGAAKRLKDMEQVELNHVNVDISHRTEQDHSIINNTSHLTEQAIYAATNASNWHALSFQHQQPHHHYNANNMQLQNYPYGTQTQKLWCKQEQDSDDHSTYTTATDIHQLQLGNNNNNTHNFFGLQNIMSMDSASMDNSSGSNSVVYGGGDHGGYGGNGGYMIPMAIANDGNQNPRSNNNFGESEIKGFGYENVFGTTTDPYHAQAARNLYYQPQQLSVDQGSNWVPTAIPTLAPRTTNVSLCPPFTLLHE

SEQ ID NO: 82 - DNA, 2133 bp, Glycine max
  CDS (1)...(2133)
atggggtctatgaatttgttaggtttttctctctctcctcaagaacacccttctagtcaagatcactctcaaacggcaccttctcgtttttgcttcaaccctgatggaatctcaagcactgatgtagcaggagactgctttgatctcacttctgactcaactcctcatttactcaaccttccctcttacggcatatacgaagcttttcataggagcaacaatattcacaccactcaagattggaaggagaactacaacagccaaaacttgctattgggaacttcatgcagcaaccaaaacatgaaccacaaccatcagcaacaacaacaacaacagccaaagcttgaaaacttcctcggtggacactcatttggtgaacatgagcaaccctacggtggtaactcagcctctacagaatacatgttcccggctcagccggtattggccggtggcggcggcggtggtagcaatagcagcaacacaagcaacagtagctccatagggttatccatgataaagacatggttgaggaaccaaccaccacactcagaaaacaacaataacaacaacaatgaaagtggtggcaatagtagaagcagtgtgcagcagactctatcactttccatgagtactggttcacaatcaagcacatcactaccccttctcactgctagtgtggataatggagagagttcttctgataacaaacaaccacataccacggctgcacttgatacaacccaaaccggagccattgaaactgcacccagaaagtccattgacacttttggacagagaacttctatctaccgtggtgtaacaaggcataggtggacggggaggtatgaggctcacctgtgggataatagttgtagaagagagggacaaactcgcaaaggaaggcaagtttacttgggaggttatgacaaagaagaaaaggcagctagagcctacgatttggcagcactaaaatactggggaacaactacgacaacaaattttccaattagccactatgagaaagagttggaagaaatgaagcacatgactaggcaagagtacgttgcgtcattgagaaggaagagtagtgggttttctcgcggggcatccatttatcgaggtgtgacgagacaccatcaacatggaagatggcaagcgaggattggaagagttgctggcaacaaggatctctacttgggaactttcagcacccaagaggaggcagcagaagcatatgatgtagcagcaatcaaattcagaggactaagtgctgttacaaactttgacatgagcagatatgacgtgaaaagcatacttgagagcaccactttgccaattggtggtgctgcaaagcgtttgaaggatatggagcaggtggaactgagggtggagaatgttcatagagcagatcaagaagatcatagtagcatcatgaactctcacttaactcaaggaatcattaacaactatgcagcaggaggaacaacagcgactcatcatcataactggcacaatgctcttgcattccaccaacctcaaccttgcaccaccatacactacccttatggacaaagaattaattggtgcaagcaagaacaagacaactctgatgcctctcactctttgtcttattcagatattcatcaactacagctagggaacaatggcacacacaacttctttcacacaaattcagggttgcaccctatgttaagcatggattctgcttccattgacaatagctcttcatctaactctgttgtttatgatggttatggaggtggtgggggctataatgtgattcctatggggactactactactgttgttgcaaatgatggtgatcaaaatccaagaagcaatcatggttttggtgataatgagataaaggcacttggttatgaaagtgtgtatggttctacaactgatccttatcatgcacatgcaaggaacttgtattatcttactcaacagcaaccatcttctgttgatgcagtgaaggctagtgcatatgatcaaggatctgcatgcaatacttgggttccaactgctattccaactcatgcaccaaggtctagtactagtatggctctctgccatggtgctacgcccttctctttattgcatgaatag

SEQ ID NO: 83 - protein, 710 aa, Glycine max
MGSMNLLGFSLSPQEHPSSQDHSQTAPSRFCFNPDGISSTDVAGDCFDLTSDSTPHLLNLPSYGIYEAFHRSNNIHTTQDWKENYNSQNLLLGTSCSNQNMNHNHQQQQQQQPKLENFLGGHSFGEHEQPYGGNSASTEYMFPAQPVLAGGGGGGSNSSNTSNSSSIGLSMIKTWLRNQPPHSENNNNNNNESGGNSRSSVQQTLSLSMSTGSQSSTSLPLLTASVDNGESSSDNKQPHTTAALDTTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELRVENVHRADQEDHSSIMNSHLTQGIINNYAAGGTTATHHHNWHNALAFHQPQPCTTIHYPYGQRINWCKQEQDNSDASHSLSYSDIHQLQLGNNGTHNFFHTNSGLHPMLSMDSASIDNSSSSNSVVYDGYGGGGGYNVIPMGTTTTVVANDGDQNPRSNHGFGDNEIKALGYESVYGSTTDPYHAHARNLYYLTQQQPSSVDAVKASAYDQGSACNTWVPTAIPTHAPRSSTSMALCHGATPFSLLHE

SEQ ID NO: 84 - DNA, 1932 bp, Vitis vinifera
  CDS (1)...(1932)
atggcttccatgaacaactggttgggtttctctttgtcccctcgagaacttccaccacagcctgaaaatcactcacagaacagtgtctctagacttggtttcaactctgatgaaatctctgggactgatgtgtcaggtgagtgttttgatctcacttcagattccactgctccctctctcaacctccctcccccttttgggatacttgaagcattcaacaggaataatcagccccaagatactaactacaaaaccaccacttctgagctctccatgctcatgggtagttcatgcagtagtcatcataacctcgaaaaccaagaacccaaacttgaaaatttcctgggctgccgctcttttgctgatcatgagcagaaacttcaagggtactacatttccattggtttatccatgatcaagacatggctgcggaaccaacctgcacccacccatcaggataacaacaagagtactgatactgggcctgtcggtggagccgccgctgggaacctacccaatgcacagaccttatcgttgtccatgagcaccggctcgcaccagaccggtgccattgaaacggtgccaaggaagtccattgatacatttggacagaggacatccatataccgtggtgtaacaaggcatagatggacgggtagatatgaggctcatctatgggacaacagttgcagaagagaaggacaaactcgaaagggaaggcaagtttatttaggtggttatgacaaagaagaaaaggcagctagggcttacgatttagcagcactgaagtattggggtaccaccaccacaacaaatttccctattagcaactatgaaaaagagatagaggagatgaagcacatgacaaggcaggagtacgtagcatctctgcgaaggaagagtagcgggttttctcgtggagcatccatatatagaggagtgaccagacaccatcagcatgggagatggcaggcaaggattggaagagtcgcaggcaacaaagatctttacttgggaactttcagcacccaagaggaagcagcagaggcctatgacattgctgccattaagtttcgaggattgaatgcggtgaccaactttgatatgagtagatatgatgttaatagcattctagagagcagtaccttgccgattggtggagctgcaaagcggttgaaagatgctgagcaggctgaaatgactatagatggacagaggacagacgatgagatgagctcacagctgactgatggaatcaacaactatggagcacaccaccatggctggcctactgttgcattccaacaagctcagccatttagcatgcactacccttatggccatcagcagagggctgtttggtgtaagcaagagcaagaccctgatggcacacacaactttcaagatcttcaccaactacaattgggaaacactcacaacttcttccagcctaatgttctgcacaacctcatgagcatggactcttcttcaatggaccatagctcaggctccaattcagtcatctatagcggtggtggagccgctgatggcagcgctgcaactggcggcagtggcagtgggagcttccaaggggtaggttatgggaacaacattggctttgtgatgcccataagcaccgtcatcgctcatgaaggcggccatggccagggaaatggtggctttggagatagcgaagtgaaggcgattggttacgacaacatgtttggatcgacagatccttaccatgctaggagcttgtactatctttcacagcaatcatctgcaggcatggtgaagggcagtagtgcatatgatcaggggtcagggtgtaacaactgggttccaactgcagttccaaccctagctccaaggactaacagcttggcagtatgccatggaacacctacattcacagtatggaatgatacataa

SEQ ID NO: 85 - protein, 643 aa, Vitis vinifera
MASMNNWLGFSLSPRELPPQPENHSQNSVSRLGFNSDEISGTDVSGECFDLTSDSTAPSLNLPPPFGILEAFNRNNQPQDTNYKTTTSELSMLMGSSCSSHHNLENQEPKLENFLGCRSFADHEQKLQGYYISIGLSMIKTWLRNQPAPTHQDNNKSTDTGPVGGAAAGNLPNAQTLSLSMSTGSHQTGAIETVPRKSIDTFGQRTSIYRGVTRHRWTGRYEAHLWDNSCRREGQTRKGRQVYLGGYDKEEKAARAYDLAALKYWGTTTTTNFPISNYEKEIEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSILESSTLPIGGAAKRLKDAEQAEMTIDGQRTDDEMSSQLTDGINNYGAHHHGWPTVAFQQAQPFSMHYPYGHQQRAVWCKQEQDPDGTHNFQDLHQLQLGNTHNFFQPNVLHNLMSMDSSSMDHSSGSNSVIYSGGGAADGSAATGGSGSGSFQGVGYGNNIGFVMPISTVIAHEGGHGQGNGGFGDSEVKAIGYDNMFGSTDPYHARSLYYLSQQSSAGMVKGSSAYDQGSGCNNWVPTAVPTLAPRTNSLAVCHGTPTFTVWNDT

SEQ ID NO: 86 - DNA, 2088 bp, Oryza sativa
  CDS (1)...(2088)
atggccaccatgaacaactggctggccttctccctctccccgcaggatcagctcccgccgtctcagaccaactccactctcatctccgccgccgccaccaccaccaccgccggcgactcctccaccggcgacgtctgcttcaacatcccccaagattggagcatgaggggatcggagctctcggcgctcgtcgccgagccgaagctggaggacttcctcggcggcatctccttctcggagcagcagcatcatcacggcggcaagggcggcgtgatcccgagcagcgccgccgcttgctacgcgagctccggcagcagcgtcggctacctgtaccctcctccaagctcatcctcgctccagttcgccgactccgtcatggtggccacctcctcgcccgtcgtcgcccacgacggcgtcagcggcggcggcatggtgagcgccgccgccgccgcggcggccagtggcaacggcggcattggcctgtccatgatcaagaactggctccggagccagccggcgccgcagccggcgcaggcgctgtctctgtccatgaacatggcggggacgacgacggcgcagggcggcggcgccatggcgctcctcgccggcgcaggggagcgaggccggacgacgcccgcgtcagagagcctgtccacgtcggcgcacggagcgacgacggcgacgatggctggtggtcgcaaggagattaacgaggaaggcagcggcagcgccggcgccgtggttgccgtcggctcggagtcaggcggcagcggcgccgtggtggaggccggcgcggcggcggcggcggcgaggaagtccgtcgacacgttcggccagagaacatcgatctaccgcggcgtgacaaggcatagatggacagggaggtatgaggctcatctttgggacaacagctgcagaagagagggccaaactcgcaagggtcgtcaagtctatctaggtggttatgacaaagaggaaaaagctgctagagcttatgatttggctgctctcaaatactggggcccgacgacgacgacaaattttccggtaaataactatgaaaaggagctggaggagatgaagcacatgacaaggcaggagttcgtagcctctttgagaaggaagagcagtggtttctccagaggtgcatccatttaccgtggagtaactaggcatcaccagcatgggagatggcaagcaaggataggaagagttgcagggaacaaggacctctacttgggcaccttcagcacgcaggaggaggcggcggaggcgtacgacatcgcggcgatcaagttccgggggctcaacgccgtcaccaacttcgacatgagccgctacgacgtcaagagcatcctcgacagcgctgccctccccgtcggcaccgccgccaagcgcctcaaggacgccgaggccgccgccgcctacgacgtcggccgcatcgcctcgcacctcggcggcgacggcgcctacgccgcgcattacggccaccaccaccactcggccgccgccgcctggccgaccatcgcgttccaggcggcggcggcgccgccgccgcacgccgccgggctttaccacccgtacgcgcagccgctgcgtgggtggtgcaagcaggagcaggaccacgccgtgatcgcggcggcgcacagcctgcaggatctccaccacctcaacctcggcgccgccgccgccgcgcatgacttcttctcgcaggcgatgcagcagcagcacggcctcggcagcatcgacaacgcgtcgctcgagcacagcaccggctccaactccgtcgtctacaacggcgacaatggcggcggaggcggcggctacatcatggcgccgatgagcgccgtgtcggccacggccaccgcggtggcgagcagccacgatcacggcggcgacggcgggaagcaggtgcagatggggtacgacagctacctcgtcggcgcagacgcctacggcggcggcggcgccgggaggatgccatcctgggcgatgacgccggcgtcggcgccggccgccacgagcagcagcgacatgaccggagtctgccatggcgcacagctcttcagcgtctggaacgacacataa

SEQ ID NO: 87 - DNA, 2088 bp, Oryza sativa
  CDS (1)...(2088)
atggccactatgaacaactggctcgccttctcgctctcgccgcaggaccaactcccaccgtcgcagaccaatagcactctcatctccgctgctgcaaccaccacaaccgcaggcgattcgtcaacgggcgacgtctgcttcaacatccctcaagactggtccatgcgcggaagcgagcttagcgctctcgtcgcggagcccaagttggaggatttcttgggaggcatctccttctcggagcaacagcatcatcacggcggaaagggcggtgttatcccaagctctgctgccgcatgctatgcaagctccggctccagcgtgggctacctctaccctccgccttcatcctcgtcacttcagtttgcagacagcgtgatggtcgcaacctcatctccagtggttgcgcacgatggcgtgagcggtggcggtatggtctcagcagcagcggctgcagcagcttcgggtaatggcgggattggcctctccatgatcaagaactggctcaggagccaaccggctccgcaacctgcgcaagcactcagcctgtcgatgaacatggctggtactactaccgctcaaggtggaggcgcaatggcacttctcgcaggcgctggcgaaagaggaaggaccacaccagcatccgagagcctctctacttccgcgcacggagccaccacggctacaatggctggcgggaggaaagagatcaacgaggaaggatctggatccgctggtgccgtggttgcagttggctcagaatcaggtggatccggcgctgttgttgaagctggtgccgctgcggcagcggctcggaagagcgttgatactttcggccaaagaacgagcatctacagaggcgttactcggcaccgctggaccggcaggtacgaggcacacttgtgggacaacagctgtcgccgcgagggccaaactaggaagggaagacaggtctatctaggaggatatgacaaagaggagaaggctgccagagcgtacgacctggccgcgttgaagtactggggtccaacaacgacgaccaacttcccggtgaacaactacgagaaggagctggaagagatgaagcacatgacgcggcaggagttcgtcgcttctctcaggcgcaagtcatctggtttctccagaggtgcgtcgatctatagaggagttacccgccaccaccagcacggaaggtggcaggcaagaatcgggagagtcgccggtaacaaggacctgtacttgggaaccttctcgactcaggaggaggcagcggaagcgtatgacattgcggcgatcaagttccgcggtctcaatgccgtgaccaacttcgacatgtcacgctatgatgtcaagtcgattctggatagcgctgcgttgcctgtgggaaccgctgccaaacgcctcaaggacgcggaagcagctgccgcgtacgatgttggcaggattgcctcacatctcggtggagatggagcttacgctgcccactacgggcatcatcaccactctgcagccgcagcttggcctacaatagcattccaagcggcagcggctcctcctccacacgctgctggtctttaccatccgtacgcgcaacctctccgcggttggtgtaagcaggaacaagatcatgcggtgattgcggctgcacacagcttgcaagatctgcatcacctcaatctgggagccgcagcagctgcccatgacttcttctcacaagccatgcagcagcagcatggcctgggcagcatagacaatgcgtctctggagcactccaccggatcgaactcggtggtgtacaatggagacaacggcggaggaggtggaggttacatcatggcacctatgtcagcggtctctgctaccgctacggcggtggcctcatcccacgaccacggtggagacggcggcaagcaggtccaaatgggctacgactcctaccttgtgggagctgacgcttacggcggaggaggagctggtcgcatgcctagctgggccatgacgcctgcttctgctcctgcggctacgagctcgtcggatatgacaggagtgtgtcatggcgcccaactgttctcggtgtggaatgatacatag

SEQ ID NO: 88 - DNA, 4325 bp, Oryza sativa
atgcatatctatcttatataaatatctaccagtgatactgttgcttagtgctccaaacctctcttgacctcttcttcttcttctcagttagcttagcttaagcttcccctaaccttgagctcaccacaacaatggcgacttgatctaacagagcttaaccaagtagcaaatcatacatataaccatagcttaattcgcattgaatcttgtcttgttcagtgtgaatcatcaaccatggccaccatgaacaactggctggccttctccctctccccgcaggatcagctcccgccgtctcagaccaactccactctcatctccgccgccgccaccaccaccaccgccggcgactcctccaccggcgacgtctgcttcaacatcccccaaggtaattaagctcaccaatcgatgcatgcattcatgagctagatatagctagtgttggttgggatttgaagagacatgcatgtttgattgattgatttgatgtgcagattggagcatgaggggatcggagctctcggcgctcgtcgccgagccgaagctggaggacttcctcggcggcatctccttctcggagcagcagcatcatcacggcggcaagggcggcgtgatcccgagcagcgccgccgcttgctacgcgagctccggcagcagcgtcggctacctgtaccctcctccaagctcatcctcgctccagttcgccgactccgtcatggtggccacctcctcgcccgtcgtcgcccacgacggcgtcagcggcggcggcatggtgagcgccgccgccgccgcggcggccagtggcaacggcggcattggcctgtccatgatcaagaactggctccggagccagccggcgccgcagccggcgcaggcgctgtctctgtccatgaacatggcggggacgacgacggcgcagggcggcggcgccatggcgctcctcgccggcgcaggggagcgaggccggacgacgcccgcgtcagagagcctgtccacgtcggcgcacggagcgacgacggcgacgatggctggtggtcgcaaggagattaacgaggaaggcagcggcagcgccggcgccgtggttgccgtcggctcggagtcaggcggcagcggcgccgtggtggaggccggcgcggcggcggcggcggcgaggaagtccgtcgacacgttcggccagagaacatcgatctaccgcggcgtgacaaggtatttagggtgcaattaattaatcatctatctatattttgctcaaaaaagttcatctactagctagcttagcacaaatcatcatcagtgtaatcatatatattctttgatgatttaactgtgttgcatgaattcattcctatttgatgtttgtgatttggatcccattttctaggatagctatataggtgatagattgatcattagatttgtaggatttatcattatgtcattattatgtgggacatgattgttgtgattaacaaagttgtaatatcttttggtttggttataggcatagatggacagggaggtatgaggctcatctttgggacaacagctgcagaagagagggccaaactcgcaagggtcgtcaaggtaggctaactagtgccatttaaatcgattaattgtttttttatgctccaatggcgattgatactgatcttgtttctttttctaatgatcatttcgggatcgaatgatcttcctctgtttgatcgaacttggcttttgaatctacagtctatctaggtgagtgagattccttgaacctagatgttctgtttgcgatgcatgtatatattcggtagattgaattatttgctgatctttgctttcttgaagtttaatgatcttataaattgtaatgctgataggtggttatgacaaagaggaaaaagctgctagagcttatgatttggctgctctcaaatactggggcccgacgacgacgacaaattttccggtgtgtttataattaatatacagattgtgtcacattgttattttctcactcttttatttgatactgatctagtgtaatgatgattactaaaactgtacttaaaggcaatggtttctgtatttttcaggtaaataactatgaaaaggagctggaggagatgaagcacatgacaaggcaggagttcgtagcctctttgagaaggttggtctctacaatcaagatatccatactatactaattaatttccttttagatttatagtaatttatctatcgcattgaagttaattaattatctgatgcttactgatactaacaaatactgttccttatatgtgcaggaagagcagtggtttctccagaggtgcatccatttaccgtggagtaactaggtacatatatatatgcatcattgtacaattaatttttttaatttttttagggtaaaaaatgaagactgtgatatagatccattaatttgatcttgtgtacttgtaaatataggcatcaccagcatgggagatggcaagcaaggataggaagagttgcagggaacaaggacctctacttgggcaccttcagtaagtacaaatattcatatttatactgcaaaaccatataaatccatattaataagtatgtcctttctcattgagtatacaaaatatcatattttcttggcaagtacaatttattcattcagggcaaaatagtagtagtaagaaagaggggtgactcttcaaagaacacagagcttacttaagcctgtaactaattaattaaactaaaaatgtgatctgcaagtcatgtcaagttgcattacaccactaatatatatactctgtgcatgcttgcatgctctcctcatgtggctagctaccttttcaaaccttccatgtctggtgctactcctgtctccattcaccactgcacctggtcaagatcctcactaattaagaaacaataatgcattatttgcagtaaataatttaactagtgttaatcacattctttgcaacacaaactaatcaccaattaagctagctagctagccaaaatgataatcttgcttgcatgcgctaatggtgtgtgtgatgatggtggtgtcacgcatgcaggcacgcaggaggaggcggcggaggcgtacgacatcgcggcgatcaagttccgggggctcaacgccgtcaccaacttcgacatgagccgctacgacgtcaagagcatcctcgacagcgctgccctccccgtcggcaccgccgccaagcgcctcaaggacgccgaggccgccgccgcctacgacgtcggccgcatcgcctcgcacctcggcggcgacggcgcctacgccgcgcattacggccaccaccaccactcggccgccgccgcctggccgaccatcgcgttccaggcggcggcggcgccgccgccgcacgccgccgggctttaccacccgtacgcgcagccgctgcgtgggtggtgcaagcaggagcaggaccacgccgtgatcgcggcggcgcacagcctgcaggatctccaccacctcaacctcggcgccgccgccgccgcgcatgacttcttctcgcaggcgatgcagcagcagcacggcctcggcagcatcgacaacgcgtcgctcgagcacagcaccggctccaactccgtcgtctacaacggcgacaatggcggcggaggcggcggctacatcatggcgccgatgagcgccgtgtcggccacggccaccgcggtggcgagcagccacgatcacggcggcgacggcgggaagcaggtgcagatggggtacgacagctacctcgtcggcgcagacgcctacggcggcggcggcgccgggaggatgccatcctgggcgatgacgccggcgtcggcgccggccgccacgagcagcagcgacatgaccggagtctgccatggcgcacagctcttcagcgtctggaacgacacataaaaaaaaaactaggttagccagcttaattagcagggtaaaccactgacacaattaagccatacttaaattagggttcatgagatgaccattaagcaggttattatcattaatgatgtttaatttctcaattagtacttagctcaaaaggaggggatttcttctgaaggatggtgatggcttgtgaaattgaacctggtgttcttgccatgatttttttttcacaagctgccattttggggttcaggttcagaaggatcctgattattattaaccagccatatatatatagaagggtagaaatggaggtatcctgcttgtaaattggggcaatggtagctagagttgatgcaatgaccatgcttcatgtgatgagaactaattgtcttcctctgatcaaattaagcaggaagattaa

SEQ ID NO: 89 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVYLGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 90 - DNA, 1680 bp, Oryza sativa
  CDS (1)...(1680)
atggcctccatcaccaactggctcggcttctcctcctcctccttctccggcgccggcgccgaccccgtcctgccccacccgccgctgcaagagtgggggagcgcttatgagggcggcggcacggtggcggccgccggcggggaggagacggcggcgccgaagctggaggacttcctcggcatgcaggtgcagcaggagacggccgccgcggcggcggggcacggccgtggaggcagctcgtcggtcgttgggctgtccatgatcaagaactggctacgcagccagccgccgcccgcggtggttgggggagaagacgctatgatggcgctcgcggtgtcgacgtcggcgtcgccgccggtggacgcgacggtgccggcctgcatttcgccggatgggatggggtcgaaggcggccgacggcggcggcgcggccgaggcggcggcggcggcggcggcgcagaggatgaaggcggccatggacacgttcgggcagcggacgtccatctaccggggtgtcaccaagcacaggtggacaggaaggtatgaagcccatctttgggataacagctgcagaagagaaggtcagactcgcaaaggcagacaagtatatcttggaggatatgataaggaagaaaaagctgctagggcttatgatttggctgcccttaaatactggggcactacaacgacgacgaattttccggtaagcaactacgaaaaagagttggatgaaatgaagcacatgaataggcaggaatttgttgcatcccttagaagaaaaagcagtggattttcacgtggtgcttccatatatcgtggtgttacaagacaccatcagcatggaaggtggcaagcaaggataggacgggtggcaggaaacaaggatctgtatttgggcacatttggcacccaagaggaagctgcagaggcatatgatatcgctgcaatcaaattccgtggtctcaatgctgtgacaaactttgacatgagccggtacgatgtcaagagcatcattgaaagcagcaatctcccaattggtactggaaccacccggcgattgaaggactcctctgatcacactgataatgtcatggacatcaatgtcaataccgaacccaataatgtggtatcatcccacttcaccaatggggttggcaactatggttcgcagcattatggttacaatggatggtcgccaattagcatgcagccgatcccctcgcagtacgccaacggccagcccagggcatggttgaaacaagagcaggacagctctgtggttacagcggcgcagaacctgcacaatctacatcattttagttccttgggctacacccacaacttcttccagcaatctgatgttccagacgtcacaggtttcgttgatgcgccttcgaggtccagtgactcatactccttcaggtacaatggaacaaatggctttcatggtctcccgggtggaatcagctatgctatgccggttgcgacagcggtggaccaaggtcagggcatccatggctatggagaagatggtgtggcaggcattgacaccacacatgacctgtatggcagccgtaatgtgtactacctttccgagggttcgcttcttgccgatgtcgaaaaagaaggcgactatggccaatctgtggggggcaacagctgggttttgccgacaccgtag

SEQ ID NO: 91 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVYLGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 92 - DNA, 2112 bp, Oryza sativa
  CDS (1)...(2112)
atggcttctgcaaacaactggctgggcttctcgctctccggccaagagaatccgcagcctcaccaggatagctcgcctccggcagccatcgacgtctccggcgccggcgacttctatggcctgccgacgtcgcagccgacggcggccgacgcgcacctcggcgtggcggggcatcatcacaacgcctcgtatggcatcatggaggccttcaataggggagctcaagaggcacaagattggaacatgagggggctggactacaacggcggcgcctcggagctgtcgatgctcgtcggctccagcggcggcaagagggcggcggcggtggaggagaccgagccgaagctggaggacttcctcggcggcaactcgttcgtctccgagcaagatcatcacgcggcggggggcttcctcttctccggcgtcccgatggccagcagcaccaacagcaacagcgggagcaacactatggagctctccatgatcaagacctggctccggaacaacggccaggtgcccgccggccaccagccgcagcagcagcagccggcggccgcggccgccgccgcgcagcagcaggcgcacgaggcggcggagatgagcaccgacgcgagcgcgagcagcttcgggtgctcctccgacgcgatggggaggagtaacaacggcggcgcggtctcggcggcggccggcgggacgagctcgcagagcctggcgctctcgatgagcacgggctcgcactcgcacctgcctatcgtcgtcgccggcggcgggaacgccagcggcggagcggccgagagcacatcgtcggagaacaagcgggccagcggcgccatggattcgccgggcggtggcgcgatagaggccgtgccgaggaagtccatcgacacgttcgggcaaaggacctcgatatatcgaggtgtaacaaggcatagatggacagggcgatatgaggctcatctctgggataatagctgtagaagagaagggcagagtcgcaagggtaggcaagtttatcttggtggctatgacaaggaggataaagcagcgagagcttatgatttggcagctctgaagtattggggcacaacaacaacaacaaatttcccaataagtaactatgaaaaagagctagatgaaatgaaacatatgaccaggcaggagtatattgcatacctaagaaggaatagcagtggattttctcgtggtgcatcgaaatatcgtggtgtaaccaggcaccatcagcatgggagatggcaagcaaggatagggagggttgcaggaaacaaggacctctacttaggcaccttcagcaccgaggaggaggcggcggaggcgtacgacatcgcggcgatcaagttccgggggctcaacgccgtcaccaactttgacatgagccgctacgacgtcaagagcatcctggagagcagcacgctgccggtgggcggcgcggcgaggcggctgaaggaggcggcggaccacgcggaggcggccggcgccaccatctggcgcgccgccgacatggacggcgccggcgtcatctccggcctggccgacgtcgggatgggcgcctacgccgcctcgtaccaccaccaccaccaccacggctggccgaccatcgcgttccagcagccgccgccgctcgccgtgcactacccgtacggccaggcgccggcggcgccgtcgcgcgggtggtgcaagcccgagcaggacgccgccgtcgctgccgccgcgcacagcctccaggacctccagcagctgcacctcggcagcgccgccgcccacaacttcttccaggcgtcgtcgagctcgacggtctacaacggcggcggcggcgggtaccagggcctcggtggcaacgccttcttgatgccggcgagcaccgtcgtggccgaccaggggcacagcagcacggccaccaaccatggaaacacctgcagctacggcaacgaggagcaggggaagctcatcgggtacgacgccatggcgatggcgagcggcgccgccggcggcgggtaccagctgtcgcagggctcggcgtcgacggtgagcatcgcgagggcgaacggctactcggccaactggagctcgcctttcaatggcgccatgggatga

SEQ ID NO: 93 - protein, 703 aa, Oryza sativa
MASANNWLGFSLSGQENPQPHQDSSPPAAIDVSGAGDFYGLPTSQPTAADAHLGVAGHHHNASYGIMEAFNRGAQEAQDWNMRGLDYNGGASELSMLVGSSGGKRAAAVEETEPKLEDFLGGNSFVSEQDHHAAGGFLFSGVPMASSTNSNSGSNTMELSMIKTWLRNNGQVPAGHQPQQQQPAAAAAAAQQQAHEAAEMSTDASASSFGCSSDAMGRSNNGGAVSAAAGGTSSQSLALSMSTGSHSHLPIVVAGGGNASGGAAESTSSENKRASGAMDSPGGGAIEAVPRKSIDTFGQRTSIYRGVTRHRWTGRYEAHLWDNSCRREGQSRKGRQVYLGGYDKEDKAARAYDLAALKYWGTTTTTNFPISNYEKELDEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKEAADHAEAAGATIWRAADMDGAGVISGLADVGMGAYAASYHHHHHHGWPTIAFQQPPPLAVHYPYGQAPAAPSRGWCKPEQDAAVAAAAHSLQDLQQLHLGSAAAHNFFQASSSSTVYNGGGGGYQGLGGNAFLMPASTVVADQGHSSTATNHGNTCSYGNEEQGKLIGYDAMAMASGAAGGGYQLSQGSASTVSIARANGYSANWSSPFNGAMG

SEQ ID NO: 94 - DNA, 1977 bp, Oryza sativa
  CDS (1)...(1977)
atggcttctgcagataactggctaggcttctcgctctccggccaaggcaacccacagcatcaccagaacggctcgccgtctgccgccggcgacgccgccatcgacatctccggctcaggcgacttctatggtctgccaacgccggacgcacaccacatcggcatggcgggcgaagacgcgccctatggcgtcatggatgctttcaacagaggcacccatgaaacccaagattgggcgatgaggggtttggactacggcggcggctcctccgacctctcgatgctcgtcggctcgagcggcggcgggaggaggacggtggccggcgacggcgtcggcgaggcgccgaagctggagaacttcctcgacggcaactcattctccgacgtgcacggccaagccgccggcgggtacctctactccggaagcgctgtcggcggcgccggtggttacagtaacggcggatgcggcggcggaaccatagagctgtccatgatcaagacgtggctccggagcaaccagtcgcagcagcagccatcgccgccgcagcacgctgatcagggcatgagcaccgacgccagcgcgagcagctacgcgtgctccgacgtgctggtggggagctgcggcggcggcggcgccgggggcacggcgagctcgcatgggcagggcctggcgctgtcgatgagcacggggtcggtggccgccgccggagggggcggcgccgtcgtcgcggccgagagctcgtcgtcggagaacaagcgggtggattcgccgggcggcgccgtggacggcgccgtcccgaggaaatccatcgacaccttcgggcaaaggacgtctatataccgaggtgtaacaaggcatagatggacaggaagatatgaagctcatctgtgggataatagctgtaggagagaaggccaaagtcgcaaggggagacaggtttatttgggcggttatgacaaagaagataaggcggctcgggcttatgatttggcagctctaaaatactggggcacgaccacaacaacaaatttcccaatgagtaattatgaaaaggagctagaggaaatgaaacacatgaccaggcaggagtacattgcacatcttagaaggaatagcagtggattttctcgtggtgcatccaaatatcgtggtgttactaggcatcatcagcatgggagatggcaggcaaggatagggcgagttgcaggcaacaaggatatctacctaggcaccttcagcaccgaggaggaggccgccgaggcgtacgacatcgccgccatcaagttccgcgggctcaacgccgtcaccaacttcgacatgagccggtacgacgtcaagagcatcctggacagcagcacgctgccggtcggcggcgcggcgcggcggctcaaggaggcggaggtcgccgccgccgccgcgggcggcggcgtgatcgtctcccacctggccgacggcggtgtgggtgggtactactacgggtgcggcccgaccatcgcgttcggcggcggcggccagcagccggcgccgctcgccgtgcactacccgtcgtacggccaggccagcgggtggtgcaagccggagcaggacgcggtgatcgcggccgggcactgcgcgacggacctccagcacctgcacctcgggagcggcggcgccgccgccacccacaacttcttccagcagccggcgtcaagctcggccgtctacggcaacggcggcggcggcggcggcaacgcgttcatgatgccgatgggcgccgtggtggccgccgccgatcacggcgggcagagcagcgcctacggcggtggcgacgagagcgggaggctcgtcgtggggtacgacggcgtcgtcgacccgtacgcggccatgagaagcgcgtacgagctctcgcagggctcgtcgtcgtcgtcggtgagcgtcgcgaaggcggcgaacgggtacccggacaactggagctcgccgttcaacggcatgggatga

SEQ ID NO: 95 - protein, 658 aa, Oryza sativa
MASADNWLGFSLSGQGNPQHHQNGSPSAAGDAAIDISGSGDFYGLPTPDAHHIGMAGEDAPYGVMDAFNRGTHETQDWAMRGLDYGGGSSDLSMLVGSSGGGRRTVAGDGVGEAPKLENFLDGNSFSDVHGQAAGGYLYSGSAVGGAGGYSNGGCGGGTIELSMIKTWLRSNQSQQQPSPPQHADQGMSTDASASSYACSDVLVGSCGGGGAGGTASSHGQGLALSMSTGSVAAAGGGGAVVAAESSSSENKRVDSPGGAVDGAVPRKSIDTFGQRTSIYRGVTRHRWTGRYEAHLWDNSCRREGQSRKGRQVYLGGYDKEDKAARAYDLAALKYWGTTTTTNFPMSNYEKELEEMKHMTRQEYIAHLRRNSSGFSRGASKYRGVTRHHQHGRWQARIGRVAGNKDIYLGTFSTEEEAAEAYDIAAIKFRGLNAVTNFDMSRYDVKSILDSSTLPVGGAARRLKEAEVAAAAAGGGVIVSHLADGGVGGYYYGCGPTIAFGGGGQQPAPLAVHYPSYGQASGWCKPEQDAVIAAGHCATDLQHLHLGSGGAAATHNFFQQPASSSAVYGNGGGGGGNAFMMPMGAVVAAADHGGQSSAYGGGDESGRLVVGYDGVVDPYAAMRSAYELSQGSSSSSVSVAKAANGYPDNWSSPFNGMG

SEQ ID NO: 96 - DNA, 2112 bp, Sorghum bicolor
  CDS (1)...(2112)
atggctactgtgaacaactggctcgctttctccctctccccgcaggagctgccgcccacccagacggactccaccctcatctctgccgccaccaccgacgatgtctccggcgatgtctgcttcaacatcccccaagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggcatagatggactgggagatatgaagcacatctgtgggacaacagttgcagaagggaaggacaaactcgcaagggtcgtcaagtctatttaggtggctatgataaagaggagaaagctgctagggcttatgatctggctgctcttaagtactggggtcccacgacaacaacaaattttccagtgaataactacgaaaaggagctggaggatatgaagcacatgacaaggcaggagtttgtagcgtctctgagaaggaagagcagtggtttctccagaggtgcatccatttacaggggagtgactaggcatcaccagcatggaagatggcaagcacggattggacgagttgcagggaacaaggatctctacttgggcaccttcagcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatacttaa

SEQ ID NO: 97 - protein, 703 aa, Sorghum bicolor
MATVNNWLAFSLSPQELPPTQTDSTLISAATTDDVSGDVCFNIPQDWSMRGSELSALVAEPKLEDFLGGISFSEQHHKANCNMIPSTSSTACYASSGATAGYHHQLYHQPTSSALHFADSVMVASSAGGVHDGGAMLSAASANGSAGAGAASANGSGSIGLSMIKNWLRSQPAPMQPRVAAAESVQGLSLSMNMAGATQGAAGMPLLAGERGRAPESVSTSAQGGAVVTAPKEDSGGSGVAATGALVAVSTDTGGSGASADNTARKTVDTFGQRTSIYRGVTRHRWTGRYEAHLWDNSCRREGQTRKGRQVYLGGYDKEEKAARAYDLAALKYWGPTTTTNFPVNNYEKELEDMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSSALPIGSAAKRLKEAEAAASAQHHAGVVSYDVGRIASQLGDGGALAAAYGAHYHGAWPTIAFQPSAATGLYHPYAQPMRGWCKQEQDHAVIAAAHSLQELHHLNLGAAAGAHDFFSAGQQAAMHGLGSMDNASLEHSTGSNSVVYNGVGDSNGSTVVGSGGYMMPMSAATATATTAMVSHEQVHARAQGDHHDEAKQAAQMGYESYLVNAENYGGGRMSAAWATVSAPPAASSNDNMADVGHGGAQLFSVWNDT

SEQ ID NO: 98 - DNA, 3766 bp, Sorghum bicolor
atggctactgtgaacaactggctcgctttctccctctccccgcaggagctgccgcccacccagacggactccaccctcatctctgccgccaccaccgacgatgtctccggcgatgtctgcttcaacatcccccaaggtatgcatctatcgatcgatatatgtacgtacagtgcgcatatatatatatatctgcagtttgtggtacgaatactgattgaagctagcatgaaatgtcgtttgttctttcagattggagcatgaggggatccgagctttcggcgctcgtcgccgagccgaagctggaggacttcctcggcggaatctccttctccgagcagcaccacaaggccaactgcaacatgatccccagcactagcagcacagcttgctacgcgagctcgggtgctaccgccggctaccatcaccagctgtaccaccagcccaccagctccgcgctccacttcgctgactccgtcatggtggcctcctcggccggcggcgtccacgacggaggtgccatgctcagcgcggccagcgctaatggtagcgctggcgctggcgctgccagtgccaatggcagcggcagcatcgggctgtccatgatcaagaactggctgcggagccaaccagctcccatgcagccgagggtggcggcggctgagagcgtgcaggggctctctttgtccatgaacatggcgggggcgacgcaaggcgccgctggcatgccacttcttgctggagagcgcggccgggcgcccgagagtgtctcgacgtcggcacagggtggagccgtcgtcacggctccaaaggaggatagcggtggcagcggtgttgccgccaccggcgccctagtagccgtgagcacggacacgggtggcagcggcgcgtcggctgacaacacggcaaggaagacggtggacacgttcgggcagcgcacgtcgatttaccgtggcgtgacaaggtaataagggtccggtattacaatgaatcgtcacttcgtcagagaactaaactagcacaaatcagcaatgaatcaagtaatatcatgaaatttagaaaagccgttagcaatgcaaggagctatcattatagatttgattgcatctagacagttctgaattaaatgagtagggcaatgtgtagcctttgatgatctcgctgattattaggagtgccatttgtattggctatgattgtggtatatacagcagtagacaattaacaaaaggctaccactttcgaattattttaggcatagatggactgggagatatgaagcacatctgtgggacaacagttgcagaagggaaggacaaactcgcaagggtcgtcaaggtaccaatataatgcaatacaccgtatttaaatatatatgcttttctgtaattaagtttatactttcacaaaactgacattacttcgcattatcatttttggattgtcgtcgtcatgattggcgggattgaaatgaactattgaatctacagtctatttaggtaagcgatttcacttggttattaatttgggaccaactacttaatccagtttgtttttcccctataaccattattttttcatctgtgttctcaactcttacttttccatcttgttccactgataggtggctatgataaagaggagaaagctgctagggcttatgatctggctgctcttaagtactggggtcccacgacaacaacaaattttccagtatgtatatgtagaatgcagttttacttcactgaagatcatacctttgctatgtctcaaatgccgttcattagttagtggatctgaagtgaaggttctgtaatttttgttaactatgtacattgctggaattgtacttaaagtcatttgtttttgtatatctaggtgaataactacgaaaaggagctggaggatatgaagcacatgacaaggcaggagtttgtagcgtctctgagaaggtcggtcgaacagcattgattaatcaatgccaactctattgaataaacatctactctgttaattgttaaagtttgagagaaagatctgcatgttagatcttaatagaccactgtatatgaatgcaggaagagcagtggtttctccagaggtgcatccatttacaggggagtgactaggtatgaattcatataatggcgtcaacaaacacacatacactttgattgaggaggcgaatgcacgcatggattgaatgtgaatggtgttttacttgaactatgtaattataggcatcaccagcatggaagatggcaagcacggattggacgagttgcagggaacaaggatctctacttgggcaccttcagtaagtatcagagatgttttctcattgtatatagaggagtacttctatatgtatatatacattcagttattcaccacacaaaagcaaattgcagtcaactaataacaatctcaacgcaatgagaagcaagtgttacagctgatagtacacatttgtagaccttctgcatatggatgttatatatgatgactattaaaaatgtgaccattgcatcaagtcatgcaaagttgcattgcagtagtacatacattacttagtgcatgctcctcaagtggctttttcaaacctgatcccatgtctggcgctattgttgtctcccattcacccgtgcatcaggtcaaaatagtactatgcctcaataagaaacacatgagcatgcactggcagcagcagactaatcaagttctatcatttactaataaactaattaggctacagcatccaaaagattctacccattaagccacaactgttcatgcatgcattcataaaccaggataccaccatgcatgcgtgcaccgtgttcgtgcttggaatattgagctgagccgagtgcacccttgcgtggatgcaggcacgcaggaggaggcagcggaggcatacgacattgcggcgatcaagttccgcggcctcaacgccgtcacaaacttcgacatgagccgctacgacgtcaagagcatcctggacagcagtgcgctccccatcggcagcgccgccaagcgtctcaaggaggccgaggccgccgcgtccgcacagcaccatgccggcgtggtgagctacgacgtcggccgcatagcctcacagctcggcgacggcggcgccctggcggcggcgtacggcgcgcactaccatggcgcctggccgaccatcgcgttccagccgagcgcggccacgggcctgtaccacccgtacgcgcagccgatgcgcgggtggtgcaagcaggagcaggaccacgcggtgatcgcggccgcgcacagcctgcaggagctccaccacctgaacctgggtgctgccgccggcgcgcacgacttcttctcggcggggcagcaggcggcgatgcacggcctgggtagcatggacaatgcatcactcgagcacagcaccggctccaactccgtcgtgtacaacggtgttggtgatagcaacggcagcaccgtcgtcggcagtggtggctacatgatgcctatgagcgctgccacggcgacggctaccacggcaatggtgagccacgagcaggtgcatgcacgggcacagggtgatcaccacgacgaagccaagcaggctgctcagatggggtacgagagctacctggtgaacgcagagaactatggcggcgggaggatgtctgcggcctgggcgactgtctcagcgccaccggcggcaagcagcaacgataacatggcggacgtcggccatggcggcgcacagctcttcagtgtctggaacgatact

SEQ ID NO: 99 - DNA, 2082 bp, Sorghum bicolor
  CDS (1)...(2082)
atggcttcgacgaacaaccactggctgggtttctcgctctcgggccaggataacccgcagcctaatcatcaggacagctcgcctgccgccgccggcatcgacatctccggcgccagcgacttctatggcttgcccacgcagcagggctccgacgggaatctcggcgtgccgggcctgcgggacgatcacgcttcttatggcatcatggaggccttcaacagggttcctcaagaaacccaagattggaacatgaggggattggactacaacggcggtggctcggaactctcgatgcttgtggggtccagcggcggcggcgggggcggcggcaagagggccgtggaagacagcgagcccaagctcgaagatttcctcggcggcaactcgttcgtctccgagcatgatcagtccggcggttacctgttctctggagtcccgatggccagcagcaccaacagcaacagcgggagcaacaccatggagctctccatgatcaagacctggctccggaacaaccaggtgccccagccgcagccgccagcagctccgcatcaggcgccgcagactgaggagatgagcaccgacgccaacgccagcgccagcagctttggctgctcggattcgatggggaggaacggcacggtggcggctgctgggagctcccagagcctggcgctctcgatgagcacgggctcgcacctgccgatggttgtggccggcggcggcgccagcggagcggcctcggagagcacgtcatcggagaacaagcgagcgagcggcgccatggattcgcccggcagcgcggtagaagccgtcccgaggaagtccatcgacacgttcgggcaaaggacctctatatatcgaggtgtaacaagacatagatggacagggcgatatgaggctcatctatgggataatagttgtagaagagaagggcagagtcgcaagggtaggcaagtttaccttggtggctatgacaaggaagacaaggcagcaagggcttatgatttggcagctctcaagtattggggcactactacaacaacaaatttccctataagcaactatgaaaaggagctagaggaaatgaaacatatgactaggcaggagtatattgcatacctaagaagaaatagcagtggattttctcgtggcgcatcaaaatatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctgaacgccgtcaccaacttcgacatgagccgctacgacgtcaagagcatcctcgagagcagcacgctgcctgtcggcggcgcggccaggcgcctcaaggatgccgtggaccacgtggaggccggcgccaccatctggcgcgccgacatggacggcggcgtgatctcccagctcgccgaagccgggatgggcggctacgcctcgtacgggcaccacgcctggccgaccatcgcgttccagcagccgtcgccgctctccgtccactacccgtacgggcagccgccgtcccgcgggtggtgcaagcccgagcaggacgcggccgtcgccgccgccgcgcacagcctgcaggacctccagcagctgcacctcggcagcgcggcacacaacttcttccaggcgtcgtcgagctcggcagtctacaacagcggcggcggcggcgctagcggcgggtaccaccagggcctcggtggcggcagcagctccttcctcatgccgtcgagcactgtcgtggcgggggccgaccaggggcacagcagcagcacggccaaccaggggagcacgtgcagctacggggacgatcaccaggaagggaagctcatcgggtacgacgccatggtggcggcgaccgcagccggcggggacccgtacgccgcggcgaggagcgggtaccagttctcgtcgcagggctcgggatccacggtgagcatcgcgagggcgaacgggtactctaacaactggagctctcctttcaacggcggcatggggtga

SEQ ID NO: 100 - protein, 693 aa, Sorghum bicolor
MASTNNHWLGFSLSGQDNPQPNHQDSSPAAAGIDISGASDFYGLPTQQGSDGNLGVPGLRDDHASYGIMEAFNRVPQETQDWNMRGLDYNGGGSELSMLVGSSGGGGGGGKRAVEDSEPKLEDFLGGNSFVSEHDQSGGYLFSGVPMASSTNSNSGSNTMELSMIKTWLRNNQVPQPQPPAAPHQAPQTEEMSTDANASASSFGCSDSMGRNGTVAAAGSSQSLALSMSTGSHLPMVVAGGGASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGGVISQLAEAGMGGYASYGHHAWPTIAFQQPSPLSVHYPYGQPPSRGWCKPEQDAAVAAAAHSLQDLQQLHLGSAAHNFFQASSSSAVYNSGGGGASGGYHQGLGGGSSSFLMPSSTVVAGADQGHSSSTANQGSTCSYGDDHQEGKLIGYDAMVAATAAGGDPYAAARSGYQFSSQGSGSTVSIARANGYSNNWSSPFNGGMG

SEQ ID NO: 101 - DNA, 2040 bp, Zea mays
  CDS (1)...(2040)
atggcttcagcgaacaactggctgggcttctcgctctcgggccaggataacccgcagcctaaccaggatagctcgcctgccgccggtatcgacatctccggcgccagcgacttctatggcctgcccacgcagcagggctccgacgggcatctcggcgtgccgggcctgcgggacgatcacgcttcttatggtatcatggaggcctacaacagggttcctcaagaaacccaagattggaacatgaggggcttggactacaacggcggtggctcggagctctcgatgcttgtggggtccagcggcggcggcgggggcaacggcaagagggccgtggaagacagcgagcccaagctcgaagatttcctcggcggcaactcgttcgtctccgatcaagatcagtccggcggttacctgttctctggagtcccgatagccagcagcgccaatagcaacagcgggagcaacaccatggagctctccatgatcaagacctggctacggaacaaccaggtggcccagccccagccgccagctccacatcagccgcagcctgaggaaatgagcaccgacgccagcggcagcagctttggatgctcggattcgatgggaaggaacagcatggtggcggctggtgggagctcgcagagcctggcgctctcgatgagcacgggctcgcacctgcccatggttgtgcccagcggcgccgccagcggagcggcctcggagagcacatcgtcggagaacaagcgagcgagcggtgccatggattcgcccggcagcgcggtagaagccgtaccgaggaagtccatcgacacgttcgggcaaaggacctctatatatcgaggtgtaacaaggcatagatggacagggcggtatgaggctcatctatgggataatagttgtagaagggaagggcagagtcgcaagggtaggcaagtttaccttggtggctatgacaaggaggacaaggcagcaagggcttatgatttggcagctctcaagtattggggcactacgacaacaacaaatttccctataagcaactacgaaaaggagctagaagaaatgaaacatatgactagacaggagtacattgcatacctaagaagaaatagcagtggattttctcgtggggcgtcaaagtatcgtggagtaactagacatcatcagcatgggagatggcaagcaaggatagggagagttgcaggaaacaaggatctctacttgggcacattcagcaccgaggaggaggcggcggaggcctacgacatcgccgcgatcaagttccgcggtctcaacgccgtcaccaacttcgacatgagccgctacgacgtgaagagcatcctcgagagcagcacactgcctgtcggcggtgcggccaggcgcctcaaggacgccgtggaccacgtggaggccggcgccaccatctggcgcgccgacatggacggcgccgtgatctcccagctggccgaagccgggatgggcggctacgcctcgtacggccaccacggctggccgaccatcgcgttccagcagccgtcgccgctctccgtccactacccgtacggccagccgtcccgcgggtggtgcaaacccgagcaggacgcggccgccgccgcggcgcacagcctgcaggacctccagcagctgcacctcggcagcgcggcccacaacttcttccaggcgtcgtcgagctccacagtctacaacggcggcgccggcgccagtggtgggtaccagggcctcggtggtggcagctctttcctcatgccgtcgagcactgtcgtggcggcggccgaccaggggcacagcagcacggccaaccaggggagcacgtgcagctacggggacgaccaccaggaggggaagctcatcggttacgacgccgccatggtggcgaccgcagctggtggagacccgtacgctgcggcgaggaacgggtaccagttctcgcagggctcgggatccacggtgagcatcgcgagggcgaacgggtacgctaacaactggagctctcctttcaacaacggcatggggtga

SEQ ID NO: 102 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 103 - DNA, 975 bp, Zea mays
  CDS (1)...(975)
atggagacgccacagcagcaatccgccgccgccgccgccgccgccgcccacgggcaggacgacggcgggtcgccgccgatgtcgccggcctccgccgcggcggcggcgctggcgaacgcgcggtggaacccgaccaaggagcaggtggccgtgctggaggggctgtacgagcacggcctgcgcacccccagcgcggagcagatacagcagatcacgggcaggctgcgggagcacggcgccatcgagggcaagaacgtcttctactggttccagaaccacaaggcccgccagcgccagaggcagaagcaggacagcttcgcctacttcagcaggctcctccgccggcccccgccgctgcccgtgctctccatgccccccgcgccaccgtaccatcacgcccgcgtcccggcgccgcccgcgataccgatgccgatggcgccgccgccgcccgctgcatgcaacgacaacggcggcgcgcgtgtgatctacaggaacccattctacgtggctgcgccgcaggcgccccctgcaaatgccgcctactactacccacagccacagcagcagcagcagcagcaggtgacagtcatgtaccagtacccgagaatggaggtagccggccaggacaagatgatgaccagggccgcggcgcaccagcagcagcagcacaacggcgccgggcaacaaccgggacgcgccggccaccccagccgcgagacgctccagctgttcccgctccagcccaccttcgtgctgcggcacgacaaggggcgcgccgccaacggcagtaataacgactccctgacgtcgacgtcgacggcgactgcgacagcgacagcgacagcgacagcgtccgcttccatctccgaggactcggatggcctggagagcggcagctccggcaagggcgtcgaggaggcgcccgcgctgccgttctatgacttcttcgggctccagtcctccggaggccgctga

SEQ ID NO: 104 - protein, 324 aa, Zea mays
METPQQQSAAAAAAAAHGQDDGGSPPMSPASAAAAALANARWNPTKEQVAVLEGLYEHGLRTPSAEQIQQITGRLREHGAIEGKNVFYWFQNHKARQRQRQKQDSFAYFSRLLRRPPPLPVLSMPPAPPYHHARVPAPPAIPMPMAPPPPAACNDNGGARVIYRNPFYVAAPQAPPANAAYYYPQPQQQQQQQVTVMYQYPRMEVAGQDKMMTRAAAHQQQQHNGAGQQPGRAGHPSRETLQLFPLQPTFVLRHDKGRAANGSNNDSLTSTSTATATATATATASASISEDSDGLESGSSGKGVEEAPALPFYDFFGLQSSGGR

SEQ ID NO: 105 - DNA, 909 bp, Zea mays
  CDS (1)...(909)
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctgcaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 106 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 107 - DNA, 978 bp, Zea mays
  CDS (1)...(978)
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccttcaggcgcggcgcctccctcgcccaccctcggcttctacgccgccggcaatggcggcggatcggctgggctgctggacacgagttccgactggggcagcagcggcgctgctatggccaccgagacatgcttcctgcaggactacatgggcgtgacggacacgggcagctcgtcgcagtggccatgcttctcgtcgtcggacacgataatggcggcggcggcggccgcggcgcgggtggcgacgacgcgggcgcccgagacactccctctcttcccgacctgcggcgacgacgacgacgacgacagccagcccccgccgcggccgcggcacgcagtcccagtcccggcaggcgagaccatccgcggcggcggcggcagcagcagcagctacttgccgttctggggtgccggtgccgcgtccacaactgccggcgccacttcttccgttgcgatccagcagcaacaccagctgcaggagcagtacagcttttacagcaacagcacccagctggccggcaccggcagccaagacgtatcggcttcagcggccgccctggagctgagcctcagctcatggtgctccccttaccctgctgcagggagcatgtga

SEQ ID NO: 108 - protein, 325 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPSGAAPPSPTLGFYAAGNGGGSAGLLDTSSDWGSSGAAMATETCFLQDYMGVTDTGSSSQWPCFSSSDTIMAAAAAAARVATTRAPETLPLFPTCGDDDDDDSQPPPRPRHAVPVPAGETIRGGGGSSSSYLPFWGAGAASTTAGATSSVAIQQQHQLQEQYSFYSNSTQLAGTGSQDVSASAAALELSLSSWCSPYPAAGSM

SEQ ID NO: 109 - DNA, 663 bp, Zea mays
  CDS (1)...(663)
atggaggcgctgagcgggcgggtaggcgtcaagtgcgggcggtggaaccctacggcggagcaggtgaaggtcctgacggagctcttccgcgcggggctgcggacgcccagcacggagcagatccagcgcatctccacccacctcagcgccttcggcaaggtggagagcaagaacgtcttctactggttccagaaccacaaggcccgcgagcgccaccaccacaagaagcgccgccgcggcgcgtcgtcgtcctcccccgacagcggcagcggcaggggaagcaacaacgaggaagacggccgtggtgccgcctcgcagtcgcacgacgccgacgccgacgccgacctcgtgctgcaaccgccagagagcaagcgggaggccagaagctatggccaccatcaccggctcgtgacatgctacgtcagggacgtggtggagcagcaggaggcgtcgccgtcgtgggagcggccgacgagggaggtggagacgctagagctcttccccctcaagtcgtacggcgacctcgaggcggcggagaaggtccggtcgtacgtcagaggcatcgccgccaccagcgagcagtgcagggagttgtccttcttcgacgtctccgccggccgggatccgccgctcgagctcaggctctgcagcttcggtccctag

SEQ ID NO: 110 - protein, 220 aa, Zea mays
MEALSGRVGVKCGRWNPTAEQVKVLTELFRAGLRTPSTEQIQRISTHLSAFGKVESKNVFYWFQNHKARERHHHKKRRRGASSSSPDSGSGRGSNNEEDGRGAASQSHDADADADLVLQPPESKREARSYGHHHRLVTCYVRDVVEQQEASPSWERPTREVETLELFPLKSYGDLEAAEKVRSYVRGIAATSEQCRELSFFDVSAGRDPPLELRLCSFGP

SEQ ID NO: 111 - DNA, 896 bp, Zea mays
gtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctt

SEQ ID NO: 112 - DNA, 82 bp, Zea mays
tccccaacctcgtgttgttcggagcgcacacacacacaaccagatctcccccaaatccacccgtcggcacctccgcttcaag

SEQ ID NO: 113 - DNA, 1013 bp, Zea mays
gtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 114 - DNA, 11 bp, Triticum monococcum
cctcgttttgg

SEQ ID NO: 115 - DNA, 1036 bp, Triticum monococcum
atttgcctgatgagacgcttgacaacagtgtattgatggatgtctggtcggtatacacgcacagcacagtacccctactcctaggactggcgagtatctttcattcattccagaaatacgcgggtcggccaaaagtagaaaaatacactgcgcccactcaatccacgtagcgcactgcactgcacagcaacgcttcatgtcaaaagtcgagctcaagcatgcacgcgatggacgcggcgcgaatgacccgggcggcacgacgcgagtgcccgccgcgcccgcccgcctgccccgcagccgacctctcccaaacgggacaagcgagacggcccaaaacgagcaaggaaagcagcctcctactgtggcagcccgcccccacgaccgtcatctcaccttccattccattttccctggacggaccagacccgtccgagccgccctgacctagccagccagcatttcctctttcgtcccccgccgccgtgaccaaaaaagcaaaaaaggaaaaagggaaaatgctaaaggaaaaaactccgctctttcccttcttctaggcctagggtacagtagaatattataaaaggaaaaattctgctcgttttttgctctgtggtgtgtgtttgtggcgagagaaaatgatttggggaaagcaaaatcgggagattcgcacgtacgatcgttcgacacgtcgacgcccggcgggcccgtggtggggcatcgtgtggctgcaggaccgcggggccccgcggggcgggccgggccaatgggtgctcgacagcggctatgctccagaccagcccggtattgcataccgcgctcggggccagatccctttaaaaacccctccccccctgccggaaccctcgttttggcctggccatcctccctctcctcccctctcttccacctcacccaaccacctgatagccatggctccgccgcctcgcctccgcctgcgccagtcggagtagccgtcgcggtctgcgggtgttggagggtaggggcgtagggttggcccggttctcgagcggagatg

(19) United States
(12) Patent Application Publication
(10) Pub. No.: US 2014/0173781 A1
(43) Pub. Date: Jun. 19, 2014

(54) METHODS AND COMPOSITIONS FOR PRODUCING AND SELECTING TRANSGENIC WHEAT PLANTS

(71) Applicant: PIONEER HI-BRED INTERNATIONAL, INC., Johnston, IA (US)

(72) Inventors: Myeong-Je Cho, Alameda, CA (US); Samuel R. Ellis, Des Moines, IA (US); William James Gordon-Kamm, Urbandale, IA (US); Zuo-Yu Zhao, Johnston, IA (US)

(73) Assignee: PIONEER HI-BRED INTERNATIONAL, INC., Johnston, IA (US)

(21) Appl. No.: 13/801,418

(22) Filed: Mar. 13, 2013

Related U.S. Application Data
(60) Provisional application No. 61/736,947, filed on Dec. 13, 2012.

(51) Int. Cl. C12N 15/82

(52) U.S. Cl. CPC C12N 15/8237
USPC 800/300; 435/418; 435/468

(57) ABSTRACT

Compositions and methods are provided for the production and selection of transgenic plants and plant parts, for increasing the transformation frequency of a plant or plant part, and for regulating the expression of a transgene, such as a herbicide tolerance polynucleotide. The methods and compositions allow for the delay in the expression of herbicide tolerance polynucleotides until a point in development during which herbicide selection is more efficient. Compositions comprise polynucleotide constructs comprising an excision cassette that separates a transgene, such as a herbicide tolerance polynucleotide, from its promoter and host cells comprising the same. The excision cassette comprises a polynucleotide encoding a site-specific recombinase operably linked to an inducible promoter and expression of the recombinase leads to excision of the excision cassette and expression of the transgene.

### CROSS-REFERENCE TO RELATED APPLICATIONS

This application claims priority to U.S. Provisional Application No. 61/736,947, filed on Dec. 13, 2012, which is hereby incorporated by reference in its entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 430618seqlist.TXT, created on Mar. 12, 2013, and having a size of 308 kilobytes and is filed concurrently with the specification. The sequence listing contained in this ASCII formatted document is part of the specification and is herein incorporated by reference in its entirety.

### FIELD OF THE INVENTION

The present invention relates to the genetic modification of plants. More particularly, the compositions and methods are directed to the production and selection of transgenic plants.

### BACKGROUND OF THE INVENTION

Current genetic engineering technology allows for the production of transgenic plants with desired traits. In some instances, it is desirable to delay expression of a transgene until a certain developmental stage is reached or environmental condition is encountered. Such transgenes can confer a desired trait or can serve as a selectable marker to aid in the identification of transgenic plants that have been successfully engineered with a polynucleotide of interest.

For example, herbicide tolerance polynucleotides, which encode polypeptides that confer tolerance to specific herbicides, can be introduced into a plant to generate a herbicide tolerant plant and/or to serve as a selectable marker for the introduction of another polynucleotide of interest. Direct selection with herbicides, such as glyphosate and sulfonylureas, during early stages of transgenic plant production (i.e., tissue proliferation) has been relatively inefficient when transforming maize and sugarcane (Experimental Example 1 and unpublished data). Larger clusters of maize cells may be less sensitive to herbicides such as glyphosate and some nontransgenic calli may still grow in the presence of the herbicide (Wang et al. (2009) Handbook of Maize: Genetics and Genomics, J. L. Bennetzen and S. Hake, eds., pp. 609-639). As observed in wheat, however, selection at the stage of regeneration was more effective and escapes were rarely regenerated (Zhou et al. (1995) Plant Cell Rep 15:159-163; Hu et al. (2003) Plant Cell Rep 21:1010-1019).

Thus, methods and compositions are needed that allow for the delayed expression of transgenes to reduce the potential for negative effects on transformed tissues, particularly during development. Such methods and compositions would be especially useful for delaying the expression of herbicide tolerance polynucleotides until a stage at which herbicide selection is more efficient.

### BRIEF SUMMARY OF THE INVENTION

Compositions and methods are provided for the production and selection of transgenic plants and plant parts, for increasing the transformation frequency of a plant or plant part, and for regulating the expression of a transgene, such as a herbicide tolerance polynucleotide. The methods and compositions allow for the delay of the expression of a transgene (e.g., herbicide tolerance polynucleotide) by the presence and subsequent excision of an excision cassette that separates the transgene (e.g., herbicide tolerance polynucleotide) from a promoter that drives its expression. Excision of the excision cassette is mediated by a site-specific recombinase, the expression of which is regulated by an inducible promoter, which results in the operable linkage of the transgene (e.g., herbicide tolerance polynucleotide) and its promoter and subsequent expression of the transgene (e.g., herbicide tolerance polynucleotide). These methods and compositions are useful for delaying the expression of transgenes that might otherwise negatively affect the development or growth of a transformed tissue or plant.

The herbicide tolerance polynucleotide can serve as a means for imparting herbicide tolerance to a plant or plant part and/or can function as a selectable marker, aiding in the identification of a transgenic plant or plant part comprising another polynucleotide of interest or lacking a polynucleotide of interest that has been excised from the excision cassette. In some of these embodiments, the excision of the excision cassette and expression of the herbicide tolerance polynucleotide is delayed until after the tissue proliferation stage of transgenic plant production to allow for more efficient herbicide selection.

In some embodiments, the inducible promoter regulating the expression of the recombinase, excision of the excision cassette, and expression of the herbicide tolerance polynucleotide is one that is induced by stress (e.g., cold temperatures, desiccation) or by a chemical (e.g., antibiotic, herbicide).

Compositions include polynucleotide constructs comprising a promoter that is active in a plant, a herbicide tolerance polynucleotide, and an excision cassette, wherein the excision cassette comprises an inducible promoter operably linked to a site-specific recombinase-encoding polynucleotide, and wherein excision of the excision cassette allows for the operable linkage of the promoter and the herbicide tolerance polynucleotide. Host cells, such as plant cells, and plants and plant parts comprising the polynucleotide constructs are further provided.

The following embodiments are encompassed by the present invention.

1. A polynucleotide construct comprising:
a) an excision cassette comprising an expression cassette A (EC_{A}) comprising:
   i) a promoter A (P_{A}), wherein said P_{A} is an inducible promoter; and
   ii) a coding polynucleotide A (CP_{A}) encoding a site-specific recombinase;

wherein said P_{A} is operably linked to said CP_{A}; and

wherein said excision cassette is flanked by a first and a second recombination site, wherein said first and said second recombination sites are recombinogenic with respect to one another and are directly repeated, and wherein said site-specific recombinase can recognize and implement recombination at said first and said second recombination sites; thereby excising said excision cassette;
b) a coding polynucleotide B (CP_{B}) encoding a herbicide tolerance polypeptide; and
c) a promoter B (P_{B}), wherein said P_{B} is operably linked to said CP_{B} after excision of said excision cassette;

wherein said P_{A} and P_{B} are active in a plant cell.

2. The polynucleotide construct of embodiment 1, wherein said inducible promoter is selected from the group consisting of a stress-inducible promoter and a chemical-inducible promoter.

3. The polynucleotide construct of embodiment 2, wherein said chemical-inducible promoter comprises a promoter comprising a tet operator.

4. The polynucleotide construct of embodiment 3, wherein said polynucleotide construct further comprises a coding polynucleotide F (CP_{F}) encoding a sulfonylurea-responsive transcriptional repressor protein, wherein said CP_{F} is operably linked to a promoter active in a plant cell.

5. The polynucleotide construct of embodiment 2, wherein said stress-inducible promoter can be induced in response to cold, drought, high salinity, desiccation, or a combination thereof

6. The polynucleotide construct of embodiment 2 or 5, wherein said stress-inducible promoter is a maize rab17 promoter or an active variant or fragment thereof

7. The polynucleotide construct of any one of embodiments 2, 5 and 6, wherein said stress-inducible promoter has a nucleotide sequence selected from the group consisting of:
a) the nucleotide sequence having the sequence set forth in SEQ ID NO: 18;
b) a nucleotide sequence having at least 70% sequence identity to the sequence set forth in SEQ ID NO: 18;
c) a nucleotide sequence comprising at least 50 contiguous nucleotides of the sequence set forth in SEQ ID NO: 18;
d) the nucleotide sequence set forth in nucleotides 291-430 of SEQ ID NO: 18; and
e) a nucleotide sequence having at least 70% sequence identity to the sequence set forth in nucleotides 291-430 of SEQ ID NO: 18.

8. The polynucleotide construct of embodiment 6 or 7, wherein said EC_{A} further comprises an attachment B (attB) site between said stress-inducible promoter and said CP_{A}.

9. The polynucleotide construct of embodiment 8, wherein said attB site has a nucleotide sequence selected from the group consisting of:
a) a nucleotide sequence having at least 70% sequence identity to the sequence set forth in SEQ ID NO: 20; and
b) the nucleotide sequence set forth in SEQ ID NO: 20.

10. The polynucleotide construct of any one of embodiments 1-9, wherein said site-specific recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153.

11. The polynucleotide construct of any one of embodiments 1-10, wherein said CP_{A} has the nucleotide sequence selected from the group consisting of:
a) the nucleotide sequence set forth in SEQ ID NO: 33 or 35;
b) a nucleotide sequence having at least 70% sequence identity to SEQ ID NO: 33 or 35;
c) a nucleotide sequence encoding a polypeptide having the amino acid sequence set forth in SEQ ID NO: 34 or 36; and

d) a nucleotide sequence encoding a polypeptide having an amino acid sequence having at least 70% sequence identity to SEQ ID NO: 34 or 36.

12. The polynucleotide construct of any one of embodiments 1-11, wherein P_{B} is a constitutive promoter.

13. The polynucleotide construct of embodiment 12, wherein said P_{B} is selected from the group consisting of a ubiquitin promoter, an oleosin promoter, an actin promoter, and a Mirabilis mosaic virus (MMV) promoter.

14. The polynucleotide construct of any one of embodiments 1-13, wherein said excision cassette further comprises a coding polynucleotide C(CP_{C}) encoding a selectable marker, wherein said CP_{C} is operably linked to a promoter active in a plant cell.

15. The polynucleotide construct of embodiment 14, wherein said CP_{C} is operably linked to P_{B} before excision of the excision cassette.

16. The polynucleotide construct of embodiment 14, wherein said excision cassette further comprises a promoter C(P_{C}), wherein P_{C} is operably linked to said CP_{C}.

17. The polynucleotide construct of embodiment 16, wherein said P_{C} is a constitutive promoter.

18. The polynucleotide construct of embodiment 17, wherein said P_{C} is selected from the group consisting of an ubiquitin promoter, an oleosin promoter, an actin promoter, and a Mirabilis mosaic virus (MMV) promoter.

19. The polynucleotide construct of any one of embodiments 14-18, wherein said selectable marker is selected from the group consisting of a fluorescent protein, an antibiotic resistance polypeptide, a herbicide tolerance polypeptide, and a metabolic enzyme.

20. The polynucleotide construct of embodiment 19, wherein said fluorescent protein is selected from the group consisting of a yellow fluorescent protein, a red fluorescent protein, a cyan fluorescent protein, and a green fluorescent protein.

21. The polynucleotide construct of embodiment 19, wherein said fluorescent protein comprises a Discosoma red fluorescent protein.

22. The polynucleotide construct of embodiment 19, wherein said antibiotic resistance polypeptide comprises a neomycin phosphotransferase II.

23. The polynucleotide construct of embodiment 19, wherein said herbicide tolerance polypeptide encoded by CP_{C} comprises a phosphinothricin acetyl transferase.

24. The polynucleotide construct of embodiment 19, wherein said metabolic enzyme comprises a phosphomannose isomerase.

25. The polynucleotide construct of any one of embodiments 14-24, wherein said excision cassette comprises more than one polynucleotide encoding a distinct selectable marker, wherein said polynucleotide encoding a selectable marker is operably linked to a promoter active in a plant cell.

26. The polynucleotide construct of embodiment 25, wherein said excision cassette comprises at least a first and a second polynucleotide encoding a selectable marker, wherein said first polynucleotide encodes a yellow fluorescent protein, and wherein said second polynucleotide encodes a phosphinothricin acetyl transferase or a neomycin phosphotransferase II.

27. The polynucleotide construct of any one of embodiments 1-26, wherein said herbicide tolerance polypeptide encoded by CP_{B} confers tolerance to a herbicide selected from the group consisting of glyphosate, an ALS inhibitor, an acetyl Co-A carboxylase inhibitor, a synthetic auxin, a protoporphyrinogen oxidase (PPO) inhibitor herbicide, a pigment synthesis inhibitor herbicide, a phosphinothricin acetyltransferase, a phytoene desaturase inhibitor, a glutamine synthase inhibitor, a hydroxyphenylpyruvatedioxygenase inhibitor, and a protoporphyrinogen oxidase inhibitor.

28. The polynucleotide construct of embodiment 27, wherein said ALS inhibitor is selected from the group consisting of a sulfonylurea, a triazolopyrimidine, a pyrimidinyloxy(thio)benzoate, an imidazolinone, and a sulfonylaminocarbonyltriazolinone.

29. The polynucleotide construct of any one of embodiments 1-28, wherein said herbicide tolerance polypeptide encoded by CP_{B} comprises a glyphosate-N-acetyltransferase (GLYAT) polypeptide or an ALS inhibitor-tolerance polypeptide.

30. The polynucleotide construct of embodiment 29, wherein said polynucleotide encoding said GLYAT polypeptide has a nucleotide sequence selected from the group consisting of:
a) the nucleotide sequence set forth in SEQ ID NO: 47 or 49;
b) a nucleotide sequence having at least 95% sequence identity to SEQ ID NO: 47 or 49;
c) a nucleotide sequence encoding a polypeptide having the amino acid sequence set forth in SEQ ID NO: 48 or 50; and
d) a nucleotide sequence encoding a polypeptide having an amino acid sequence having at least 95% sequence identity to SEQ ID NO: 48 or 50.

31. The polynucleotide construct of embodiment 29, wherein said ALS inhibitor-tolerance polypeptide comprises the highly resistant ALS (HRA) mutation of acetolactate synthase.

32. The polynucleotide constructs of any one of embodiments 1-31, wherein said polynucleotide construct comprises more than one polynucleotide encoding a distinct herbicide tolerance polypeptide, wherein the polynucleotide encoding a herbicide tolerance polypeptide is operably linked to a promoter active in a plant cell.

33. The polynucleotide construct of embodiment 32, wherein said polynucleotide construct comprises at least a first and a second polynucleotide encoding a herbicide tolerance polypeptide, wherein said first polynucleotide encodes an ALS inhibitor-tolerance polypeptide and wherein said second polynucleotide encodes a GLYAT polypeptide.

34. The polynucleotide construct of any one of embodiments 1-33, wherein said excision cassette further comprises a coding polynucleotide D (CP_{D}) encoding a cell proliferation factor, wherein said CP_{D} is operably linked to a promoter active in a plant cell.

35. The polynucleotide construct of embodiment 34, wherein said cell proliferation factor is selected from the group consisting of a Lec1 polypeptide, a Kn1 polypeptide, a WUSCHEL polypeptide, a Zwille polypeptide, a babyboom polypeptide, an Aintegumenta polypeptide (ANT), a FUS3 polypeptide, a Kn1polypeptide, a STM polypeptide, an OSH1 polypeptide, and a SbH1 polypeptide.

36. The polynucleotide construct of embodiment 35, wherein said cell proliferation factor is selected from the group consisting of a WUSCHEL polypeptide and a babyboom polypeptide.

37. The polynucleotide construct of any one of embodiments 34-36, wherein said babyboom polypeptide comprises at least two AP2 domains and at least one of the following amino acid sequences:
a) the amino acid sequence set forth in SEQ ID NO: 67 or an amino acid sequence that differs from the amino acid sequence set forth in SEQ ID NO: 67 by one amino acid; and
b) the amino acid sequence set forth in SEQ ID NO: 68 or an amino acid sequence that differs from the amino acid sequence set forth in SEQ ID NO: 68 by one amino acid.

38. The polynucleotide construct of any one of embodiments 34-36, wherein said CP_{D} has a nucleotide sequence selected, from the group consisting of:
a) the nucleotide sequence set forth in SEQ ID NO: 55, 57, 58, 60, 74, 76, 78, 80, 82, 84, 86, 87, 88, 90, 92, 94, 96, 98, 99, or 101;
b) a nucleotide sequence having at least 70% sequence identity to SEQ ID NO: 55, 57, 58, 60, 74, 76, 78, 80, 82, 84, 86, 87, 88, 90, 92, 94, 96, 98, 99, or 101;
c) a nucleotide sequence encoding a polypeptide having the amino acid sequence set forth in SEQ ID NO: 56, 59, 75, 77, 79, 81, 83, 85, 89, 91, 93, 95, 97, 100, or 102; and
d) a nucleotide sequence encoding a polypeptide having an amino acid sequence having at least 70% sequence identity to the amino acid sequence set forth in SEQ ID NO: 56, 59, 75, 77, 79, 81, 83, 85, 89, 91, 93, 95, 97, 100, or 102.

39. The polynucleotide construct of any one of embodiments 34-38, wherein said excision cassette further comprises a promoter D (P_{D}) operably linked to said CP_{D}.

40. The polynucleotide construct of embodiment 39, wherein said P_{D} is a constitutive promoter.

41. The polynucleotide construct of embodiment 40, wherein said P_{D} is a ubiquitin promoter or an oleosin promoter.

42. The polynucleotide construct of any one of embodiments 36-41, wherein said excision cassette comprises more than one coding polynucleotide D (CP_{D}) encoding a distinct cell proliferation factor, wherein the CP_{D} is operably linked to a promoter active in a plant cell.

43. The polynucleotide construct of embodiment 42, wherein said excision cassette comprises at least a first coding polynucleotide D (CP_{D1}) encoding a babyboom polypeptide and a second coding polynucleotide D (CP_{D2}) encoding a WUSCHEL polypeptide.

44. The polynucleotide construct of any one of embodiments 35, 36, 42, and 43, wherein said polynucleotide encoding a WUSCHEL polypeptide has a nucleotide sequence selected from the group consisting of:
a) the nucleotide sequence set forth in SEQ ID NO: 103, 105, 107, or 109; and
b) a nucleotide sequence having at least 70% sequence identity to SEQ ID NO: 103, 105, 107, or 109;
c) a nucleotide sequence encoding a polypeptide having the amino acid sequence set forth in SEQ ID NO: 104, 106, 108, or 110; and
d) a nucleotide sequence encoding a polypeptide having an amino acid sequence having at least 70% sequence identity to SEQ ID NO: 104, 106, 108, or 110.

45. The polynucleotide construct of any one of embodiments 35, 36, 42, 43, and 44, wherein said polynucleotide encoding a WUSCHEL polypeptide is operably linked to a maize In2-2 promoter or a nopaline synthase promoter.

46. The polynucleotide construct of any one of embodiments 1-45, wherein said polynucleotide construct further comprises a coding polynucleotide E (CP_{E}) encoding a polypeptide of interest, wherein said CP_{E} is operably linked to a promoter active in a plant cell.

47. The polynucleotide construct of embodiment 46, wherein said excision cassette comprises said CP_{E}.

48. The polynucleotide construct of embodiment 46, wherein said CP_{E} is outside of the excision cassette.

49. The polynucleotide construct of any one of embodiments 46-48, wherein said polynucleotide construct further comprises a promoter E (P_{E}) operably linked to said CP_{E}.

50. The polynucleotide construct of embodiment 1, wherein said polynucleotide construct comprises:
a) a first ubiquitin promoter;
b) an excision cassette flanked by loxP recombination sites that are are recombinogenic with respect to one another and are directly repeated, wherein said excision cassette comprises:
   i) a polynucleotide encoding a phosphinothricin acetyl transferase (PAT) or a neomycin phosphotransferase II (NPTII);
   ii) a second ubiquitin promoter;
   iii) a polynucleotide encoding a yellow fluorescent protein;
   iv) a promoter comprising a maize rab17 promoter and an attachment B (attB) site;
   v) a polynucleotide encoding a CRE recombinase;
   vi) a nopaline synthase promoter;
   vii) a polynucleotide encoding a maize Wuschel 2 polypeptide;
   viii) a third ubiquitin promoter; and
   ix) a babyboom polynucleotide; and
c) a GLYAT polynucleotide;

wherein said first ubiquitin promoter is operably linked to said polynucleotide encoding said PAT or NPTII and wherein said first ubiquitin promoter is operably linked to said GLYAT polynucleotide upon excision of said excision cassette;

wherein said second ubiquitin promoter is operably linked to said polynucleotide encoding said yellow fluorescent protein;

wherein said promoter comprising said maize rab17 promoter and said attB site is operably linked to said polynucleotide encoding said CRE recombinase;

wherein said nopaline synthase promoter is operably linked to said polynucleotide encoding said maize Wuschel 2 polypeptide;

and wherein said third ubiquitin promoter is operably linked to said babyboom polynucleotide.

51. The polynucleotide construct of embodiment 1, wherein said polynucleotide construct comprises:
a) a ubiquitin promoter;
b) an excision cassette flanked by loxP recombination sites that are are recombinogenic with respect to one another and are directly repeated, wherein said excision cassette comprises:
   i) a polynucleotide encoding a Discosoma red fluorescent protein;
   ii) a promoter comprising a maize rab17 promoter and an attachment B (attB) site; and
   iii) a polynucleotide encoding a CRE recombinase; and
c) a GLYAT polynucleotide;

wherein said ubiquitin promoter is operably linked to said polynucleotide encoding said Discosoma red fluorescent protein and wherein said ubiquitin promoter is operably linked to said GLYAT polynucleotide upon excision of said excision cassette; and

wherein said promoter comprising said maize rab17 promoter and said attB site is operably linked to said polynucleotide encoding said CRE recombinase.

52. The polynucleotide construct of embodiment 1, wherein said polynucleotide construct comprises:
a) a ubiquitin promoter;
b) an excision cassette flanked by loxP recombination sites that are are recombinogenic with respect to one another and are directly repeated, wherein said excision cassette comprises:
   i) an actin promoter;
   ii) a polynucleotide encoding a Discosoma red fluorescent protein;
   iii) a promoter comprising a maize rab17 promoter and an attachment B (attB) site; and
   iv) a polynucleotide encoding a CRE recombinase; and
c) a GLYAT polynucleotide;

wherein said ubiquitin promoter is operably linked to said GLYAT polynucleotide upon excision of said excision cassette;

wherein said actin promoter is operably linked to said polynucleotide encoding said Discosoma red fluorescent protein; and
wherein said promoter comprising said maize rab17 promoter and said attB site is operably linked to said polynucleotide encoding said CRE recombinase.

53. A host cell comprising the polynucleotide construct of any one of embodiments 1-52.

54. A plant cell comprising the polynucleotide construct of any one of embodiments 1-52.

55. A plant or plant part comprising said plant cell of embodiment 54.

56. The plant or plant part of embodiment 55, wherein said plant or plant part is a dicot.

57. The plant or plant part of embodiment 55, wherein said plant or plant part is a monocot.

58. The plant or plant part of embodiment 57, wherein said monocot is selected, from the group consisting of maize, rice, sorghum, barley, wheat, millet, oat, rye, triticale, sugarcane, switchgrass, and turf/forage grass.

59. The plant or plant part of any one of embodiments 55-58, wherein said plant or plant part is recalcitrant.

60. The plant or plant part of embodiment 59, wherein said plant or plant part is a sugarcane cultivar selected from the group consisting of CP96-1252, CP01-1372, CPCL97-2730, HoCP85-845, CP89-2143, and KQ228.

61. The plant or plant part of any one of embodiments 55-60, wherein said plant part is a seed.

62. A method for producing a transgenic plant or plant part, said method comprising introducing said polynucleotide construct of any one of embodiments 1-52 into a plant or plant part.

63. A method for regulating the expression of a herbicide tolerance polynucleotide, wherein said method comprises:
a) providing the host cell of embodiment 53, the plant cell of embodiment 54, or the plant or plant part of any one of embodiments 55-61; and,
b) inducing the expression of said site-specific recombinase, thereby excising said excision cassette from said polynucleotide construct and expressing said herbicide tolerance polynucleotide.

64. A method for selecting a herbicide tolerant plant cell, said method comprising the steps of:
A) providing a population of plant cells, wherein at least one plant cell in the population comprises a polynucleotide construct comprising:
a) an excision cassette comprising an expression cassette A (EC_{A}) comprising:
   i) a promoter A (P_{A}), wherein said P_{A} is an inducible promoter; and
   ii) a coding polynucleotide A (CP_{A}) encoding a site-specific recombinase;

wherein said P_{A} is operably linked to said CP_{A};
b) a coding polynucleotide B (CP_{B}) encoding a herbicide tolerance polypeptide; and
c) a promoter B (P_{B}), wherein said P_{B} is operably linked to said CP_{B} after excision of said excision cassette;

wherein said P_{A} and P_{B} are active in a plant cell; and

wherein said excision cassette is flanked by a first and a second recombination site, wherein said first and said second recombination sites are recombinogenic with respect to one another and are directly repeated, and wherein said site-specific recombinase can recognize and implement recombination at said first and said second recombination sites; thereby excising said excision cassette;
B) inducing the expression of said site-specific recombinase; and
C) contacting said population of plant cells with a herbicide to which said herbicide tolerance polypeptide confers tolerance, thereby selecting for a plant cell having tolerance to said herbicide.

65. The method of embodiment 64, wherein said provided population of plant cells is cultured into a population of plant tissues or plants prior to, during, or after said step B), and wherein said step C) comprises contacting said population of plant tissues or plants with said herbicide.

66. The method of embodiment 65, wherein said step C) occurs during or after regeneration of said provided population of plant cells into a population of plants.

67. The method of embodiment 64, wherein said provided population of plant cells is a population of immature or mature seeds, wherein at least one immature or mature seed within said population of immature or mature seeds comprises said polynucleotide construct.

68. The method of embodiment 67, wherein said provided population of seeds is planted prior to, during, or after said step B) to produce a population of plants, and wherein said step C) comprises contacting said population of plants with said herbicide.

69. The method of embodiment 75, wherein said provided population of plant cells is a population of plant tissues, wherein at least one plant tissue within said population of plant tissues comprises said polynucleotide construct.

70. The method of embodiment 69, wherein said provided population of plant tissues is cultured into a population of plants prior to, during, or after said step B), and wherein said step C) comprises contacting said population of plants with said herbicide.

71. The method of embodiment 64, wherein said provided population of plant cells is a population of plants, wherein at least one plant within said population of plants comprises said polynucleotide construct.

72. The method of any one of embodiments 64-71, wherein said method further comprises introducing said polynucleotide construct into said at least one plant cell before step A).

73. The method of any one of embodiments 64-72, wherein said inducible promoter P_{A} is selected from the group consisting of a stress-inducible promoter and a chemical-inducible promoter.

74. The method of embodiment 73, wherein said chemical-inducible promoter comprises a promoter comprising a tet operator.

75. The method of embodiment 74, wherein said polynucleotide construct or said at least one plant cell further comprises a coding polynucleotide F (CP_{F}) encoding a sulfonylurea-responsive transcriptional repressor protein, wherein said CP_{F} is operably linked to a promoter active in a plant cell, and wherein said inducing comprises contacting said population of plant cells with a sulfonylurea compound.

76. The method of embodiment 73, wherein said stress-inducible promoter is induced in response to cold, drought, desiccation, high salinity, or a combination thereof

77. The method of embodiment 73 or 76, wherein said stress-inducible promoter comprises a drought-inducible promoter, and wherein said inducing comprises desiccating said population of plant cells.

78. The method of embodiment 77, wherein said desiccating occurs during the maturation of an immature seed.

79. The method of embodiment 73, wherein said stress-inducible promoter is a maize rab17 promoter or an active variant or fragment thereof.

80. The method of embodiment 73, wherein said stress-inducible promoter has a nucleotide sequence selected from the group consisting of:
a) the nucleotide sequence having the sequence set forth in SEQ ID NO: 18;
b) a nucleotide sequence having at least 70% sequence identity to the sequence set forth in SEQ ID NO: 18;
c) a nucleotide sequence comprising at least 50 contiguous nucleotides of the sequence set forth in SEQ ID NO: 18;
d) the nucleotide sequence set forth in nucleotides 291-430 of SEQ ID NO: 18; and
e) a nucleotide sequence having at least 70% sequence identity to the sequence set forth in nucleotides 291-430 of SEQ ID NO: 18.

81. The method of embodiment 79 or 80, wherein said EC_{A} further comprises an attachment B (attB) site between said stress-inducible promoter and said CP_{A}.

82. The method of embodiment 81, wherein said attB site has a nucleotide sequence selected from the group consisting of:
a) a nucleotide sequence having at least 70% sequence identity to the sequence set forth in SEQ ID NO: 20; and
b) the nucleotide sequence set forth in SEQ ID NO: 20.

83. The method of any one of embodiments 64-82, wherein said site-specific recombinase is selected from the group consisting of FLP, Cre, S-CRE, V-CRE, Dre, SSV1, lambda Int, phi C31 Int, HK022, R, Gin, Tn1721, CinH, ParA, Tn5053, Bxb1, TP907-1, and U153.

84. The method of any one of embodiments 64-83, wherein said CP_{A} has the nucleotide sequence selected from the group consisting of:
a) the nucleotide sequence set forth in SEQ ID NO: 33 or 35;
b) a nucleotide sequence having at least 70% sequence identity to SEQ ID NO: 33 or 35;
c) a nucleotide sequence encoding a polypeptide having the amino acid sequence set forth in SEQ ID NO: 34 or 36; and
d) a nucleotide sequence encoding a polypeptide having an amino acid sequence having at least 70% sequence identity to SEQ ID NO: 34 or 36.

85. The method of any one of embodiments 64-84, wherein P_{B} is a constitutive promoter.

86. The method of embodiment 85, wherein said P_{B} is selected from the group consisting of a ubiquitin promoter, an oleosin promoter, an actin promoter, and a Mirabilis mosaic virus promoter.

87. The method of any one of embodiments 64-86, wherein said excision cassette further comprises a coding polynucleotide C(CP_{C}), wherein said CP_{C} encodes a selectable marker, wherein said CP_{C} is operably linked to a promoter active in a plant cell, and wherein said method further comprises a selection step prior to step B), wherein those plant cells within said population of plant cells that comprise said selectable marker are identified and wherein these selected plant cells comprise the population of plant cells that are induced in step B).

88. The method of embodiment 87, wherein said CP_{C} is operably linked to P_{B}.

89. The method of embodiment 87, wherein said excision cassette further comprises a promoter C(P_{C}), wherein P_{C} is operably linked to said CP_{C}.

90. The method of embodiment 89, wherein P_{C} is a constitutive promoter.

91. The method of embodiment 90, wherein said P_{C} is selected from the group consisting of a ubiquitin promoter, an oleosin promoter, an actin promoter, and a Mirabilis mosaic virus promoter.

92. The method of any one of embodiments 87-91, wherein said selectable marker is selected from the group consisting of a fluorescent protein, an antibiotic resistance polypeptide, a herbicide tolerance polypeptide, and a metabolic enzyme.

93. The method of embodiment 92, wherein said fluorescent protein is selected from the group consisting of a yellow fluorescent protein, a red fluorescent protein, a cyan fluorescent protein, and a green fluorescent protein.

94. The method of embodiment 92, wherein said fluorescent protein comprises a Discosoma red fluorescent protein.

95. The method of embodiment 92, wherein said antibiotic resistance polypeptide comprises a neomycin phosphotransferase II.

96. The method of embodiment 92, wherein said herbicide tolerance polypeptide encoded by CP_{C} comprises a phosphinothricin acetyl transferase.

97. The method of embodiment 92, wherein said metabolic enzyme comprises a phosphomannose isomerase.

98. The method of any one of embodiments 87-97, wherein said excision cassette comprises more than one polynucleotide encoding a distinct selectable marker, wherein said polynucleotide encoding a selectable marker is operably linked to a promoter active in a plant cell.

99. The method of embodiment 98, wherein said excision cassette comprises at least a first and a second polynucleotide encoding a selectable marker, wherein said first polynucleotide encodes a yellow fluorescent protein, and wherein said second polynucleotide encodes a phosphinothricin acetyl transferase or a neomycin phosphotransferase II.

100. The method of any one of embodiments 64-99, wherein said herbicide tolerance polypeptide encoded by CP_{B} confers tolerance to a herbicide selected from the group consisting of glyphosate, an ALS inhibitor, an acetyl Co-A carboxylase inhibitor, a synthetic auxin, a protoporphyrinogen oxidase (PPO) inhibitor herbicide, a pigment synthesis inhibitor herbicide, a phosphinothricin acetyltransferase, a phytoene desaturase inhibitor, a glutamine synthase inhibitor, a hydroxyphenylpyruvatedioxygenase inhibitor, and a protoporphyrinogen oxidase inhibitor.

101. The method of embodiment 100, wherein said ALS inhibitor is selected from the group consisting of a sulfonylurea, a triazolopyrimidine, a pyrimidinyloxy(thio)benzoate, an imidazolinone, and a sulfonylaminocarbonyltriazolinone.

102. The method of any one of embodiments 64-101, wherein said herbicide tolerance polypeptide encoded by CP_{B} comprises a glyphosate-N-acetyltransferase (GLYAT) polypeptide or an ALS inhibitor-tolerance polypeptide.

103. The method of embodiment 102, wherein said polynucleotide encoding said GLYAT polypeptide has a nucleotide sequence selected from the group consisting of:
a) the nucleotide sequence set forth in SEQ ID NO: 47 or 49;
b) a nucleotide sequence having at least 95% sequence identity to SEQ ID NO: 47 or 49;
c) a nucleotide sequence encoding a polypeptide having the amino acid sequence set forth in SEQ ID NO: 48 or 50; and
d) a nucleotide sequence encoding a polypeptide having an amino acid sequence having at least 95% sequence identity to SEQ ID NO: 48 or 50.

104. The method of embodiment 102, wherein said ALS inhibitor-tolerance polypeptide comprises the highly resistant ALS (HRA) mutation of acetolactate synthase.

105. The method of any one of embodiments 64-104, wherein said polynucleotide construct comprises more than one polynucleotide encoding a distinct herbicide tolerance polypeptide, wherein said polynucleotide encoding a herbicide tolerance polypeptide is operably linked to a promoter active in a plant cell.

106. The method of embodiment 105, wherein said polynucleotide construct comprises at least a first and a second polynucleotide encoding a herbicide tolerance polypeptide, wherein said first polynucleotide encodes an ALS inhibitor-tolerance polypeptide, and wherein said second polynucleotide encodes a GLYAT polypeptide.

107. The method of any one of embodiments 64-106, wherein said excision cassette further comprises a coding polynucleotide D (CP_{D}), wherein said CP_{D} encodes a cell proliferation factor, and wherein said CP_{D} is operably linked to a promoter active in a plant cell.

108. The method of embodiment 107, wherein said cell proliferation factor is selected from the group consisting of a Lec1 polypeptide, a Kn1 polypeptide, a WUSCHEL polypeptide, a Zwille polypeptide, a babyboom polypeptide, an Aintegumenta polypeptide (ANT), a FUS3 polypeptide, a Kn1 polypeptide, a STM polypeptide, an OSH1 polypeptide, and a SbH1 polypeptide.

109. The method of embodiment 108, wherein said cell proliferation factor is selected from the group consisting of a WUSCHEL polypeptide and a babyboom polypeptide.

110. The method of any one of embodiments 107-109, wherein said babyboom polypeptide comprises at least two AP2 domains and at least one of the following amino acid sequences:
a) the amino acid sequence set forth in SEQ ID NO: 67 or an amino acid sequence that differs from the amino acid sequence set forth in SEQ ID NO: 67 by one amino acid; and
b) the amino acid sequence set forth in SEQ ID NO: 68 or an amino acid sequence that differs from the amino acid sequence set forth in SEQ ID NO: 68 by one amino acid.

111. The method of any one of embodiments 107-109, wherein said CP_{D} has a nucleotide sequence selected from the group consisting of:
a) the nucleotide sequence set forth in SEQ ID NO: 55, 57, 58, 60, 74, 76, 78, 80, 82, 84, 86, 87, 88, 90, 92, 94, 96, 98, 99, or 101;
b) a nucleotide sequence having at least 70% sequence identity to SEQ ID NO: 55, 57, 58, 60, 74, 76, 78, 80, 82, 84, 86, 87, 88, 90, 92, 94, 96, 98, 99, or 101;
c) a nucleotide sequence encoding a polypeptide having the amino acid sequence set forth in SEQ ID NO: 56, 59, 75, 77, 79, 81, 83, 85, 89, 91, 93, 95, 97, 100, or 102; and
d) a nucleotide sequence encoding a polypeptide having an amino acid sequence having at least 70% sequence identity to the amino acid sequence set forth in SEQ ID NO: 56, 59, 75, 77, 79 81, 83, 85, 89, 91, 93, 95, 97, 100, or 102.

112. The method of any one of embodiments 107-111, wherein said excision cassette further comprises a promoter D (P_{D}), wherein said P_{D} is operably linked to said CP_{D}.

113. The method of embodiment 112, wherein said P_{D} is a constitutive promoter.

114. The method of embodiment 112 or 113, wherein said P_{D} is an ubiquitin promoter or an oleosin promoter.

115. The method of any one of embodiments 107-114, wherein said excision cassette comprises more than one polynucleotide encoding a distinct cell proliferation factor, wherein the polynucleotide encoding a cell proliferation factor is operably linked to a promoter active in a plant cell.

116. The method of embodiment 115, wherein said excision cassette comprises at least a first coding polynucleotide D (CP_{D1}) encoding a babyboom polypeptide and a second coding polynucleotide D (CP_{D2}) encoding a WUSCHEL polypeptide.

117. The method of any one of embodiments 108, 109, and 116, wherein said polynucleotide encoding a WUSCHEL polypeptide has a nucleotide sequence selected from the group consisting of:
a) the nucleotide sequence set forth in SEQ ID NO: 103, 105, 107, or 109; and
b) a nucleotide sequence having at least 70% sequence identity to SEQ ID NO: 103, 105, 107, or 109;
c) a nucleotide sequence encoding a polypeptide having the amino acid sequence set forth in SEQ ID NO: 104, 106, 108, or 110; and
d) a nucleotide sequence encoding a polypeptide having an amino acid sequence having at least 70% sequence identity to SEQ ID NO: 104, 106, 108, or 110.

118. The method of any one of embodiments 108, 109, 116, and 117, wherein said polynucleotide encoding a WUSCHEL polypeptide is operably linked to a maize In2-2 promoter or a nopaline synthase promoter.

119. The method of any one of embodiments 64-118, wherein said polynucleotide construct further comprises a coding polynucleotide E (CP_{E}) encoding a polypeptide of interest, wherein the CP_{E} is operably linked to a promoter active in a plant cell.

120. The method of embodiment 119, wherein said excision cassette comprises said CP_{E}, and wherein said selected herbicide tolerant plant cell lacks said CP_{E}.

121. The method of embodiment 119, wherein said CP_{E} is outside of the excision cassette, and wherein said selected herbicide tolerant plant cell comprises said CP_{E}.

122. The method of any one of embodiments 119-121, wherein said polynucleotide construct further comprises a promoter E (P_{E}) operably linked to said CP_{E}.

123. The method of embodiment 64, wherein said polynucleotide construct comprises:
a) a first ubiquitin promoter;
b) an excision cassette flanked by loxP recombination sites that are are recombinogenic with respect to one another and are directly repeated, wherein said excision cassette comprises:
   i) a polynucleotide encoding a phosphinothricin acetyl transferase (PAT) or a neomycin phosphotransferase II (NPTII);
   ii) a second ubiquitin promoter;
   iii) a polynucleotide encoding a yellow fluorescent protein;
   iv) a promoter comprising a maize rab17 promoter and an attachment B (attB) site;
   v) a polynucleotide encoding a CRE recombinase;
   vi) a nopaline synthase promoter;
   vii) a polynucleotide encoding a maize Wuschel 2 polypeptide;
   viii) a third ubiquitin promoter; and
   ix) a babyboom polynucleotide; and
c) a GLYAT polynucleotide;

wherein said first ubiquitin promoter is operably linked to said polynucleotide encoding said PAT or NPTII and wherein said first ubiquitin promoter is operably linked to said GLYAT polynucleotide upon excision of said excision cassette;

wherein said second ubiquitin promoter is operably linked to said polynucleotide encoding said yellow fluorescent protein;

wherein said promoter comprising said maize rab17 promoter and said attB site is operably linked to said polynucleotide encoding said CRE recombinase;

wherein said nopaline synthase promoter is operably linked to said polynucleotide encoding said maize Wuschel 2 polypeptide;

and wherein said third ubiquitin promoter is operably linked to said babyboom polynucleotide.

124. The method of embodiment 64, wherein said polynucleotide construct comprises:
a) a ubiquitin promoter;
b) an excision cassette flanked by loxP recombination sites that are are recombinogenic with respect to one another and are directly repeated, wherein said excision cassette comprises:
   i) a polynucleotide encoding a Discosoma red fluorescent protein;
   ii) a promoter comprising a maize rab17 promoter and an attachment B (attB) site; and
   iii) a polynucleotide encoding a CRE recombinase; and
c) a GLYAT polynucleotide;

wherein said ubiquitin promoter is operably linked to said polynucleotide encoding said Discosoma red fluorescent protein and wherein said ubiquitin promoter is operably linked to said GLYAT polynucleotide upon excision of said excision cassette; and

wherein said promoter comprising said maize rab17 promoter and said attB site is operably linked to said polynucleotide encoding said CRE recombinase.

125. The method of embodiment 64, wherein said polynucleotide construct comprises:
a) a ubiquitin promoter;
b) an excision cassette flanked by loxP recombination sites that are are recombinogenic with respect to one another and are directly repeated, wherein said excision cassette comprises:
   i) an actin promoter;
   ii) a polynucleotide encoding a Discosoma red fluorescent protein;
   iii) a promoter comprising a maize rab17 promoter and an attachment B (attB) site; and
   iv) a polynucleotide encoding a CRE recombinase; and
c) a GLYAT polynucleotide;

wherein said ubiquitin promoter is operably linked to said GLYAT polynucleotide upon excision of said excision cassette;

wherein said actin promoter is operably linked to said polynucleotide encoding said Discosoma red fluorescent protein; and

wherein said promoter comprising said maize rab17 promoter and said attB site is operably linked to said polynucleotide encoding said CRE recombinase.

126. The method of any one of embodiments 64-125, wherein said plant cells are dicotyledonous.

127. The method of any one of embodiments 64-125, wherein said plant cells are monocotyledonous.

128. The method of embodiment 127, wherein said monocotyledonous plant cell is selected from the group consisting of maize, rice, sorghum, barley, wheat, millet, oat, rye, triticale, sugarcane, switchgrass, and turf/forage grass.

129. The method of any one of embodiments 64-128, wherein said plant cells are recalcitrant.

130. The method of embodiment 129, wherein said recalcitrant plant cells are cells of a sugarcane cultivar selected from the group consisting of CP96-1252, CP01-1372, CPCL97-2730, HoCP85-845, CP89-2143, and KQ228.

131. A method for increasing the transformation frequency of a plant tissue, the method comprising the steps of:
a) providing a population of plant cells, wherein at least one plant cell in the population comprises the polynucleotide construct of any one of claims 1-52;
b) culturing the population of plant cells in the absence of a herbicide to which the herbicide tolerance polypeptide confers herbicide resistance for a period of time sufficient for the population of plant cells to proliferate;
c) inducing the expression of the site-specific recombinase, thereby excising the excision cassette;
d) contacting the population of plant cells from c) with the herbicide to which the herbicide tolerance polypeptide confers tolerance; and
e) selecting for a plant cell having tolerance to the herbicide, wherein the transformation frequency is increased compared to a comparable plant cell not comprising the excision cassette and selected directly by herbicide selection.

132. The method of embodiment 131, wherein the inducing comprises desiccating the population of plant cells.

133. The method of embodiment 131 or 132, wherein the population of plant cells is cultured in the absence of the herbicide to which the herbicide tolerance polypeptide confers herbicide resistance for about 1 hour to about 6 weeks prior to excision.

### BRIEF DESCRIPTION OF THE FIGURES

FIG. 1 provides a depiction of vector PHP35648. The vector comprises a coding sequence for the cyan fluorescent protein (CFP), the expression of which is regulated by the ubiquitin promoter (Ubi Pro; comprising the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113)). The PHP35648 vector comprises the maize rab17 promoter with an attachment B site (Rab17 Pro) that drives the expression of the CRE site-specific recombinase. The vector further comprises expression cassettes for the maize Wuschel 2 (WUS2) protein (the expression of which is regulated by the nopaline synthase (Nos) promoter), the maize babyboom (BBM) protein and the maize optimized phosphinothricin acetyl transferase (moPAT) (both of which are regulated by the ubiquitin promoter; comprising the maize ubiquitin promoter (Ubi Pro; comprising the UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113)). The yellow fluorescent protein (YFP) is expressed when a fragment of the vector that is flanked by LoxP recombination sites (the excision cassette) is excised by the CRE recombinase.

FIG. 2 provides a depiction of vector PHP54561. The vector comprises a coding sequence for moPAT or neomycin phosphotransferase II (nptII), the expression of which is regulated by the ubiquitin promoter (Ubi Pro; comprising the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113)). An ubiquitin promoter (Ubi Pro) also regulates the expression of yellow fluorescent protein (YFP) and the maize BBM protein. The PHP54561 vector further comprises the maize rab17 promoter with an attachment B site (Rab17 Pro) that drives the expression of the CRE recombinase and an expression cassette for WUS2 under the regulation of the Nos promoter. The ubiquitin promoter (Ubi Pro) regulates the expression of the glyphosate-N-acetyltransferase (GLYAT) gene when an excision cassette flanked by LoxP sites is excised by the CRE recombinase.

FIG. 3 provides an image of glyphosate selection on tissue proliferation/regeneration medium of tissues of sugarcane cultivars CP01-1372 (top) and CP88-1762 (bottom) that had been transformed with the PHP54561 vector and desiccated.

FIG. 4 provides images of glyphosate selection on regeneration/rooting medium of sugarcane cultivars CP01-1372 (left) and CP88-1762 (right) that had been transformed with the PHP54561 vector and desiccated.

FIG. 5 provides images of a second round of glyphosate selection on rooting medium containing 30 μM glyphosate of sugarcane that had been transformed with the PHP54561 vector and desiccated.

FIG. 6 provides a depiction of vector PHP54353. The vector comprises a coding sequence for the red fluorescent protein from Discosoma (dsRED), the expression of which is regulated by the ubiquitin promoter (Ubi Pro; comprising the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113)). The PHP54353 vector comprises the maize rab17 promoter with an attachment B site (Rab17 Pro) that drives the expression of the CRE site-specific recombinase. The ubiquitin promoter (Ubi Pro) regulates the expression of the glyphosate-N-acetyltransferase (GLYAT) gene when an excision cassette flanked by LoxP sites is excised by the CRE recombinase.

FIG. 7 provides a depiction of another polynucleotide construct embodiment. The vector comprises a coding sequence for the red fluorescent protein from Discosoma (dsRED), the expression of which is regulated by the actin promoter (Actin Pro). The vector further comprises the maize rab17 promoter with an attachment B site (Rab17 Pro) that drives the expression of the CRE site-specific recombinase. The ubiquitin promoter (Ubi Pro; comprising the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113) regulates the expression of the glyphosate-N-acetyltransferase (GLYAT) gene when an excision cassette flanked by LoxP sites is excised by the CRE recombinase.

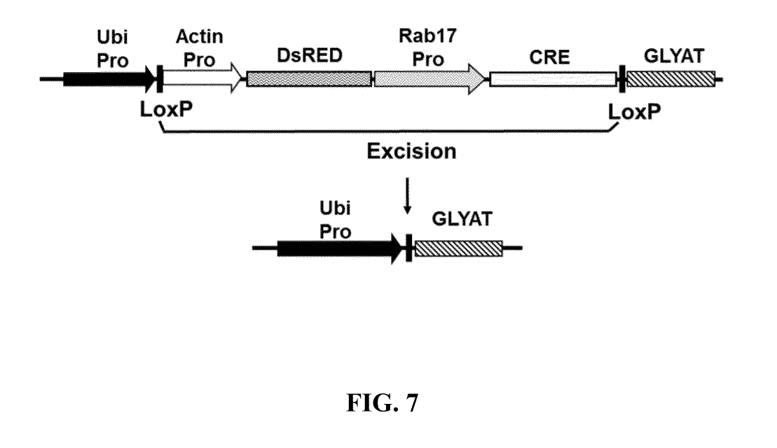

FIG. 8 provides a depiction of vector PHP55062. The vector comprises a coding sequence for the red fluorescent protein from Discosoma (dsRED), the expression of which is regulated by the enhanced Mirabilis mosaic virus (dMMV) promoter. The vector further comprises the maize rab17 promoter with an attachment B site (Rab17 Pro) that drives the expression of the CRE site-specific recombinase. A separate dMMV promoter regulates the expression of a hygromycin phosphotransferase (Hyg (hpt)) gene and also regulates the expression of the glyphosate-N-acetyltransferase (GLYAT) gene when an excision cassette flanked by LoxP sites is excised by the CRE recombinase.

FIG. 9 provides depictions of various embodiments of the presently disclosed polynucleotide constructs. The constructs all comprise an excision cassette (flanked by LoxP sites) comprising a polynucleotide encoding a site-specific recombinase (CP_{A}), the expression of which is regulated by an inducible promoter A (P_{A}). Upon activation of P_{A} and excision of the excision cassette, promoter B (P_{B}) is operably linked to the polynucleotide encoding a herbicide tolerance polypeptide (CP_{B}) and the herbicide tolerance polypeptide is produced. The excision cassette of the constructs of FIGS. 9b-9g further comprise a polynucleotide encoding a selectable marker (CP_{C}) in the excision cassette that is either operably linked to P_{B} or to another promoter (P_{C}). The excision cassettes of the constructs of FIGS. 9d-9g further comprises at least one polynucleotide encoding a cell proliferation factor (CP_{D1} and CP_{D2}), each of which are operably linked to a promoter (P_{D1} or P_{D2}, respectively). The polynucleotide construct of FIG. 9g further comprises (outside of the excision cassette) a polynucleotide encoding a polypeptide of interest (CP_{E}) that is operably linked to a promoter E (P_{E}).

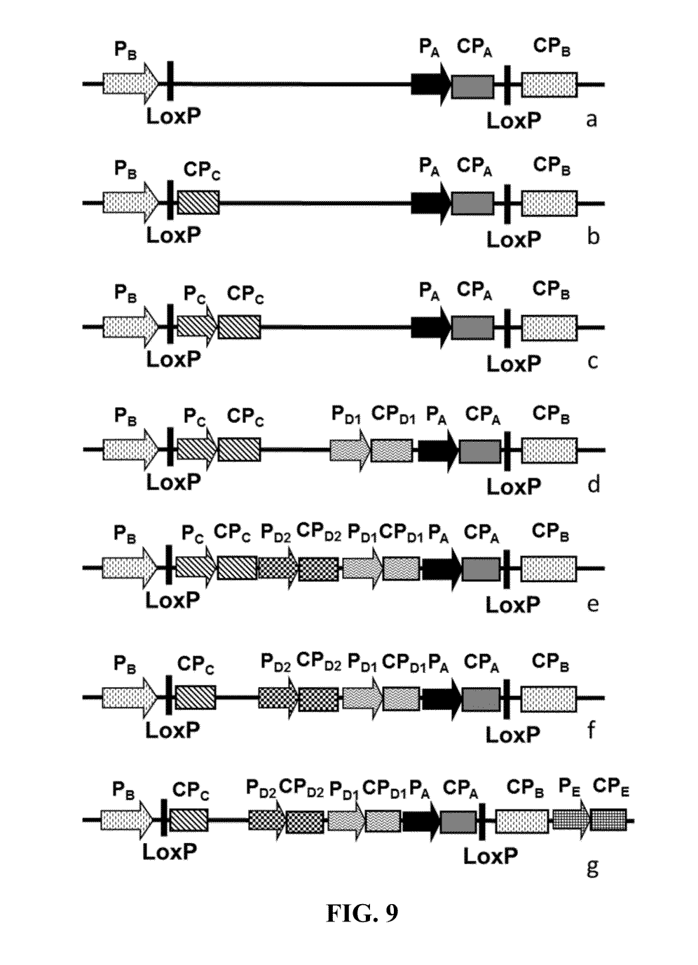

### DETAILED DESCRIPTION OF THE INVENTION

Compositions and methods are provided for regulating the expression of a transgene, such as a herbicide tolerance polynucleotide, for producing and selecting transgenic plants and plant parts, and for increasing the transformation frequency of a plant or plant part. Compositions include polynucleotide constructs comprising an excision cassette, a transgene (e.g., herbicide tolerance polynucleotide) and a promoter that becomes operably linked to the transgene (e.g., herbicide tolerance polynucleotide) upon excision of the excision cassette from the polynucleotide construct. The excision cassette comprises an inducible promoter operably linked to a polynucleotide that encodes a site-specific recombinase and the excision cassette is flanked by a first and a second recombination site, wherein the first and second recombination sites are recombinogenic with respect to one another and are directly repeated, and wherein the site-specific recombinase can recognize and implement recombination at the first and second recombination sites, thereby excising the excision cassette and allowing for the operable linkage of the transgene (e.g., herbicide tolerance polynucleotide) with its promoter. In some embodiments, the polynucleotide construct further comprises a polynucleotide of interest, either within or outside of the excision cassette. In certain embodiments, the excision cassette further comprises at least one coding polynucleotide for a cell proliferation factor, such as a babyboom polypeptide or a Wuschel polypeptide.

In some embodiments, the polynucleotide construct further comprises at least one selectable marker. In some embodiments, the selectable marker is selected from the group consisting of a fluorescent protein, an antibiotic resistance polypeptide, a herbicide tolerance polypeptide, and a metabolic enzyme. In some embodiments, the plant or plant part is recalcitrant to transformation. In some embodiments, the plant or plant part is a monocotyledonous. In some embodiments the plant or plant part is maize, rice, wheat, barley, sorghum, oats, rye, triticale and sugarcane.

It is intended that the excision cassette is not limited by the number and or order of the coding polynucleotides within the excision cassette. It is envisioned that the excision cassette can be constructed with any number of coding polynucleotides in any order. It is also intended that the polynucleotide construct may also include, beyond the promoter and polynucleotide encoding the herbicide tolerance polypeptide flanking the recombination sites, one or more polynucleotide encoding polypeptide(s) of interest.

The use of the term “polynucleotide” is not intended to limit compositions to polynucleotides comprising DNA. Polynucleotides can comprise ribonucleotides and combinations of ribonucleotides and deoxyribonucleotides. Such deoxyribonucleotides and ribonucleotides include both naturally occurring molecules and synthetic analogues. The polynucleotides also encompass all forms of sequences including, but not limited to, single-, double-, or multi-stranded forms, hairpins, stem-and-loop structures, circular plasmids, and the like.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein or biologically active portion thereof is recombinantly produced, optimally culture medium represents less than about 30%, 20%, 10%, 5%, or 1% (by dry weight) of chemical precursors or non-protein-of-interest chemicals.

As used herein, a “polynucleotide construct” refers to a polynucleotide molecule comprised of various types of nucleotide sequences having different functions and/or activities. For example, a polynucleotide construct may comprise one or more of any of the following: expression cassettes, coding polynucleotides, regulatory sequences (e.g., enhancers, promoters, termination sequences), origins of replication, restriction sites, recombination sites, and excision cassettes.

The presently disclosed polynucleotide constructs can comprise one or more expression cassettes, wherein a coding polynucleotide is operably linked to a regulatory sequence.

As used herein, a “coding polynucleotide” refers to a polynucleotide that encodes a polypeptide and therefore comprises the requisite information to direct translation of the nucleotide sequence into a specified polypeptide. Alternatively, a “coding polynucleotide” can refer to a polynucleotide that encodes a silencing polynucleotide that reduces the expression of target genes. Non-limiting examples of a silencing polynucleotide include a small interfering RNA, micro RNA, antisense RNA, a hairpin structure, and the like.

As used herein, an “expression cassette” refers to a polynucleotide that comprises at least one coding polynucleotide operably linked to regulatory sequences sufficient for the expression of the coding polynucleotide. “Operably linked” is intended to mean a functional linkage between two or more elements. For example, an operable linkage between a coding polynucleotide and a regulatory sequence (i.e., a promoter) is a functional link that allows for expression of the coding polynucleotide. Operably linked elements may be contiguous or non-contiguous. When used to refer to the joining of two protein coding regions, by operably linked is intended that the coding regions are in the same reading frame.

An expression cassette will include in the 5′-3′ direction of transcription, a transcriptional and translational initiation region (i.e., a promoter), a coding polynucleotide, and a transcriptional and translational termination region (i.e., termination region) functional in plants. The regulatory regions (i.e., promoters, transcriptional regulatory regions, and translational termination regions) and/or the coding polynucleotide may be native/analogous to a host cell comprising the presently disclosed polynucleotide constructs or to each other. Alternatively, the regulatory regions and/or the coding polynucleotide may be heterologous to the host cell or to each other. As used herein, “heterologous” in reference to a sequence is a sequence that originates from a foreign species, or, if from the same species, is substantially modified from its native form in composition and/or genomic locus by deliberate human intervention. A heterologous polynucleotide is also referred to herein as a “transgene”. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. While it may be optimal to express the sequences using heterologous promoters, the native promoter sequences may be used.

The termination region may be native with the transcriptional initiation region, may be native with the operably linked coding polynucleotide, may be native with the host cell, or may be derived from another source (i.e., foreign or heterologous) to the promoter, the coding polynucleotide, the host cell, or any combination thereof. Convenient termination regions are available from the potato proteinase inhibitor (PinII) gene or the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639. In some embodiments, the termination sequence that is operably linked to at least one of the site-specific recombinase-encoding polynucleotide, the selectable marker-encoding polynucleotide, the cell proliferation marker-encoding polynucleotide, the herbicide tolerance polynucleotide, and the polynucleotide of interest is the termination region from the pinII gene. In some of these embodiments, the termination region has the sequence set forth in SEQ ID NO: 1 or an active variant or fragment thereof that is capable of terminating transcription and/or translation in a plant cell.

The expression cassettes may additionally contain 5′ leader sequences. Such leader sequences can act to enhance translation. Translation leaders are known in the art and include: picornavirus leaders, for example, EMCV leader (encephalomyocarditis 5′ noncoding region) (Elroy-Stein et al. (1989) Proc. Natl. Acad. Sci. USA 86:6126-6130); potyvirus leaders, for example, TEV leader (tobacco etch virus) (Gallie et al. (1995) Gene 165(2):233-238), MDMV leader (maize dwarf mosaic virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

For example, in some of the embodiments, wherein the herbicide tolerance polynucleotide is a GLYAT polynucleotide, the cauliflower mosaic virus (CaMV) 35S enhancer region or tobacco mosaic virus (TMV) omega 5′ UTR translational enhancer element is included upstream of a promoter that is operably linked (when the excision cassette is excised) to the GLYAT polynucleotide to enhance transcription (see, for example, U.S. Pat. No. 7,928,296 and U.S. Pat. No. 7,622,641, each of which is herein incorporated by reference in its entirety).

In preparing the expression cassette or polynucleotide construct, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Expression cassettes comprise a promoter operably linked to a coding polynucleotide. As used herein, the term “promoter” includes reference to a region of DNA involved in the recognition and binding of RNA polymerase and other proteins to initiate transcription of a coding sequence. Promoters may be naturally occurring promoters, a variant or fragment thereof, or synthetically derived. The term “promoter” refers to the minimal sequences necessary to direct transcription (minimal promoter) as well as sequences comprising the minimal promoter and any number of additional elements, such as operator sequences, enhances, modulators, restriction sites, recombination sites, sequences located in between the minimal promoter and the coding sequence, and sequences of the 5′-untranslated region (5′-UTR), which is the region of a transcript that is transcribed, but is not translated into a polypeptide, which may or may not influence transcription levels in a desired manner. A “plant promoter” refers to a promoter isolated from a plant or a promoter derived therefrom or a heterologous promoter that functions in a plant.

Although according to the invention, the promoter that drives the expression of the site-specific recombinase is an inducible promoter, various types of promoters can be used for the regulation of the expression of the remaining coding polynucleotides in the presently disclosed polynucleotide constructs. The promoter may be selected based on the desired outcome or expression pattern (for a review of plant promoters, see Potenza et al. (2004) In Vitro Cell Dev Biol 40:1-22).

Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), the Agrobacterium nopaline synthase (NOS) promoter (Bevan et al. (1983) Nucl. Acids Res. 11:369-385); Mirabilis mosaic virus (MMV) promoter (Dey & Maiti (1999) Plant Mol Biol 40:771-782; Dey & Maiti (1999) Transgenics 3:61-70); histone 2B (H2B) (International Application Publication No. WO 99/43797); banana streak virus (BSV) promoter (Remans et al. (2005) Virus Research 108:177-186); chloris striate mosaic virus (CSMV) promoter (Zhan et al. (1993) Virology 193:498-502); Cassaya vein mosaic virus (CSVMV) promoter (Verdaguer et al. (1998) Plant Mol Biol 37:1055-1067); figwort mosaic virus (FMV) promoter (U.S. Pat. No. 6,018,100), rice alpha-tubulin (OsTUBA1) promoter (Jeon et al. (2000) Plant Physiol 123:1005-1014); rice cytochrome C (OsCC1) promoter (Jang et al. (2002) Plant Physiol 129:1473-1481); maize alcohol dehydrogenasel (ZmADH1) promoter (Kyozuka et al. (1990) Maydica 35:353-357; an oleosin promoter (e.g., SEQ ID NO: 2 or a variant or fragment thereof) and the like; each of which is herein incorporated by reference in its entirety. Other constitutive promoters are described in, for example, U.S. Pat. Nos. 5,608,149; 5,608,144; 5,604,121; 5,569,597; 5,466,785; 5,399,680; 5,268,463; 5,608,142; and U.S. Pat. No. 6,177,611; each of which is herein incorporated by reference in its entirety.

In some embodiments, an inducible promoter can be used, such as from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference. Promoters that are expressed locally at or near the site of pathogen infection include, for example, Marineau et al. (1987) Plant Mol. Biol. 9:335-342; Matton et al. (1989) Mol Plant-Microbe Interact 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1 :961-968; U.S. Pat. No. 5,750,386 (nematode-inducible); and the references cited therein.

Additional promoters include the inducible promoter for the maize PRms gene, whose expression is induced by the pathogen Fusarium moniliforme (see, for example, Cordero et al. (1992) Physiol. Mol. Plant. Path. 41:189-200). Wound-inducible promoters include potato proteinase inhibitor (pin II) gene (Ryan (1990) Ann. Rev. Phytopath. 28:425-449; Duan et al. (1996) Nat Biotechnol 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Lett 323:73-76); MPI gene (Corderok et al. (1994) Plant J. 6:141-150); and the like, herein incorporated by reference.

Other inducible promoters useful for regulating the expression of any of the coding sequences of the presently disclosed polynucleotide constructs include stress-inducible promoters, such as those described elsewhere herein.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners (De Veylder et al. (1997) Plant Cell Physiol. 38:568-77), the maize GST promoter (GST-II-27, WO 93/01294), which is activated by hydrophobic electrophilic compounds that are used as pre-emergent herbicides, the PR-1 promoter (Cao et al. (2006) Plant Cell Reports 6:554-60), which is activated by BTH or benxo(1,2,3)thiaidazole-7-carbothioic acid s-methyl ester, the tobacco PR-1a promoter (Ono et al. (2004) Biosci. Biotechnol. Biochem. 68:803-7), which is activated by salicylic acid, the copper inducible ACEI promoter (Mett et al. (1993) PNAS 90:4567-4571), the ethanol-inducible promoter AlcA (Caddick et al. (1988) Nature Biotechnol 16:177-80), an estradiol-inducible promoter (Bruce et al. (2000) Plant Cell 12:65-79), the XVE estradiol-inducible promoter (Zao et al. (2000) Plant J24:265-273), the VGE methoxyfenozide inducible promoter (Padidam et al. (2003) Transgenic Res 12:101-109), and the TGV dexamethasone-inducible promoter (Bohner et al. (1999) Plant J 19:87-95). Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237; Gatz et al. (1992) Plant J2:397-404; and U.S. Pat. No. 5,814,618 and U.S. Pat. No. 5,789,156), herein incorporated by reference.

One particular chemical-inducible promoter that is described in more detail elsewhere herein and that can be used in the presently disclosed compositions and methods, particularly to regulate the expression of the site-specific recombinase, is a promoter responsive to sulfonylurea, wherein the promoter comprises operator sequences capable of binding to a sulfonylurea-responsive transcriptional repressor (SuR) protein, such as those described in U.S. Application Publication Nos. 2010/0105141 and 2011/0287936, each of which is herein incorporated by reference in its entirety.

Tissue-preferred promoters can be utilized to target enhanced expression of a coding polynucleotide within a particular plant tissue. Tissue-preferred promoters include Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Lam (1994) Results Probl. Cell Differ. 20:181-196; and Guevara-Garcia et al. (1993) Plant J. 4(3):495-505.

Leaf-preferred promoters are known in the art. See, for example, Yamamoto et al. (1997) Plant J. 12:255-265; Kwon et al. (1994) Plant Physiol. 105:357-67; Yamamoto et al. (1994) Plant Cell Physiol. 35:773-778; Gotor et al. (1993) Plant J. 3:509-18; Orozco et al. (1993) Plant Mol. Biol. 23:1129-1138; and Matsuoka et al. (1993) Proc. Natl. Acad. Sci. USA 90:9586-9590. In addition, promoter of cab and rubisco can also be used. See, for example, Simpson et al. (1958) EMBO J 4:2723-2729 and Timko et al. (1988) Nature 318:57-58.

Root-preferred promoters are known and can be selected from the many available. See, for example, Hire et al. (1992) Plant Mol. Biol. 20:207-218 (soybean root-specific glutamine synthase gene); Keller and Baumgartner (1991) Plant Cell 3:1051-1061 (root-specific control element in the GRP 1.8 gene of French bean); Sanger et al. (1990) Plant Mol. Biol. 14:433-443 (root-specific promoter of the mannopine synthase (MAS) gene of Agrobacterium tumefaciens); and Miao et al. (1991) Plant Cell 3:11-22 (full-length cDNA clone encoding cytosolic glutamine synthase (GS), which is expressed in roots and root nodules of soybean). See also Bogusz et al. (1990) Plant Cell 2:633-641, where two root-specific promoters isolated from hemoglobin genes from the nitrogen-fixing nonlegume Parasponia andersonii and the related non-nitrogen-fixing nonlegume Trema tomentosa are described. Leach and Aoyagi (1991) describe their analysis of the promoters of the highly expressed rolC and rolD root-inducing genes of Agrobacterium rhizogenes (see Plant Sci (Limerick) 79:69-76). Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue (see EMBO J. 8:343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29:759-772); and rolB promoter (Capana et al. (1994) Plant Mol. Biol. 25:681-691. See also U.S. Pat. Nos. 5,837,876; 5,750,386; 5,633,363; 5,459,252; 5,401,836; 5,110,732; and U.S. Pat. No. 5,023,179. Another root-preferred promoter includes the promoter of the phaseolin gene (Murai et al. (1983) Science 23:476-482 and Sengopta-Gopalen et al. (1988) Proc. Natl. Acad. Sci. USA 82:3320-3324.

Seed-preferred promoters include both those promoters active during seed development as well as promoters active during seed germination. See Thompson et al. (1989) BioEssays 10:108, herein incorporated by reference. Such seed-preferred promoters include, but are not limited to, Cim1 (cytokinin-induced message); cZ19B1 (maize 19 kDa zein); and milps (myo-inositol-1-phosphate synthase); (see WO 00/11177 and U.S. Pat. No. 6,225,529; herein incorporated by reference). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, nuc1, etc. See also WO 00/12733, where seed-preferred promoters from end1 and end2 genes are disclosed; herein incorporated by reference.

Where low-level expression is desired, weak promoters will be used. Generally, by “weak promoter” is intended a promoter that drives expression of a coding sequence at a low level. By low level is intended at levels of about 1/1000 transcripts to about 1/100,000 transcripts to about 1/500,000 transcripts. Alternatively, it is recognized that weak promoters also encompasses promoters that are expressed in only a few cells and not in others to give a total low level of expression. Where a promoter is expressed at unacceptably high levels, portions of the promoter sequence can be deleted or modified to decrease expression levels. Such weak constitutive promoters include, for example, the core promoter of the Rsyn7 promoter (WO 99/43838 and U.S. Pat. No. 6,072,050), the core 35S CaMV promoter, and the like.

In some embodiments, at least one of the following promoters is a constitutive promoter: the promoter regulating the expression of the herbicide tolerance polypeptide, the promoter operably linked to the cell proliferation marker, and the promoter driving the expression of the selectable marker present within the excision cassette. In particular embodiments, the selectable marker present within the excision cassette of the presently disclosed polynucleotide constructs is operably linked to a constitutive promoter such that the selectable marker is constitutively expressed until excision of the excision cassette, and the same constitutive promoter then regulates the expression of the herbicide tolerance polypeptide upon excision of the cassette. In some of these embodiments, the constitutive promoter is the maize ubiquitin promoter (Christensen et al. (1989) Plant Mol. Biol. 12:619-632 and Christensen et al. (1992) Plant Mol. Biol. 18:675-689), which in some embodiments comprises the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113). In other embodiments, the constitutive promoter regulating the expression of the selectable marker present within the excision cassette is the enhanced Mirabilis mosaic virus (MMV) promoter (Dey & Maiti (1999) Plant Mol Biol 40:771-782; Dey & Maiti (1999) Transgenics 3:61-70). In some embodiments, the polynucleotide encoding a cell proliferation factor (e.g., babyboom polypeptide) is operably linked to a maize ubiquitin promoter (which in some embodiments comprises the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113) or a maize oleosin promoter (e.g., SEQ ID NO: 2 or a variant or fragment thereof).

According to the invention, the promoter that regulates the expression of the site-specific recombinase is an inducible promoter. In some embodiments, the inducible promoter that is operably linked to the site-specific recombinase-encoding polynucleotide comprises a stress-inducible promoter. As used herein, a “stress-inducible promoter” refers to a promoter that initiates transcription when the host cell (e.g., plant cell) or host (e.g., plant or plant part) undergoes stress, including abiotic stress. Non-limiting examples of conditions that can activate stress-inducible promoters include drought, salinity, flood, and suboptimal temperature. Some stress-inducible promoters are only activated by a particular stress (e.g., drought), whereas other stress-inducible promoters can be activated by any type of stress, particularly any type of abiotic stress.

Stress-inducible promoters include those that become activated in response to drought and high salinity (drought-inducible promoters) and cold temperatures (cold-inducible promoters). Some promoters are both drought-inducible and cold-inducible. Many stress-inducible promoters are also activated by abscisic acid (ABA), a phytohormone that is often expressed by plants in response to drought and high-salinity stress. Regulatory pathways by which stress-inducible promoters can become activated include those that are ABA-dependent as well as those that are ABA-independent. Thus, some stress-inducible promoters comprise an ABA-responsive element (ABRE) and respond to ABA. Some of those stress-inducible promoters that are responsive to drought, high salinity, and/or cold temperatures comprise a dehydration-responsive (DRE)/C-repeat (CRT) element. The C-repeat binding factor (CBF)/DREB1 transcription factor, the expression of which is induced by cold stress, and the DREB2 transcription factor, which is induced by dehydration, bind to DRE/CRT elements. In some embodiments, stress-inducible promoters comprise any one of the following cis-acting stress-responsive elements: ABRE, CE1, CE3, MYB recognition site (MYBR), MYC recognition site (MYCR), DRE, CRT, low-temperature-responsive element (LTRE), NAC recognition site (NACR), zinc-finger homeodomain recognition site (ZFHDR) and an inducer of CBF expression (ICE) recognition site. Table 1 provides the sequences of these cis-acting stress-responsive elements. See Yamaguchi-Shinozaki and Shinozaki (2005) Trends Plant Sci 10:1360-1385 and Shinozaki et al. (2003) Curr Opin Plant Biol 6:410-417, each of which is incorporated by reference in its entirety, for reviews of stress-inducible promoters and the regulatory pathways controlling the same.

TABLE 1
cis-Acting regulatory elements
in stress-inducible gene expression.*

| | | Type of | | |
|---|---|---|---|---|
| | | tran- | | |
| | | scrip- | | |
| | | tion- | | |
| | | factors | | |
| | | that | | |
| | | bind to | | |
| cis | Sequence | cis | | Stress |
| element | (SEQ ID NO:) | elements | Gene | condition |
| ABRE | PyACGTGGC (3) | bZIP | Em, | Water |
| | | | RAB16 | deficit, |
| | | | | ABA |
| CE1 | TGCCACCGG (4) | ERF/AP2 | HVA1 | ABA |
| CE3 | ACGCGTGCCTC | Not | HVA22 | ABA |
| | (5) | known | | |
| ABRE | ACGTGTC (6) | bZIP | Osem | ABA |
| ABRE | ACGTGGC (7), | bZIP | RD29B | Water |
| | ACGTGTC (8) | | | deficit, |
| | | | | ABA |
| MYBR | TGGTTAG (9) | MYB | RD22 | Water |
| | | | | deficit, |
| | | | | ABA |
| MYCR | CACATG (10) | bHLH | RD22 | Water |
| | | | | deficit, |
| | | | | ABA |
| DRE | TACCGACAT (11) | ERF/AP2 | RD29A | Water |
| | | | | deficit, |
| | | | | cold |
| CRT | GGCCGACAT (12) | ERF/AP2 | Cor15A | Cold |
| LTRE | GGCCGACGT (13) | ERF/AP2 | BN115 | Cold |
| NACR | ACACGCATGT | NAC | ERD1 | Water |
| | (14) | | | deficit |
| ZFHDR | Not yet | ZFHD | ERD1 | Water |
| | reported | | | deficit |
| ICEr1 | GGACACATGTCAGA | Not | CBF2/ | Cold |
| | (15) | known | DREB1C | |
| ICEr2 | ACTCCG (16) | Not | CBF2/ | Cold |
| | | known | DREB1C | |

*Adopted from Yamaguchi-Shinozaki and Shinozaki (2005) Trends Plant Sci 10:1360-1385

In some embodiments, the inducible promoter that is operably linked to the polynucleotide encoding a site-specific recombinase is a cold-inducible promoter. As used herein, a “cold-inducible promoter” is a promoter that is activated at temperatures that are below optimal temperatures for plant growth. In some embodiments, the cold-inducible promoter is one that is induced in response to temperatures less than about 20° C., less than about 19° C., less than about 18° C., less than about 17° C., less than about 16° C., less than about 15° C., less than about 14° C., less than about 13° C., less than about 12° C., less than about 11° C., less than about 10° C., less than about 9° C., less than about 8° C., less than about 7° C., less than about 6° C., less than about 5° C., less than about 4° C., less than about 3° C., less than about 2° C., less than about 1° C., or less than about 0° C.

Cold-inducible promoters may be activated by exposing a plant or plant part to cold temperatures for a period of about 12 hours, about 1 day, about 2 days, about 3 days, about 4 days, about 5 days, about 6 days, about 1 week, about 2 weeks, about 3 weeks, about 4 weeks, about 5 weeks, about 6 weeks, about 8 weeks, about 9 weeks, about 10 weeks, about 3 months, or more. The temperature required or the necessary amount of time the plant or plant part is exposed to the cold temperatures will vary based on, for example, the promoter, the plant species, the tyre of explant, and the size of the plant tissue, and can be determined by one of skill in the art.

Cold-inducible promoters can comprise a C-repeat (CRT) and/or a low-temperature-responsive element (LTRE), both of which contain an A/GCCGAC motif that forms the core of the DRE sequence, as well. Non-limiting examples of cold-inducible promoters include the maize rab17 promoter (Vilardell et al. (1990) Plant Mol Biol 14:423-432), the RD29A promoter (Uno et al. (2000) PNAS 97:11632-11637), the Cor15A promoter (Baker et al. (1994) Plant Mol Biol 24:701-713), the BN115 promoter (Jiang et al. (1996) Plant Mol Biol 30:679-684), and the CBF2/DREB1C promoter (Zarka et al. (2003) Plant Physiol 133:910-918); each of which is herein incorporated by reference in its entirety.

In some embodiments, the inducible promoter that regulates the expression of the site-specific recombinase is a vernalization promoter, which is a promoter that responds to cold exposure to trigger flowering in plants. Vernalization promoters generally require exposure to cold temperatures for an extended period of time (e.g., at least 2 weeks) for activation. In certain embodiments, activation of a vernalization promoter requires exposure to temperatures less than about 20° C., less than about 19° C., less than about 18° C., less than about 17° C., less than about 16° C., less than about 15° C., less than about 14° C., less than about 13° C., less than about 12° C., less than about 11° C., less than about 10° C., less than about 9° C., less than about 8° C., less than about 7° C., less than about 6° C., less than about 5° C., less than about 4° C., less than about 3° C., less than about 2° C., less than about 1° C., or less than about 0° C. for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, or more. In certain embodiments, activation of a vernalization promoter requires exposure to a temperature of about 4° C. for about 2 weeks.

In some embodiments, the vernalization promoter comprises a putative MADS-box protein binding site, referred to herein as CarG-box, the sequence of which is set forth in SEQ ID NO: 114. A non-limiting example of a vernalization promoter is the Triticum monococcum VRN1/AP1 promoter set forth in SEQ ID NO: 115 and described in Yan et al. (2003) Proc Natl Acad Sci USA 100:6263-6268 and U.S. Application Publication No. 2004/0203141, each of which is herein incorporated by reference in its entirety.

In some of those embodiments wherein the inducible promoter that regulates the expression of the site-specific recombinase is a vernalization promoter, the host cell of the polynucleotide construct is a Brassica sp., winter wheat, barley, oat, or rye.

In other embodiments, the inducible promoter that regulates the expression of the site-specific recombinase is a drought-inducible promoter. As used herein, a “drought-inducible promoter” or “desiccation-inducible promoter” refers to a promoter that initiates transcription in response to drought conditions, high salinity, and/or dessication of a plant or plant part. Drought-inducible promoters can drive expression in a number of different plant tissues including, but not limited to, root tissue (e.g., root endodermis, root epidermis, or root vascular tissues) and leaf tissue (e.g. epidermis, mesophyll or leaf vascular tissue).

In some embodiments, the drought-inducible promoter comprises a DRE or an early responsive to dehydration 1 (ERD1) cis-acting element (Yamaguchi-Shinozaki and Shinozaki (2004) Trends Plant Sci 10:1360-1385; and Shinozaki et al. (2003) Curr Opin Plant Biol 6:410-417).

The drought-inducible promoter is activated when the plant or plant part comprising the same is desiccated. As used herein, the term “desiccate” refers to a process by which the water content of a plant or plant part is reduced, and can include reference to the natural desiccation process that occurs during the maturation of seeds. Thus, in some embodiments, the drought-inducible promoter is activated in a plant cell comprising the presently disclosed polynucleotide constructs and excision of the excision cassette occurs during the maturation of a seed comprising the plant cell.

A desiccated plant or plant part can comprise about 90%, 85%, 80%, 75%, 70%, 65%, 60%, 55%, 50%, 45%, 40%, 35%, 30%, 25%, 20%, 15%, 10%, 5%, 1%, 0.1% or less water than a plant or plant part that has not been dried. The amount of desiccation necessary to activate a drought-inducible promoter or the amount of time needed to desiccate a plant or plant part will vary based on, for example, the promoter, the plant species, the explant type, and the size of the plant tissue.

In some embodiments, a plant or plant part is desiccated and the drought-inducible promoter is activated by exposing the plant or plant part comprising the drought-inducible promoter to drought conditions. As used herein, “drought” or “drought conditions” can be defined as the set of environmental conditions under which a plant or plant part will begin to suffer the effects of water deprivation, such as decreased stomatal conductance and photosynthesis, decreased growth rate, loss of turgor (wilting), or ovule abortion. For these reasons, plants experiencing drought stress typically exhibit a significant reduction in biomass and yield. Water deprivation may be caused by lack of rainfall or limited irrigation. Alternatively, water deficit may also be caused by high temperatures, low humidity, saline soils, freezing temperatures or water-logged soils that damage roots and limit water uptake to the shoot. Since plant species vary in their capacity to tolerate water deficit, the precise environmental conditions that cause drought stress cannot be generalized.

The drought-inducible promoter may be activated by exposing a plant or plant part to drought conditions for a period of about 1 day, about 2 days, about 3 days, about 4 days, about 5 days, about 6 days, about 1 week, about 2 weeks, about 3 weeks, or more.

In some embodiments, the plant or plant part is desiccated and the drought-inducible promoter activated by incubating the plant or plant part in the absence of liquid medium and optionally on dry filter paper. In some embodiments, the plant or plant part is desiccated by incubating the plant or plant part in a sealed container with a saturated salt solution (e.g., (NH₄)₂SO₄). In some embodiments, the plant or plant part is incubated in the absence of liquid medium, and optionally, on dry filter paper, and in some embodiments, in a sealed container with a saturated salt solution for about 1 day, about 1.5 days, about 2 days, about 2.5 days, about 3 days, about 3.5 days, about 4 days, about 4.5 days, about 5 days, about 5.5 days, about 6 days, about 6.5 days, about 7 days, about 7.5 days, about 8 days, about 8.5 days, about 9 days, about 9.5 days, about 10 days, or more in order to induce the expression of the drought-inducible promoter.

Non-limiting examples of drought-inducible promoters include the promoters of maize rab17 (Vilardell et al. (1990) Plant Mol Biol 14:423-432): Oryza saliva Em (Guiltinan et al. (1990) Science 250:267-271); Rab16 (Mundy et al. (1990) PNAS 87:406-410); HVA1 (Hobo et al. (1999) Plant J 19:679-689); HVA22 (Su et al. (1998) Plant Physiol 117:913-922); RD29B and RD29A (Uno et al. (2000) PNAS 97:11632-11637); RD22 (Abe et al (1997) Plant Cell 9:1859-1868); Cor15A (Baker et al. (1994) Plant Mol Biol 24:701-713); BN115 (Jiang et al. (1996) Plant Mol Biol 30:679-684); ERD1 (Tran et al. (2004) Plant Cell 16:2481-2498); Oryza sativa LEA3 (Xiao et al. (2007) Theor Appl Genet 115:35-46); Oryza sativa rab16Bj (Xiao and Xue (2001) Plant Cell Rep 20:667-73); Brassica LEA3-1 (U.S. Application Publication No. US 2008/0244793); LEA D7, LEA D11, LEA D19, LEA d34, and LEA D113 (Baker et al. (1988) Plant Mol Biol 11:277-291); Oryza sativa RAB16 and Sorghum bicolor DHN2 (Buchanan et al. (2004) Genetics 168:1639-1654); Oryza sativa ASR1 (Kuriakose et al. (2009) African J Biotech 8:4765-73); Oryza sativa NAC6 (Nakashima et al. (2007) Plant J 51:617-630); Oryza sativa SALT (Garcia et al. (1998) Planta 207:172-180); Oryza sativa LIPS (Aguan et al. (1993) Mol Gen Genet 240:1-8); Oryza sativa WS1724 (Takahashi et al. (1994) Plant Mol Biol 26:339-352); Oryza sativa WSI18 (Oh et al. (2005) Plant Physiol 138:341-351); AREB1, AREB2, and ABF3 (Yoshida et al. (2010) Plant J 61:672-685); Oryza sativa DIP1, UGE1, R1G1B, and RAB21 promoters (Yi et al. (2010) Planta 232:743-754); cotton D113 (Luo et al. (2008) Plant Cell Rep 27:707-717); the dehydrin promoter; the ASI promoter; the WGA promoter; the P511 promoter; and the HS70 promoter; the dehydrin (DHN) promoter (Robertson et al. (1995) Physiol Plant 94:470-478); the alpha-amylase/subtilisin inhibitor (ASI) promoter (Furtado et al. (2003) Plant Mol Biol 52:787-799); the WGA promoter; and the HS70 promoter; each of which is herein incorporated by reference in its entirety.

In some embodiments, the inducible promoter that drives the expression of a site-specific recombinase and subsequent excision of the excision cassette is a Rab17 promoter, such as the maize rab17 promoter or an active variant or fragment thereof. The maize rab17 (responsive to abscisic acid) gene (GenBank Accession No. X15994; Vilardell et al. (1990) Plant Mol Biol 14:423-432; Vilardell et al. (1991) Plant Mol Biol 17:985-993; each of which is herein incorporated in its entirety) is expressed in late embryos, but its expression can be induced by exposure to abscisic acid, cold temperatures, or water stress. The sequence of the maize rab17 promoter corresponds to nucleotides 1-558 of GenBank Accession No. X15994, which was disclosed in Vilardell et al. (1990) Plant Mol Biol 14:423-432 and is set forth in SEQ ID NO: 17. An alternative maize rab17 promoter was disclosed in U.S. Pat. Nos. 7,253,000 and 7,491,813, each of which is herein incorporated by reference in its entirety, and is set forth in SEQ ID NO: 18. The rab17 promoter contains four abscisic acid responsive elements (ABRE) (Busk et al. (1997) Plant J 11:1285-1295, which is herein incorporated by reference in its entirety). The ABRE elements in the maize rab17 promoter can be found at nucleotides 304-309, 348-353, 363-368, 369-374, 414-419, and 427-432 of SEQ ID NO: 18. The rab17 promoter also contains drought-responsive elements (DRE), of which the core sequence is identical to the DRE (drought-responsive) and CRT (cold-response elements) elements in Arabidopsis. The drought-responsive elements of the maize rab17 promoter are found at nucleotides 233-238, 299-304, and 322-327 of SEQ ID NO: 18. The CAAT and TATAA box can be found from nucleotides 395 to 398 and 479 to 483 of SEQ ID NO: 18, respectively. In those embodiments wherein the inducible promoter that regulates the expression of the site-specific recombinase is a rab17 promoter, the expression of the recombinase can be induced by desiccating a host cell (e.g., plant cell) or host (e.g., plant or plant part) or exposing the host cell or host to drought conditions, cold temperatures, or abscisic acid.

In some embodiments, the stress-inducible promoter of the presently disclosed polynucleotide constructs has the sequence set forth in SEQ ID NO: 18 or an active variant or fragment thereof. In other embodiments, the stress-inducible promoter of the presently disclosed polynucleotide constructs has the sequence set forth in SEQ ID NO: 17 or 19 or an active variant or fragment thereof.

In some embodiments of the methods and compositions, the polynucleotide constructs comprise active variants or fragments of the maize rab17 promoter. An active variant or fragment of a maize rab17 promoter (e.g., SEQ ID NO: 17, 18, 19) is a polynucleotide variant or fragment that retains the ability to initiate transcription in response to drought conditions, desiccation, cold, and/or ABA. In some of these embodiments, the promoter comprises at least one DRE element. In some embodiments, an active fragment of a maize rab17 promoter may comprise at least about 50, 100, 150, 200, 250, 300, 350, 400, 450, or 500 contiguous nucleotides of SEQ ID NO: 17, 18, or 19, or may have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to SEQ ID NO: 17, 18, or 19. In particular embodiments, the promoter of the compositions and methods comprises from about −219 to about −102 of the maize rab17 promoter (corresponding to nucleotides 291 to 408 of SEQ ID NO: 18). In other embodiments, the active maize rab17 promoter fragment comprises from about −219 to about −80 of the maize rab17 promoter (nucleotides 291 to 430 of SEQ ID NO: 18), which comprises most of the DRE and ABRE elements.

In some embodiments, the expression of the site-specific recombinase is regulated by a promoter comprising a maize rab17 promoter or a fragment or variant thereof, and an attachment site, such as an attachment B (attB) site as described in U.S. Application Publication No. 2011/0167516 (which is herein incorporated by reference in its entirety), and in some of these embodiments, the attB site modifies the activity of the maize rab17 promoter.

As used herein, a “modulator” refers to a polynucleotide that when present between a promoter and a coding sequence, serves to increase or decrease the activity of the promoter. Non-limiting examples of modulators include recombination sites, operators, and insulators.

Attachment sites are site-specific recombination sites found in viral and bacterial genomes that facilitate the integration or excision of the viral genome into and out of its host genome. Non-limiting examples of a viral and bacterial host system that utilize attachment sites is the lambda bacteriophage and E. coli system (Weisberg and Landy (1983) In Lambda II, eds. Hendrix et al. (Cold Spring Harbor Laboratory, Cold Spring Harbor, N.Y.) pp. 211-250). The modulator of the maize rab17 promoter can be an E. coli attachment site B (attB) site. The attB site can be a naturally occurring E. coli attB site or an active variant or fragment thereof or a synthetically derived sequence. Synthetically derived attB sites and active variants and fragments of naturally occurring attB sites are those that are capable of recombining with a bacteriophage lambda attachment P site, a process that is catalyzed by the bacteriophage lambda Integrase (Int) and the E. coli Integration Host Factor (IHF) proteins (Landy (1989) Ann Rev Biochem 58: 913-949, which is herein incorporated by reference in its entirety). AttB sites typically have a length of about 25 nucleotides, with a core 15-base pair sequence that is involved in the actual crossover event. Alternatively, active variants and fragments of naturally occurring attB sites are those that are capable of modulating the activity of a promoter. Non-limiting examples of attB sites that can be used include attB1 (SEQ ID NO: 20), attB2 (SEQ ID NO: 21), attB3 (SEQ ID NO: 22), and attB4 (SEQ ID NO: 23), and variants or fragments thereof. In some embodiments, the modulator is an active variant or fragment of an attB site that is capable of modulating (i.e., increasing, decreasing) the activity of a promoter, but is not capable of recombination with an attachment P site. Non-limiting examples of such active variants of an attB site include those having the sequence set forth in SEQ ID NO: 24, 25, or 26.

In some embodiments, the distance of the modulator (e.g., attB site) from the promoter impacts the ability of the modulator to modify the activity of the promoter. The modulator may be contiguous with the promoter and/or the coding polynucleotide. In other embodiments, a linker sequence separates the promoter sequence and the modulator (e.g., attB site). As used herein, a “linker sequence” is a nucleotide sequence that functions to link one functional sequence with another without otherwise contributing to the expression or translation of a coding polynucleotide. Accordingly, the actual sequence of the linker sequence can vary. The linker sequence can comprise plasmid sequences, restriction sites, and/or regions of the 5′-untranslated region (5′-UTR) of the gene from which the promoter is derived. The linker sequence separating the promoter and the modulator (e.g., attB site) can have a length of about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 1000 nucleotides or greater. In certain embodiments, a linker sequence of about 133 nucleotides separates the maize rab17 promoter and the modulator (e.g., attB site). In some embodiments, the linker sequence comprises a fragment of the rab17 5′-UTR. The fragment of the 5′-UTR can be about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 nucleotides, or greater, in length. In certain embodiments, the promoter comprises a linker sequence separating the maize rab17 promoter and the modulator (e.g., attB site) that comprises 95 nucleotides of the maize rab17 5′-UTR. In some of these embodiments, the 95 nucleotide sequence has the sequence set forth in SEQ ID NO: 27. In certain embodiments, the linker sequence between the maize rab17 promoter and modulator (e.g., attB site) has the sequence set forth in SEQ ID NO: 28 or a variant or fragment thereof.

In some embodiments, the promoter comprises a linker sequence separating the modulator (e.g., attB site) and the site-specific recombinase-coding polynucleotide. The length and sequence of this linker may also vary and can be about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 1000 nucleotides or greater in length. In certain embodiments, a linker sequence of about 61 nucleotides separates the modulator (e.g., attB site) and the recombinase-encoding polynucleotide. In certain embodiments, the linker sequence between the modulator (e.g., attB site) and the coding polynucleotide has the sequence set forth in SEQ ID NO: 29 or a variant or fragment thereof. In other embodiments, a linker sequence of about 25 nucleotides separates the modulator (e.g., attB site) and the coding polynucleotide. In certain embodiments, the linker sequence between the modulator (e.g., attB site) and the coding polynucleotide has the sequence set forth in SEQ ID NO: 30.

In certain embodiments, the stress-inducible promoter that regulates the expression of the site-specific recombinase has the sequence set forth in SEQ ID NO: 31 or a variant or fragment thereof.

In other embodiments of the presently disclosed compositions and methods, the inducible promoter that regulates the expression of the site-specific recombinase is a chemical-inducible promoter. In some of these embodiments, the chemical-inducible promoter is a sulfonylurea (SU)-inducible promoter that has at least one operator sequence capable of binding to a sulfonylurea-responsive transcriptional repressor (SuR) protein, such as those disclosed in U.S. Application Publication Nos. 2010/0105141 and 2011/0287936.

As used herein, a “sulfonylurea-responsive transcriptional repressor” or “SuR” refers to a transcriptional repressor protein whose binding to an operator sequence is controlled by a ligand comprising a sulfonylurea compound. The SuR proteins useful in the presently disclosed methods and compositions include those that bind specifically to an operator sequence in the absence of a sulfonylurea ligand.

In some embodiments, the SuR protein is one that specifically binds to a tetracycline operator, wherein the specific binding is regulated by a sulfonylurea compound. Thus, in some embodiments, the sulfonylurea-inducible promoter comprises at least one tetracycline (tet) operator sequence. Tetracycline operator sequences are known in the art and include the tet operator sequence set forth in SEQ ID NO: 32. The tet operator sequence can be located within 0-30 nucleotides 5′ or 3′ of the TATA box of the chemical-regulated promoter, including, for example, within 20, 19, 18, 17, 16, 15, 14, 13, 12, 11, 10, 9, 8, 7, 6, 5, 4, 3, 2, 1, or 0 nt of the TATA box. In other instances, the tet operator sequence may partially overlap with the TATA box sequence. In one non-limiting example, the tet operator sequence is SEQ ID NO: 32 or an active variant or fragment thereof.

Useful tet operator containing promoters include, for example, those known in the art (see, e.g., Matzke et al. (2003) Plant Mol Biol Rep 21:9-19; Padidam (2003) Curr Op Plant Biol 6:169-177; Gatz & Quail (1988) PNAS 85:1394-1397; Ulmasov et al. (1997) Plant Mol Biol 35:417-424; Weinmann et al. (1994) Plant J 5:559-569; each of which is herein incorporated by reference in its entirety). One or more tet operator sequences can be added to a promoter in order to produce a sulfonylurea-inducible promoter. See, for example, Weinmann et al. (1994) Plant J 5:559-569; Love et al. (2000) Plant J 21:579-588. In addition, the widely tested tetracycline regulated expression system for plants using the CaMV 35S promoter (Gatz et al. (1992) Plant J2:397-404; which is herein incorporated by reference in its entirety) having three tet operators introduced near the TATA box (3XOpT 35S) can be used as the sulfonylurea-inducible promoter.

Thus, a SU-inducible promoter comprising at least one, two, three or more operators capable of binding a SuR (including a tet operator, such as that set forth in SEQ ID NO:32 or an active variant or fragment thereof) can be used to regulate the expression of the site-specific recombinase. Any promoter can be combined with an operator capable of binding a SuR to generate a SU-inducible promoter. In specific embodiments, the promoter is active in plant cells. The promoter can be a constitutive promoter or a non-constitutive promoter. Non-constitutive promoters include tissue-preferred promoter, such as a promoter that is primarily expressed in roots, leaves, stems, flowers, silks, anthers, pollen, meristem, seed, endosperm, or embryos.

In particular embodiments, the promoter is a plant actin promoter, a banana streak virus promoter (BSV), an MMV promoter, an enhanced MMV promoter (dMMV), a plant P450 promoter, or an elongation factor 1a (EF1A) promoter (U.S. Application Publication No. 20080313776, which is herein incorporated by reference in its entirety).

In those embodiments wherein the inducible promoter that is operably linked to the polynucleotide encoding the site-specific recombinase is a SU-inducible promoter, the host cell further comprises a sulfonylurea-responsive transcriptional repressor (SuR) or the polynucleotide construct comprises a polynucleotide encoding a SuR. Non-limiting examples of SuR polynucleotide and polypeptide sequences include those disclosed in U.S. Application Publication No. 2011/0287936, such as the polypeptide sequences set forth in SEQ ID NOs: 3-419 and the polynucleotide sequences set forth in SEQ ID NOs: 420-836 of U.S. Application Publication No. 2011/0287936, which is herein incorporated by reference in its entirety. Additional non-limiting examples of SuR polynucleotide and polypeptide sequences include those disclosed in U.S. Application Publication No. 2010/0105141, such as the polypeptide sequences set forth in SEQ ID NO: 3-401, 1206-1213, 1228-1233, and 1240-1243 and the polynucleotide sequences set forth in SEQ ID NO: 434-832, 1214-1221, 1222-1227, 1234-1239, and 1244-1247 of U.S. Application Publication No. 2010/0105141, which is herein incorporated by reference in its entirety.

In those embodiments wherein the presently disclosed polynucleotide constructs further comprise a polynucleotide encoding a SuR, the SuR-encoding polynucleotide is operably linked to a promoter that is active in a plant. The promoter may be a constitutive or a non-constitutive promoter, including a tissue-preferred promoter.

In particular embodiments, the promoter that is operably linked to the SuR-encoding polynucleotide comprises operator sequences that are capable of binding to SuR, which allows for autoregulation of the repressor and enhanced induction of the SU-inducible promoter and expression of the site-specific recombinase. See, for example, U.S. Application Publication No. 2011/0287936.

In particular embodiments, the SuR-encoding polynucleotide and optionally, the promoter operably linked thereto, is present within the excision cassette of the presently disclosed polynucleotide constructs, such that the polynucleotide is excised upon induction of the SU-inducible promoter and expression of the site-specific recombinase.

A variety of SU compounds can be used to bind to the SuR and induce the SU-inducible promoter. Sulfonylurea molecules comprise a sulfonylurea moiety (—S(O)₂NHC(O)NH(R)—). In sulfonylurea herbicides, the sulfonyl end of the sulfonylurea moiety is connected either directly or by way of an oxygen atom or an optionally substituted amino or methylene group to a typically substituted cyclic or acyclic group. At the opposite end of the sulfonylurea bridge, the amino group, which may have a substituent such as methyl (R being CH₃) instead of hydrogen, is connected to a heterocyclic group, typically a symmetric pyrimidine or triazine ring, having one or two substituents such as methyl, ethyl, trifluoromethyl, methoxy, ethoxy, methylamino, dimethylamino, ethylamino and the halogens. Sulfonylurea herbicides can be in the form of the free acid or a salt. In the free acid form, the sulfonamide nitrogen on the bridge is not deprotonated (i.e., —S(O)₂NHC(O)NH(R)), while in the salt form, the sulfonamide nitrogen atom on the bridge is deprotonated, and a cation is present, typically of an alkali metal or alkaline earth metal, most commonly sodium or potassium. Sulfonylurea compounds include, for example, compound classes such as pyrimidinylsulfonylurea compounds, triazinylsulfonylurea compounds, thiadiazolylurea compounds, and pharmaceuticals such as antidiabetic drugs, as well as salts and other derivatives thereof. Examples of pyrimidinylsulfonylurea compounds include amidosulfuron, azimsulfuron, bensulfuron, bensulfuron-methyl, chlorimuron, chlorimuron-ethyl, cyclosulfamuron, ethoxysulfuron, flazasulfuron, flucetosulfuron, flupyrsulfuron, flupyrsulfuron-methyl, foramsulfuron, halosulfuron, halosulfuron-methyl, imazosulfuron, mesosulfuron, mesosulfuron-methyl, nicosulfuron, orthosulfamuron, oxasulfuron, primisulfuron, primisulfuron-methyl, pyrazosulfuron, pyrazosulfuron-ethyl, rimsulfuron, sulfometuron, sulfometuron-methyl, sulfosulfuron, trifloxysulfuron and salts and derivatives thereof. Examples of triazinylsulfonylurea compounds include chlorsulfuron, cinosulfuron, ethametsulfuron, ethametsulfuron-methyl, iodosulfuron, iodosulfuron-methyl, metsulfuron, metsulfuron-methyl, prosulfuron, thifensulfuron, thifensulfuron-methyl, triasulfuron, tribenuron, tribenuron-methyl, triflusulfuron, triflusulfuron-methyl, tritosulfuron and salts and derivatives thereof. Examples of thiadiazolylurea compounds include buthiuron, ethidimuron, tebuthiuron, thiazafluoron, thidiazuron, pyrimidinylsulfonylurea compound (e.g., amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulftiron, pyrazosulfuron, rimsulfuron, sulfometuron, sulfosulfuron and trifloxysulfuron); a triazinylsulfonylurea compound (e.g., chlorsulfuron, cinosulfuron, ethametsulfuron, iodosulfuron, metsulfuron, prosulfuron, thifensulfuron, triasulfuron, tribenuron, triflusulfuron and tritosulfuron); or a thiadazolylurea compound (e.g., cloransulam, diclosulam, florasulam, flumetsulam, metosulam, and penoxsulam) and salts and derivatives thereof. Examples of antidiabetic drugs include acetohexamide, chlorpropamide, tolbutamide, tolazamide, glipizide, gliclazide, glibenclamide (glyburide), gliquidone, glimepiride and salts and derivatives thereof. In some systems, the SuR polypeptides specifically bind to more than one sulfonylurea compound, so one can chose which SU ligand to apply to the plant.

In some examples, the sulfonylurea compound is selected from the group consisting of chlorsulfuron, ethametsulfuron-methyl, metsulfuron-methyl, thifensulfuron-methyl, sulfometuron-methyl, tribenuron-methyl, chlorimuron-ethyl, nicosulfuron, and rimsulfuron.

In other embodiments, the sulfonylurea compound comprises a pyrimidinylsulfonylurea, a triazinylsulfonylurea, a thiadazolylurea, a chlorosulfuron, an ethametsulfuron, a thifensulfuron, a metsulfuron, a sulfometuron, a tribenuron, a chlorimuron, a nicosulfuron, or a rimsulfuron compound.

In some embodiments, it may be necessary for a plant or plant part that is contacted with a SU in order to induce the SU-inducible promoter to have tolerance to the SU. A host (e.g., a plant or plant part) may be naturally tolerant to the SU ligand, or the host (e.g., the plant or plant part) may be tolerant to the SU ligand as a result of human intervention such as, for example, by the use of a recombinant construct, plant breeding or genetic engineering. Thus, the host (e.g., the plant or plant part) employed in the various methods disclosed herein can comprise a native or a heterologous sequence that confers tolerance to the sulfonylurea compound.

In some of these embodiments, the presently disclosed polynucleotide constructs can comprise a polynucleotide encoding a sulfonylurea-tolerance polypeptide, which is a polypeptide that when expressed in a host (e.g., plant or plant part) confers tolerance to at least one sulfonylurea. In some of these embodiments, the polynucleotide encoding the SU-tolerance polypeptide is comprised within the excision cassette.

In other embodiments, the herbicide tolerance polypeptide that is expressed upon excision of the excision cassette is a SU-tolerance polypeptide, such that the plant or plant part does not have tolerance to SU prior to the addition of SU to the plant or plant part, but upon the addition of SU, the excision cassette is excised and the SU-tolerance polypeptide is subsequently expressed, which allows for protection of the plant or plant part from damage due to the SU.

Sulfonylurea herbicides inhibit growth of higher plants by blocking acetolactate synthase (ALS), also known as, acetohydroxy acid synthase (AHAS). Thus, in some embodiments, the SU-tolerance polypeptide is an ALS inhibitor-tolerance polypeptide, as described elsewhere herein.

When the inducible promoter of the presently disclosed polynucleotide constructs is activated, a site-specific recombinase is expressed, which catalyzes the excision of the excision cassette comprised within the polynucleotide construct. As used herein, an “excision cassette” refers to a polynucleotide that is flanked by recombination sites that are recombinogenic with one another and directly repeated, such that when acted upon by a site-specific recombinase that recognizes the recombination sites, the nucleotide sequence within the recombination sites is excised from the remaining polynucleotide. The excision cassette of the presently disclosed polynucleotide constructs comprise a first expression cassette comprising a site-specific recombinase-encoding polynucleotide operably linked to an inducible promoter and optionally, at least one of a polynucleotide encoding a selectable marker, a polynucleotide encoding a cell proliferation factor, a polynucleotide encoding a herbicide tolerance polypeptide, and a polynucleotide of interest.

A site-specific recombinase, also referred to herein as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. The recombinase used in the methods and compositions can be a native recombinase or a biologically active fragment or variant of the recombinase. For reviews of site-specific recombinases and their recognition sites, see Sauer (1994) Curr Op Biotechnol 5:521-527; and Sadowski (1993) FASEB 7:760-767, each of which is herein incorporated by reference in its entirety.

Any recombinase system can be used in the presently disclosed methods and compositions. Non-limiting examples of site-specific recombinases include FLP, Cre, S-CRE, V-CRE, Dre, SSV1, lambda Int, phi C31 Int, HK022, R, Gin, Tn1721, CinH, ParA, Tn5053, Bxb1, TP907-1, U153, and other site-specific recombinases known in the art, including those described in Thomson and Ow (2006) Genesis 44:465-476, which is herein incorporated by reference in its entirety. Examples of site-specific recombination systems used in plants can be found in U.S. Pat. Nos. 5,929,301, 6,175,056, 6,331,661; and International Application Publication Nos. WO 99/25821, WO 99/25855, WO 99/25841, and WO 99/25840, the contents of each are herein incorporated by reference.

In some embodiments, the recombinase is a member of the Integrase or Resolvase families, including biologically active variants and fragments thereof. The Integrase family of recombinases has over one hundred members and includes, for example, FLP, Cre, lambda integrase, and R. For other members of the Integrase family, see, for example, Esposito et al. (1997) Nucleic Acids Res 25:3605-3614; and Abremski et al. (1992) Protein Eng 5:87-91; each of which are herein incorporated by reference in its entirety. Other recombination systems include, for example, the Streptomycete bacteriophage phi C31 (Kuhstoss et al. (1991) J Mol Biol 20:897-908); the SSV1 site-specific recombination system from Sulfolobus shibatae (Maskhelishvili et al. (1993) Mol Gen Genet. 237:334-342); and a retroviral integrase-based integration system (Tanaka et al. (1998) Gene 17:67-76). In some embodiments, the recombinase does not require cofactors or a supercoiled substrate. Such recombinases include Cre, FLP, or active variants or fragments thereof.

The FLP recombinase is a protein that catalyzes a site-specific reaction that is involved in amplifying the copy number of the two-micron plasmid of S. cerevisiae during DNA replication. FLP recombinase catalyzes site-specific recombination between two FRT sites. The FLP protein has been cloned and expressed (Cox (1993) Proc Natl Acad Sci USA 80:4223-4227, which is herein incorporated by reference in its entirety). The FLP recombinase for use in the methods and compositions may be derived from the genus Saccharomyces. In some embodiments, a recombinase polynucleotide modified to comprise more plant-preferred codons is used. A recombinant FLP enzyme encoded by a nucleotide sequence comprising maize preferred codons (FLPm) that catalyzes site-specific recombination events is known (the polynucleotide and polypeptide sequence of which is set forth in SEQ ID NO: 33 and 34, respectively; see, e.g., U.S. Pat. No. 5,929,301, which is herein incorporated by reference in its entirety). Additional functional variants and fragments of FLP are known (Buchholz et al. (1998) Nat Biotechnol 16:657-662; Hartung et al. (1998) J Biol Chem 273:22884-22891; Saxena et al. (1997) Biochim Biophys Acta 1340:187-204; Hartley et al. (1980) Nature 286:860-864; Voziyanov et al. (2002) Nucleic Acids Res 30:1656-1663; Zhu & Sadowski (1995) J Biol Chem 270:23044-23054; and U.S. Pat. No. 7,238,854, each of which is herein incorporated by reference in its entirety).

The bacteriophage recombinase Cre catalyzes site-specific recombination between two lox sites. The Cre recombinase is known (Guo et al. (1997) Nature 389:40-46; Abremski et al. (1984) J Biol Chem 259:1509-1514; Chen et al. (1996) Somat Cell Mol Genet 22:477-488; Shaikh et al. (1977) J Biol Chem 272:5695-5702; and, Buchholz et al. (1998) Nat Biotechnol 16:657-662, each of which is herein incorporated by reference in its entirety). Cre polynucleotide sequences may also be synthesized using plant-preferred codons, for example such sequences (moCre; the polynucleotide and polypeptide sequence of which is set forth in SEQ ID NO: 35 and 36, respectively) are described, for example, in International Application Publication No. WO 99/25840, which is herein incorporated by reference in its entirety. Variants of the Cre recombinase are known (see, for example U.S. Pat. No. 6,890,726; Rufer & Sauer (2002) Nucleic Acids Res 30:2764-2772; Wierzbicki et al. (1987) J Mol Biol 195:785-794; Petyuk et al. (2004) J Biol Chem 279:37040-37048; Hartung & Kisters-Woike (1998) J Biol Chem 273:22884-22891; Santoro & Schultz (2002) Proc Natl Acad Sci USA 99:4185-4190; Koresawa et al. (2000) J Biochem (Tokyo) 127:367-372; and Vergunst et al. (2000) Science 290:979-982, each of which are herein incorporated by reference in its entirety).

In some embodiments, the recombinase is a S-CRE, V-CRE recombinase (Suzuki & Nakayama (2011) Nucl Acid Res 39(8):e49) or Dre recombinase (Sauer & McDermott (2004) Nucl Acid Res 32(20):6086-6095), each of which is herein incorporated by reference in its entirety.

In some embodiments, the recombinase is a chimeric recombinase, which is a recombinant fusion protein that is capable of catalyzing site-specific recombination between recombination sites that originate from different recombination systems. For example, if the set of recombination sites comprises a FRT site and a LoxP site, a chimeric FLP/Cre recombinase or active variant or fragment thereof can be used, or both recombinases may be separately provided. Methods for the production and use of such chimeric recombinases or active variants or fragments thereof are described, for example, in International Application Publication No. WO 99/25840; and Shaikh & Sadowski (2000) J Mol Biol 302:27-48, each of which are herein incorporated by reference in its entirety.

In other embodiments, a variant recombinase is used. Methods for modifying the kinetics, cofactor interaction and requirements, expression, optimal conditions, and/or recognition site specificity, and screening for activity of recombinases and variants are known, see for example Miller et al. (1980) Cell 20:721-9; Lange-Gustafson and Nash (1984) J Biol Chem 259:12724-32; Christ et al. (1998) J Mol Biol 288:825-36; Lorbach et al. (2000) J Mol Biol 296:1175-81; Vergunst et al. (2000) Science 290:979-82; Dorgai et al. (1995) J Mol Biol 252:178-88; Dorgai et al. (1998) J Mol Biol 277:1059-70; Yagu et al. (1995) J Mol Biol 252:163-7; Sclimente et al. (2001) Nucleic Acids Res 29:5044-51; Santoro and Schultze (2002) Proc Natl Acad Sci USA 99:4185-90; Buchholz and Stewart (2001) Nat Biotechnol 19:1047-52; Voziyanov et al. (2002) Nucleic Acids Res 30:1656-63; Voziyanov et al. (2003) J Mol Biol 326:65-76; Klippel et al. (1988) EMBO J. 7:3983-9; Arnold et al. (1999) EMBO J 18:1407-14; and International Application Publication Nos. WO 03/08045, WO 99/25840, and WO 99/25841; each of which is herein incorporated by reference in its entirety.

By “recombination site” is intended a polynucleotide (native or synthetic/artificial) that is recognized by the recombinase enzyme of interest. As outlined above, many recombination systems are known in the art and one of skill will recognize the appropriate recombination site to be used with the recombinase of interest.

Non-limiting examples of recombination sites include FRT sites including, for example, the native FRT site (FRT1, SEQ ID NO:37), and various functional variants of FRT, including but not limited to, FRT5 (SEQ ID NO:38), FRT6 (SEQ ID NO:39), FRT7 (SEQ ID NO:40), FRT12 (SEQ ID NO: 41), and FRT87 (SEQ ID NO:42). See, for example, International Application Publication Nos. WO 03/054189, WO 02/00900, and WO 01/23545; and Schlake et al. (1994) Biochemistry 33:12745-12751, each of which is herein incorporated by reference. Recombination sites from the Cre/Lox site-specific recombination system can be used. Such recombination sites include, for example, native LOX sites and various functional variants of LOX.

In some embodiments, the recombination site is a functional variant of a FRT site or functional variant of a LOX site, any combination thereof, or any other combination of recombinogenic or non-recombinogenic recombination sites known. Functional variants include chimeric recombination sites, such as an FRT site fused to a LOX site (see, for example, Luo et al. (2007) Plant Biotech J 5:263-274, which is herein incorporated by reference in its entirety). Functional variants also include minimal sites (FRT and/or LOX alone or in combination). The minimal native FRT recombination site (SEQ ID NO: 37) has been characterized and comprises a series of domains comprising a pair of 11 base pair symmetry elements, which are the FLP binding sites; the 8 base pair core, or spacer, region; and the polypyrimidine tracts. In some embodiments, at least one modified FRT recombination site is used. Modified or variant FRT recombination sites are sites having mutations such as alterations, additions, or deletions in the sequence. The modifications include sequence modification at any position, including but not limited to, a modification in at least one of the 8 base pair spacer domain, a symmetry element, and/or a polypyrimidine tract. FRT variants include minimal sites (see, e.g., Broach et al. (1982) Cell 29:227-234; Senecoff et al. (1985) Proc Natl Acad Sci USA 82:7270-7274; Gronostajski & Sadowski (1985) J Biol Chem 260:12320-12327; Senecoff et al. (1988) J Mol Biol 201:405-421; and International Application Publication No. WO99/25821), and sequence variants (see, for example, Schlake & Bode (1994) Biochemistry 33:12746-12751; Seibler & Bode (1997) Biochemistry 36:1740-1747; Umlauf & Cox (1988) EMBO J 7:1845-1852; Senecoff et al. (1988) J Mol Biol 201:405-421; Voziyanov et al. (2002) Nucleic Acids Res 30:7; International Application Publication Nos. WO 07/011,733, WO 99/25854, WO 99/25840, WO 99/25855, WO 99/25853 and WO 99/25821; and U.S. Pat. Nos. 7,060,499 and 7,476,539; each of which are herein incorporated by reference in its entirety).

An analysis of the recombination activity of variant LOX sites is presented in Lee et al. (1998) Gene 216:55-65 and in U.S. Pat. No. 6,465,254. Also, see for example, Huang et al. (1991) Nucleic Acids Res 19:443-448; Sadowski (1995) In Progress in Nucleic Acid Research and Molecular Biology Vol. 51, pp. 53-91; U.S. Pat. No. 6,465,254; Cox (1989) In Mobile DNA, Berg and Howe (eds) American Society of Microbiology, Washington D.C., pp. 116-670; Dixon et al. (1995) Mol Microbiol 18:449-458; Buchholz et al. (1996) Nucleic Acids Res 24:3118-3119; Kilby et al. (1993) Trends Genet 9:413-421; Rossant & Geagy (1995) Nat Med 1:592-594; Albert et al. (1995) Plant J 7:649-659; Bayley et al. (1992) Plant Mol Biol 18:353-361; Odell et al. (1990) Mol Gen Genet 223:369-378; Dale & Ow (1991) Proc Natl Acad Sci USA 88:10558-10562; Qui et al. (1994) Proc Natl Acad Sci USA 91:1706-1710; Stuurman et al. (1996) Plant Mol Biol 32:901-913; Dale et al. (1990) Gene 91:79-85; and International Application Publication No. WO 01/111058; each of which is herein incorporated by reference in its entirety.

Naturally occurring recombination sites or biologically active variants thereof are of use. Methods to determine if a modified recombination site is recombinogenic are known (see, for example, International Application Publication No. WO 07/011,733, which is herein incorporated by reference in its entirety). Variant recognition sites are known, see for example, Hoess et al. (1986) Nucleic Acids Res 14:2287-300; Albert et al. (1995) Plant J 7:649-59; Thomson et al. (2003) Genesis 36:162-7; Huang et al. (1991) Nucleic Acids Res 19:443-8; Siebler and Bode (1997) Biochemistry 36:1740-7; Schlake and Bode (1994) Biochemistry 33:12746-51; Thygarajan et al. (2001) Mol Cell Biol 21:3926-34; Umlauf and Cox (1988) EMBO J 7:1845-52; Lee and Saito (1998) Gene 216:55-65; International Application Publication Nos. WO 01/23545, WO 99/25851, WO 01/11058, WO 01/07572; and U.S. Pat. No. 5,888,732; each of which is herein incorporated by reference in its entirety.

The recombination sites employed in the methods and compositions can be identical or dissimilar sequences, so long as the sites are recombinogenic with respect to one another.

By “recombinogenic” is intended that the set of recombination sites (i.e., dissimilar or corresponding) are capable of recombining with one another. Alternatively, by “non-recombinogenic” is intended the set of recombination sites, in the presence of the appropriate recombinase, will not recombine with one another or recombination between the sites is minimal. Accordingly, it is recognized that any suitable set of recombinogenic recombination sites may be utilized, including a FRT site or functional variant thereof, a LOX site or functional variant thereof, any combination thereof, or any other combination of recombination sites known in the art.

In some embodiments, the recombination sites are asymmetric, and the orientation of any two sites relative to each other will determine the recombination reaction product. Directly repeated recombination sites are those recombination sites in a set of recombinogenic recombination sites that are arranged in the same orientation, such that recombination between these sites results in excision, rather than inversion, of the intervening DNA sequence. Inverted recombination sites are those recombination sites in a set of recombinogenic recombination sites that are arranged in the opposite orientation, so that recombination between these sites results in inversion, rather than excision, of the intervening DNA sequence. The presently disclosed polynucleotide constructs comprise recombination sites that are recombinogenic with one another and directly repeated so as to result in excision of the excision cassette.

The presently disclosed compositions and methods utilize at least one polynucleotide that confers herbicide tolerance. Tolerance to specific herbicides can be conferred by engineering genes into plants which encode appropriate herbicide metabolizing enzymes and/or insensitive herbicide targets. Such polypeptides are referred to as “herbicide tolerance polypeptides”. In some embodiments these enzymes, and the nucleic acids that encode them, originate from a plant. In other embodiments, they are derived from other organisms, such as microbes. See, e.g., Padgette et al. (1996) “New weed control opportunities: Development of soybeans with a Roundup Ready® gene” and Vasil (1996) “Phosphinothricin-resistant crops,” both in Herbicide-Resistant Crops, ed. Duke (CRC Press, Boca Raton, Fla.) pp. 54-84 and pp. 85-91.

An “herbicide” is a chemical that causes temporary or permanent injury to a plant. Non-limiting examples of herbicides that can be employed in the various methods and compositions of the invention are discussed in further detail elsewhere herein. A herbicide may be incorporated into the plant or plant part, or it may act on the plant or plant part without being incorporated into the plant or plant part. An “active ingredient” is the chemical in a herbicide formulation primarily responsible for its phytotoxicity and which is identified as the active ingredient on the product label. Product label information is available from the U.S. Environmental Protection Agency and is updated online at the url oaspub.epa.gov/pestlabl/ppls.own; product label information is also available online at the url www.cdms.net.

“Herbicide-tolerant” or “tolerant” in the context of herbicide or other chemical treatment as used herein means that a plant or plant part treated with a particular herbicide or class or subclass of herbicide or other chemical or class or subclass of other chemical will show no significant damage or less damage following that treatment in comparison to an appropriate control plant or plant part. A plant or plant part may be naturally tolerant to a particular herbicide or chemical, or a plant or plant part may be herbicide-tolerant as a result of human intervention such as, for example, breeding or genetic engineering. An “herbicide-tolerance polypeptide” is a polypeptide that confers herbicide tolerance on a plant or other organism expressing it (i.e., that makes a plant or other organism herbicide-tolerant), and an “herbicide-tolerance polynucleotide” is a polynucleotide that encodes a herbicide-tolerance polypeptide. For example, a sulfonylurea-tolerance polypeptide is one that confers tolerance to sulfonylurea herbicides on a plant or other organism that expresses it, an imidazolinone-tolerance polypeptide is one that confers tolerance to imidazolinone herbicides on a plant or other organism that expresses it; and a glyphosate-tolerance polypeptide is one that confers tolerance to glyphosate on a plant or other organism that expresses it.

Thus, a plant or plant part is tolerant to a herbicide or other chemical if it shows damage in comparison to an appropriate control plant or plant part that is less than the damage exhibited by the control plant or plant part by at least 5%, 10%, 15%, 20%, 25%, 30%, 35%, 40%, 45%, 50%, 55%, 60%, 65%, 70%, 75%, 80%, 90%, 100%, 150%, 200%, 250%, 300%, 400%, 500%, 600%, 700%, 800%, 900%, or 1000% or more. In this manner, a plant or plant part that is tolerant to a herbicide or other chemical shows “improved tolerance” in comparison to an appropriate control plant or plant part. Damage resulting from herbicide or other chemical treatment is assessed by evaluating any parameter of plant growth or well-being deemed suitable by one of skill in the art. Damage can be assessed by visual inspection and/or by statistical analysis of suitable parameters of individual plants or plant parts or of a group of plants or plant parts. Thus, damage may be assessed by evaluating, for example, parameters such as plant height, plant weight, leaf color, leaf length, flowering, fertility, silking, yield, seed production, and the like. Damage may also be assessed by evaluating the time elapsed to a particular stage of development (e.g., silking, flowering, or pollen shed) or the time elapsed until a plant has recovered from treatment with a particular chemical and/or herbicide.

In making such assessments, particular values may be assigned to particular degrees of damage so that statistical analysis or quantitative comparisons may be made. The use of ranges of values to describe particular degrees of damage is known in the art, and any suitable range or scale may be used. For example, herbicide injury scores (also called tolerance scores) can be assigned as set forth in Table 2. In this scale, a rating of 9 indicates that a herbicide treatment had no effect on a crop, i.e., that no crop reduction or injury was observed following the herbicide treatment. Thus, in this scale, a rating of 9 indicates that the crop exhibited no damage from the herbicide and therefore that the crop is tolerant to the herbicide. As indicated above, herbicide tolerance is also indicated by other ratings in this scale where an appropriate control plant exhibits a lower score on the scale, or where a group of appropriate control plants exhibits a statistically lower score in response to a herbicide treatment than a group of subject plants.

TABLE 2
Herbicide injury scale (1 to 9 scale scoring system).

| | Main | |
|---|---|---|
| Rating | categories | Detailed description |
| 9 | No Effect | No crop reduction or injury |
| 8 | Slight | Slight crop discoloration or stunting |
| 7 | Effect | Some crop discoloration, stunting, or stunt loss |
| 6 | | Crop injury more pronounced, but not lasting |
| 5 | Moderate | Moderate injury, crop usually recovers |
| 4 | Effect | Crop injury more lasting, recovery doubtful |
| 3 | | Lasting crop injury, no recovery |

A herbicide does not “significantly damage” a plant or plant part when it either has no effect on a plant or plant part or when it has some effect on a plant or plant part from which the plant later recovers, or when it has an effect which is detrimental but which is offset, for example, by the impact of the particular herbicide on weeds. Thus, for example, a plant or plant part is not “significantly damaged by” a herbicide or other treatment if it exhibits less than 50%, 40%, 30%, 25%, 20%, 15%, 10%, 9%, 8%, 7%, 6%, 5%, 4%, 3%, 2%, or 1% decrease in at least one suitable parameter that is indicative of plant health and/or productivity in comparison to an appropriate control plant or plant part (e.g., an untreated plant or plant part). Suitable parameters that are indicative of plant health and/or productivity include, for example, plant height, plant weight, leaf length, time elapsed to a particular stage of development, flowering, yield, seed production, and the like. The evaluation of a parameter can be by visual inspection and/or by statistical analysis of any suitable parameter. Comparison may be made by visual inspection and/or by statistical analysis. Accordingly, a plant or plant part is not “significantly damaged by” a herbicide or other treatment if it exhibits a decrease in at least one parameter but that decrease is temporary in nature and the plant or plant part recovers fully within 1 week, 2 weeks, 3 weeks, 4 weeks, or 6 weeks.

Conversely, a plant or plant part is significantly damaged by a herbicide or other treatment if it exhibits more than a 50%, 60%, 70%, 80%, 90%, 100%, 110%, 120%, 150%, 170% decrease in at least one suitable parameter that is indicative of plant health and/or productivity in comparison to an appropriate control plant or plant part. Thus, a plant or plant part is significantly damaged if it exhibits a decrease in at least one parameter and the plant or plant part does not recover fully within 1 week, 2 weeks, 3 weeks, 4 weeks, or 6 weeks.

Damage resulting from a herbicide or other chemical treatment of a plant or plant part can be assessed by visual inspection by one of skill in the art and can be evaluated by statistical analysis of suitable parameters. The plant or plant part being evaluated is referred to as the “test plant” or “test plant part.” Typically, an appropriate control plant or plant part is one that expresses the same herbicide-tolerance polypeptide(s) as the plant or plant part being evaluated for herbicide tolerance (i.e., the “test plant”) but that has not been treated with herbicide. In some circumstances, the control plant or plant part is one that has been subjected to the same herbicide treatment as the plant or plant part being evaluated (i.e., the test plant or plant part) but that does not express the enzyme intended to provide tolerance to the herbicide of interest in the test plant or plant part. One of skill in the art will be able to design, perform, and evaluate a suitable controlled experiment to assess the herbicide tolerance of a plant or plant part of interest, including the selection of appropriate test plants or plant part, control plants or plant part, and treatments.

Damage caused by a herbicide or other chemical can be assessed at various times after a plant or plant part has been contacted with a herbicide, although in some embodiments, assessment of the plant or plant part for herbicide tolerance occurs during or after rooting/regeneration of the plant or plant part. Often, damage is assessed at about the time that the control plant or plant part exhibits maximum damage. Sometimes, damage is assessed after a period of time in which a control plant or plant part that was not treated with herbicide has measurably grown and/or developed in comparison to the size or stage at which the treatment was administered. Damage can be assessed at various times, for example, at 12 hours or at 1, 2, 3, 4, 5, 6, 7, 8, 9, 10, 11, 12, 13, 14 days, or three weeks, four weeks, or longer after the test plant or plant part was treated with herbicide. Any time of assessment is suitable as long as it permits detection of a difference in response to a treatment of test and control plants or plant parts.

Thus, as used herein, a “test plant” or “test plant part” is one which has been transformed with the presently disclosed polynucleotide constructs or is a plant or plant part which is descended from a plant or plant part so altered and which comprises the herbicide tolerance polynucleotide.

A “control” or “control plant” or “control plant part” provides a reference point for measuring changes in phenotype of the subject plant or plant part, and may be any suitable plant or plant part. A control plant or plant part may comprise, for example: (a) a wild-type plant or plant part, i.e., an untransformed plant of the same genotype as the test plant or plant part prior to transformation; (b) a plant or plant part of the same genotype as the starting material but which has been transformed with a null construct (i.e., with a construct which has no known effect on the trait of interest, such as a construct comprising a marker gene); (c) a plant or plant part which is a non-transformed segregant among progeny of a subject plant or plant part; (d) a plant or plant part which is genetically identical to the subject plant or plant part but which is not exposed to the same treatment (e.g., herbicide treatment) as the subject plant or plant part; (e) the subject plant or plant part itself, under conditions in which the herbicide tolerance polynucleotide is not expressed; or (f) the subject plant or plant part itself, under conditions in which it has not been exposed to a particular treatment such as, for example, a herbicide or combination of herbicides and/or other chemicals. In some instances, an appropriate control maize plant or plant part comprises a NK603 event (Nielson et al. (2004) European Food Research and Technology 219:421-427 and Ridley et al. (2002) Journal of Agriculture and Food Chemistry 50: 7235-7243), an elite stiff stalk inbred plant, a P3162 plant (Pioneer Hi-Bred International), a 39T66 plant (Pioneer Hi-Bred International), or a 34M91 plant (Pioneer Hi-Bred International). In some instances, an appropriate control soybean plant or plant part is a “Jack” soybean plant (Illinois Foundation Seed, Champaign, Illinois).

The herbicide tolerance polypeptides used in the presently disclosed compositions and methods can confer tolerance to any respective herbicide. In some embodiments, the herbicide tolerance polypeptide confers tolerance to a herbicide selected from the group consisting of glyphosate, an ALS inhibitor (e.g., a sulfonylurea), an acetyl Co-A carboxylase inhibitor, a synthetic auxin, a protoporphyrinogen oxidase (PPO) inhibitor herbicide, a pigment synthesis inhibitor herbicide, a phosphinothricin acetyltransferase or a phytoene desaturase inhibitor, a glutamine synthase inhibitor, a hydroxyphenylpyruvatedioxygenase inhibitor, and a protoporphyrinogen oxidase inhibitor.

One herbicide which has been studied extensively is N-phosphonomethylglycine, commonly referred to as glyphosate. Glyphosate is a broad spectrum herbicide that kills both broadleaf and grass-type plants due to inhibition of the enzyme 5-enolpyruvylshikimate-3-phosphate synthase (also referred to as “EPSP synthase” or “EPSPS”), an enzyme which is part of the biosynthetic pathway for the production of aromatic amino acids, hormones, and vitamins. Glyphosate-resistant transgenic plants have been produced which exhibit a commercially viable level of glyphosate resistance due to the introduction of a modified Agrobacterium CP4 EPSPS. This modified enzyme is targeted to the chloroplast where, even in the presence of glyphosate, it continues to synthesize EPSP from phosphoenolpyruvic acid (“PEP”) and shikimate-3-phosphate. CP4 glyphosate-resistant soybean transgenic plants are presently in commercial use (e.g., as sold by Monsanto under the name “Roundup Ready®”).

In some embodiments, the presently disclosed methods and compositions utilize a polynucleotide that encodes a herbicide tolerance polypeptide that confers tolerance to glyphosate. Various sequences which confer tolerance to glyphosate can be employed in the presently disclosed methods and compositions. In some embodiments, the herbicide tolerance polypeptide that confers resistance to glyphosate has glyphosate transferase activity. As used herein, a “glyphosate transferase” polypeptide has the ability to transfer the acetyl group from acetyl CoA to the N of glyphosate, transfer the propionyl group of propionyl CoA to the N of glyphosate, or to catalyze the acetylation of glyphosate analogs and/or glyphosate metabolites, e.g., aminomethylphosphonic acid. Methods to assay for this activity are disclosed, for example, in U.S. Publication No. 2003/0083480, U.S. Publication No. 2004/0082770, and U.S. Pat. No. 7,405,074, WO2005/012515, WO2002/36782 and WO2003/092360. In one embodiment, the transferase polypeptide comprises a glyphosate-N-acetyltransferase “GLYAT” polypeptide.

As used herein, a GLYAT polypeptide or enzyme comprises a polypeptide which has glyphosate-N-acetyltransferase activity (“GLYAT” activity), i.e., the ability to catalyze the acetylation of glyphosate. In specific embodiments, a polypeptide having glyphosate-N-acetyltransferase activity can transfer the acetyl group from acetyl CoA to the N of glyphosate. In addition, some GLYAT polypeptides transfer the propionyl group of propionyl CoA to the N of glyphosate. Some GLYAT polypeptides are also capable of catalyzing the acetylation of glyphosate analogs and/or glyphosate metabolites, e.g., aminomethylphosphonic acid. GLYAT polypeptides are characterized by their structural similarity to one another, e.g., in terms of sequence similarity when the GLYAT polypeptides are aligned with one another. Exemplary GLYAT polypeptides and the polynucleotides encoding them are known in the art and particularly disclosed, for example, in U.S. App. Publ. No. 2003/0083480, and U.S. Pat. Nos. 7,462,481, 7,531,339, 7,622,641, and U.S. Pat. No. 7,405,074, each of which is herein incorporated by reference in its entirety. In some embodiments, GLYAT polypeptides used in the presently disclosed methods and compositions comprise the amino acid sequence set forth in: SEQ ID NO: 43, 44, 45, 46, 48, or 50. In some embodiments, the GLYAT polynucleotide that encodes the GLYAT polypeptide that is used in the presently disclosed methods and compositions are set forth in SEQ ID NO: 47 or 49. As discussed in further detail elsewhere herein, the use of fragments and variants of GLYAT polynucleotides and other known herbicide-tolerance polynucleotides and polypeptides encoded thereby is also encompassed by the present invention.

Active variants of SEQ ID NOS: 43, 44, 45, 46, 48, or 50 which retain glyphosate N-acetyltranserase activity include sequences which generate a similarity score of at least 430 using the BLOSUM62 matrix, a gap existence penalty of 11, and a gap extension penalty of 1 when optimally aligned with any one of SEQ ID NO. Some aspects of the invention pertain to GAT polypeptides comprising an amino acid sequence that can be optimally aligned with an amino acid sequence selected from the group consisting of SEQ ID NOS: 43, 44, 45, 46, 48, and 50 to generate a similarity score of at least 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, or 760 using the BLOSUM62 matrix, a gap existence penalty of 11, and a gap extension penalty of 1. Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences.

Plants expressing GLYAT that have been treated with glyphosate contain the glyphosate metabolite N-acetylglyphosate (“NAG”). The presence of N-acetylglyphosate can serve as a diagnostic marker for the presence of an active GLYAT gene in a plant and can be evaluated by methods known in the art, for example, by mass spectrometry or by immunoassay. Generally, the level of NAG in a plant containing a GLYAT gene that has been treated with glyphosate is correlated with the activity of the GLYAT gene and the amount of glyphosate with which the plant has been treated.

Polynucleotides that encode glyphosate tolerance polypeptides that can be used in the presently disclosed methods and compositions include those that encode a glyphosate oxido-reductase enzyme as described more fully in U.S. Pat. Nos. 5,776,760 and 5,463,175, which are incorporated herein by reference in their entireties for all purposes.

Other herbicides commonly used for commercial crop production include glufosinate (phosphinothricin) and acetolactate synthase (ALS) chemistry such as the sulfonylurea herbicides. Glufosinate is a broad spectrum herbicide which acts on the chloroplast glutamate synthase enzyme. Glufosinate-tolerant transgenic plants have been produced which carry the bar gene from Streptomyces hygroscopicus. The enzyme encoded by the bar gene has N-acetylation activity and modifies and detoxifies glufosinate. Glufosinate-tolerant plants are presently in commercial use (e.g., as sold by Bayer under the name “Liberty Link®”). As described elsewhere herein, sulfonylurea herbicides inhibit growth of higher plants by blocking acetolactate synthase (ALS). Plants containing particular mutations in ALS are tolerant to the ALS herbicides including sulfonylureas.

In some embodiments, the herbicide tolerance polypeptide that is utilized in the presently disclosed methods and compositions is an ALS inhibitor-tolerance polypeptide. As used herein, an “ALS inhibitor-tolerance polypeptide” comprises any polypeptide which when expressed in a plant confers tolerance to at least one ALS inhibitor. A variety of ALS inhibitors are known and include, for example, sulfonylurea, imidazolinone, triazolopyrimidines, pryimidinyoxy(thio)benzoates, and/or sulfonylaminocarbonyltriazolinone herbicides. Additional ALS inhibitors are known and are disclosed elsewhere herein. It is known in the art that ALS mutations fall into different classes with regard to tolerance to sulfonylureas, imidazolinones, triazolopyrimidines, and pyrimidinyl(thio)benzoates, including mutations having the following characteristics: (1) broad tolerance to all four of these groups; (2) tolerance to imidazolinones and pyrimidinyl(thio)benzoates; (3) tolerance to sulfonylureas and triazolopyrimidines; and (4) tolerance to sulfonylureas and imidazolinones.

Various ALS inhibitor-tolerance polypeptides can be employed. In some embodiments, the ALS inhibitor-tolerance polynucleotides contain at least one nucleotide mutation resulting in one amino acid change in the ALS polypeptide. In specific embodiments, the change occurs in one of seven substantially conserved regions of acetolactate synthase. See, for example, Hattori et al. (1995) Molecular Genetics and Genomes 246:419-425; Lee et al. (1998) EMBO Journal 7:1241-1248; Mazur et al. (1989) Ann. Rev. Plant Phys. 40:441-470; and U.S. Pat. No. 5,605,011, each of which is incorporated by reference in their entirety. The ALS inhibitor-tolerance polypeptide can be encoded by, for example, the SuRA or SuRB locus of ALS. In specific embodiments, the ALS inhibitor-tolerance polypeptide comprises the C3 ALS mutant, the HRA ALS mutant, the S4 mutant or the S4/HRA mutant or any combination thereof. Different mutations in ALS are known to confer tolerance to different herbicides and groups (and/or subgroups) of herbicides; see, e.g., Tranel and Wright (2002) Weed Science 50:700-712. See also, U.S. Pat. Nos. 5,605,011, 5,378,824, 5,141,870, 5,013,659, and U.S. Pat. No. 7,622,641, each of which is herein incorporated by reference in their entirety. See also, SEQ ID NO:51 comprising a soybean HRA sequence; SEQ ID NO:52 comprising a maize HRA sequence; and SEQ ID NO:53 comprising an Arabidopsis HRA sequence. The HRA mutation in ALS finds particular use in one embodiment of the invention. The mutation results in the production of an acetolactate synthase polypeptide which is resistant to at least one ALS inhibitor chemistry in comparison to the wild-type protein. For example, a plant expressing an ALS inhibitor-tolerant polypeptide may be tolerant of a dose of sulfonylurea, imidazolinone, triazolopyrimidines, pryimidinyloxy(thio)benzoates, and/or sulfonylaminocarbonyltriazolinone herbicide that is at least 2, 3, 4, 5, 6, 7, 8, 9, 10, 15, 20, 25, 30, 35, 40, 50, 70, 80, 100, 125, 150, 200, 500, or 1000 times higher than a dose of the herbicide that would cause damage to an appropriate control plant. In some embodiments, an ALS inhibitor-tolerant polypeptide comprises a number of mutations.

In some embodiments, the ALS inhibitor-tolerance polypeptide confers tolerance to sulfonylurea and imidazolinone herbicides. Sulfonylurea and imidazolinone herbicides inhibit growth of higher plants by blocking acetolactate synthase (ALS), also known as, acetohydroxy acid synthase (AHAS). For example, plants containing particular mutations in ALS (e.g., the S4 and/or HRA mutations) are tolerant to sulfonylurea herbicides. The production of sulfonylurea-tolerant plants and imidazolinone-tolerant plants is described more fully in U.S. Pat. Nos. 5,605,011; 5,013,659; 5,141,870; 5,767,361; 5,731,180; 5,304,732; 4,761,373; 5,331,107; 5,928,937; and U.S. Pat. No. 5,378,824; and international publication WO 96/33270, which are incorporated herein by reference in their entireties for all purposes. In specific embodiments, the ALS inhibitor-tolerance polypeptide comprises a sulfonamide-tolerant acetolactate synthase (otherwise known as a sulfonamide-tolerant acetohydroxy acid synthase) or an imidazolinone-tolerant acetolactate synthase (otherwise known as an imidazolinone-tolerant acetohydroxy acid synthase).

Often, a herbicide-tolerance polynucleotide that confers tolerance to a particular herbicide or other chemical or a plant expressing it will also confer tolerance to other herbicides or chemicals in the same class or subclass, for example, a class or subclass set forth in Table 3.

TABLE 3
Abbreviated version of HRAC Herbicide Classification

```
I. ALS Inhibitors (WSSA Group 2)
A. Sulfonylureas
  1. Azimsulfuron
  2. Chlorimuron-ethyl
  3. Metsulfuron-methyl
  4. Nicosulfuron
  5. Rimsulfuron
  6. Sulfometuron-methyl
  7. Thifensulfuron-methyl
  8. Tribenuron-methyl
  9. Amidosulfuron
10. Bensulfuron-methyl
11. Chlorsulfuron
12. Cinosulfuron
13. Cyclosulfamuron
14. Ethametsulfuron-methyl
15. Ethoxysulfuron
16. Flazasulfuron
17. Flupyrsulfuron-methyl
18. Foramsulfuron
19. Imazosulfuron
20. Iodosulfuron-methyl
21. Mesosulfuron-methyl
22. Oxasulfuron
23. Primisulfuron-methyl
24. Prosulfuron
25. Pyrazosulfuron-ethyl
26. Sulfosulfuron
27. Triasulfuron
28. Trifloxysulfuron
29. Triflusulfuron-methyl
30. Tritosulfuron
31. Halosulfuron-methyl
32. Flucetosulfuron
B. Sulfonylaminocarbonyltriazolinones
  1. Flucarbazone
  2. Procarbazone
C. Triazolopyrimidines
  1. Cloransulam-methyl
  2. Flumetsulam
  3. Diclosulam
  4. Florasulam
  5. Metosulam
  6. Penoxsulam
  7. Pyroxsulam
D. Pyrimidinyloxy(thio)benzoates
  1. Bispyribac
  2. Pyriftalid
  3. Pyribenzoxim
  4. Pyrithiobac
  5. Pyriminobac-methyl
E. Imidazolinones
  1. Imazapyr
  2. Imazethapyr
  3. Imazaquin
  4. Imazapic
  5. Imazamethabenz-methyl
  6. Imazamox
II. Other Herbicides—Active Ingredients/Additional Modes of Action
A. Inhibitors of Acetyl CoA carboxylase (ACCase) (WSSA Group 1)
  1. Aryloxyphenoxypropionates (‘FOPS’)
a. Quizalofop-P-ethyl
b. Diclofop-methyl
c. Clodinafop-propargyl
d. Fenoxaprop-P-ethyl
e. Fluazifop-P-butyl
f. Propaquizafop
g. Haloxyfop-P-methyl
h. Cyhalofop-butyl
i. Quizalofop-P-ethyl
  2. Cyclohexanediones (‘DIMS’)
a. Alloxydim
b. Butroxydim
c. Clethodim
d. Cycloxydim
e. Sethoxydim
f. Tepraloxydim
g. Tralkoxydim
B. Inhibitors of Photosystem II—HRAC Group C1/WSSA Group 5
  1. Triazines
a. Ametryne
b. Atrazine
c. Cyanazine
d. Desmetryne
e. Dimethametryne
f. Prometon
g. Prometryne
h. Propazine
i. Simazine
j. Simetryne
k. Terbumeton
l. Terbuthylazine
m. Terbutryne
n. Trietazine
  2. Triazinones
a. Hexazinone
b. Metribuzin
c. Metamitron
  3. Triazolinone
a. Amicarbazone
  4. Uracils
a. Bromacil
b. Lenacil
c. Terbacil
  5. Pyridazinones
a. Pyrazon
  6. Phenyl carbamates
a. Desmedipham
b. Phenmedipham
C. Inhibitors of Photosystem II—HRAC Group C2/WSSA Group 7
  1. Ureas
a. Fluometuron
b. Linuron
c. Chlorobromuron
d. Chlorotoluron
e. Chloroxuron
f. Dimefuron
g. Diuron
h. Ethidimuron
i. Fenuron
j. Isoproturon
k. Isouron
l. Methabenzthiazuron
m. Metobromuron
n. Metoxuron
o. Monolinuron
p. Neburon
q. Siduron
r. Tebuthiuron
  2. Amides
a. Propanil
b. Pentanochlor
D. Inhibitors of Photosystem II—HRAC Group C3/WSSA Group 6
  1. Nitriles
a. Bromofenoxim
b. Bromoxynil
c. Ioxynil
  2. Benzothiadiazinone (Bentazon)
a. Bentazon
  3. Phenylpyridazines
a. Pyridate
b. Pyridafol
E. Photosystem-I-electron diversion (Bipyridyliums) (WSSA Group 22)
  1. Diquat
  2. Paraquat
F. Inhibitors of PPO (protoporphyrinogen oxidase) (WSSA Group 14)
  1. Diphenylethers
a. Acifluorfen-Na
b. Bifenox
c. Chlomethoxyfen
d. Fluoroglycofen-ethyl
e. Fomesafen
f. Halosafen
g. Lactofen
h. Oxyfluorfen
  2. Phenylpyrazoles
a. Fluazolate
b. Pyraflufen-ethyl
  3. N-phenylphthalimides
a. Cinidon-ethyl
b. Flumioxazin
c. Flumiclorac-pentyl
  4. Thiadiazoles
a. Fluthiacet-methyl
b. Thidiazimin
  5. Oxadiazoles
a. Oxadiazon
b. Oxadiargyl
  6. Triazolinones
a. Carfentrazone-ethyl
b. Sulfentrazone
  7. Oxazolidinediones
a. Pentoxazone
  8. Pyrimidindiones
a. Benzfendizone
b. Butafenicil
  9. Others
a. Pyrazogyl
b. Profluazol
G. Bleaching: Inhibition of carotenoid biosynthesis at the phytoene
desaturase step (PDS) (WSSA Group 12)
  1. Pyridazinones
a. Norflurazon
  2. Pyridinecarboxamides
a. Diflufenican
b. Picolinafen
  3. Others
a. Beflubutamid
b. Fluridone
c. Flurochloridone
H. Bleaching: Inhibition of 4-hydroxyphenyl-pyruvate-dioxygenase
(4-HPPD) (WSSA Group 28)
  1. Triketones
a. Mesotrione
b. Sulcotrione
  2. Isoxazoles
a. Isoxachlortole
b. Isoxaflutole
  3. Pyrazoles
a. Benzofenap
b. Pyrazoxyfen
c. Pyrazolynate
  4. Others
a. Benzobicyclon
I. Bleaching: Inhibition of carotenoid biosynthesis (unknown target)
(WSSA Group 11 and 13)
  1. Triazoles (WSSA Group 11)
a. Amitrole
  2. Isoxazolidinones (WSSA Group 13)
a. Clomazone
  3. Ureas
a. Fluometuron
  3. Diphenylether
a. Aclonifen
J. Inhibition of EPSP Synthase
  1. Glycines (WSSA Group 9)
a. Glyphosate
b. Sulfosate
K. Inhibition of glutamine synthetase
  1. Phosphinic Acids
a. Glufosinate-ammonium
b. Bialaphos
L. Inhibition of DHP (dihydropteroate) synthase (WSSA Group 18)
  1 Carbamates
a. Asulam
M. Microtubule Assembly Inhibition (WSSA Group 3)
  1. Dinitroanilines
a. Benfluralin
b. Butralin
c. Dinitramine
d. Ethalfluralin
e. Oryzalin
f. Pendimethalin
g. Trifluralin
  2. Phosphoroamidates
a. Amiprophos-methyl
b. Butamiphos
  3. Pyridines
a. Dithiopyr
b. Thiazopyr
  4. Benzamides
a. Pronamide
b. Tebutam
  5. Benzenedicarboxylic acids
a. Chlorthal-dimethyl
N. Inhibition of mitosis/microtubule organization WSSA Group 23)
  1. Carbamates
a. Chlorpropham
b. Propham
c. Carbetamide
O. Inhibition of cell division (Inhibition of very long chain fatty acids
as proposed mechanism; WSSA Group 15)
  1. Chloroacetamides
a. Acetochlor
b. Alachlor
c. Butachlor
d. Dimethachlor
e. Dimethanamid
f. Metazachlor
g. Metolachlor
h. Pethoxamid
i. Pretilachlor
j. Propachlor
k. Propisochlor
l. Thenylchlor
  2. Acetamides
a. Diphenamid
b. Napropamide
c. Naproanilide
  3. Oxyacetamides
a. Flufenacet
b. Mefenacet
  4. Tetrazolinones
a. Fentrazamide
  5. Others
a. Anilofos
b. Cafenstrole
c. Indanofan
d. Piperophos
P. Inhibition of cell wall (cellulose) synthesis
  1. Nitriles (WSSA Group 20)
a. Dichlobenil
b. Chlorthiamid
  2. Benzamides (isoxaben (WSSA Group 21))
a. Isoxaben
  3. Triazolocarboxamides (flupoxam)
a. Flupoxam
Q. Uncoupling (membrane disruption): (WSSA Group 24)
  1. Dinitrophenols
a. DNOC
b. Dinoseb
c. Dinoterb
R. Inhibition of Lipid Synthesis by other than ACC inhibition
  1. Thiocarbamates (WSSA Group 8)
a. Butylate
b. Cycloate
c. Dimepiperate
d. EPTC
e. Esprocarb
f. Molinate
g. Orbencarb
h. Pebulate
i. Prosulfocarb
j. Benthiocarb
k. Tiocarbazil
l. Triallate
m. Vernolate
  2. Phosphorodithioates
a. Bensulide
  3. Benzofurans
a. Benfuresate
b. Ethofumesate
  4. Halogenated alkanoic acids (WSSA Group 26)
a. TCA
b. Dalapon
c. Flupropanate
S. Synthetic auxins (IAA-like) (WSSA Group 4)
  1. Phenoxycarboxylic acids
a. Clomeprop
b. 2,4-D
c. Mecoprop
  2. Benzoic acids
a. Dicamba
b. Chloramben
c. TBA
  3. Pyridine carboxylic acids
a. Clopyralid
b. Fluroxypyr
c. Picloram
d. Tricyclopyr
  4. Quinoline carboxylic acids
a. Quinclorac
b. Quinmerac
  5. Others (benazolin-ethyl)
a. Benazolin-ethyl
T. Inhibition of Auxin Transport
  1. Phthalamates; semicarbazones (WSSA Group 19)
a. Naptalam
b. Diflufenzopyr-Na
U. Other Mechanism of Action
  1. Arylaminopropionic acids
a. Flamprop-M-methyl/-isopropyl
  2. Pyrazolium
a. Difenzoquat
  3. Organoarsenicals
a. DSMA
b. MSMA
  4. Others
a. Bromobutide
b. Cinmethylin
c. Cumyluron
d. Dazomet
e. Daimuron-methyl
f. Dimuron
g. Etobenzanid
h. Fosamine
i. Metam
j. Oxaziclomefone
k. Oleic acid
l. Pelargonic acid
m. Pyributicarb
```

The presently disclosed methods and compositions can utilize multiple herbicide tolerance polynucleotides. That is, the presently disclosed polynucleotide constructs can comprise more than one coding polynucleotide for a herbicide tolerance polypeptide. In some embodiments, the polynucleotide construct comprises more than one polynucleotide that encodes the same type of herbicide tolerance polypeptide (i.e., more than one GLYAT). In other embodiments, the polynucleotide constructs comprise more than one herbicide-tolerance coding polynucleotide, wherein each of the coding polynucleotides encodes for a distinct type of herbicide tolerance polypeptide (of a different class or subclass). In some embodiments, the polynucleotide construct comprises at least a first and a second polynucleotide encoding a herbicide tolerance polypeptide, wherein the first and the second polynucleotide encodes a first and a second herbicide tolerance polypeptide that confer tolerance to a first and a second herbicide, wherein the first and second herbicide have different mechanisms of action.

In some of those embodiments wherein the presently disclosed polynucleotide constructs comprise at least two herbicide tolerance polynucleotides, at least two herbicide tolerance polynucleotides are located outside of the excision cassette. In other embodiments, the polynucleotide construct comprises a herbicide tolerance polynucleotide outside of the excision cassette that becomes operably linked to its promoter upon excision of the excision cassette and a second herbicide tolerance polypeptide within the excision cassette.

In some embodiments, the presently disclosed methods and compositions utilize polynucleotides that confer tolerance to glyphosate and at least one ALS inhibitor herbicide. In other embodiments, the presently disclosed methods and compositions utilize polynucleotides that confer tolerance to glyphosate and at least one ALS inhibitor herbicide, as well as, tolerance to at least one additional herbicide.

In addition to glyphosate and ALS inhibitors, the presently disclosed polynucleotide constructs can comprise polynucleotides that encode herbicide tolerance polypeptides that confer tolerance to other types of herbicides. Such additional herbicides, include but are not limited to, an acetyl Co-A carboxylase inhibitor such as quizalofop-P-ethyl, a synthetic auxin such as quinclorac, a protoporphyrinogen oxidase (PPO) inhibitor herbicide (such as sulfentrazone), a pigment synthesis inhibitor herbicide such as a hydroxyphenylpyruvate dioxygenase inhibitor (e.g., mesotrione or sulcotrione), a phosphinothricin acetyltransferase or a phytoene desaturase inhibitor like diflufenican or pigment synthesis inhibitor.

In some embodiments, the presently disclosed polynucleotide constructs comprise polynucleotides encoding polypeptides conferring tolerance to herbicides which inhibit the enzyme glutamine synthase, such as phosphinothricin or glufosinate (e.g., the bar gene or pat gene). Glutamine synthetase (GS) appears to be an essential enzyme necessary for the development and life of most plant cells, and inhibitors of GS are toxic to plant cells. Glufosinate herbicides have been developed based on the toxic effect due to the inhibition of GS in plants. These herbicides are non-selective; that is, they inhibit growth of all the different species of plants present. The development of plants containing an exogenous phosphinothricin acetyltransferase is described in U.S. Pat. Nos. 5,969,213; 5,489,520; 5,550,318; 5,874,265; 5,919,675; 5,561,236; 5,648,477; 5,646,024; 6,177,616; and U.S. Pat. No. 5,879,903, which are incorporated herein by reference in their entireties for all purposes. Mutated phosphinothricin acetyltransferase having this activity are also disclosed. In certain embodiments a maize-optimized PAT gene is used. In some of these embodiments, the maize-optimized PAT gene has the sequence set forth in SEQ ID NO: 54. In some embodiments, the PAT gene is used as a selectable marker as described elsewhere herein and is present within the excision cassette.

In still other embodiments, the presently disclosed polynucleotide constructs comprise polynucleotides encoding polypeptides conferring tolerance to herbicides which inhibit protox (protoporphyrinogen oxidase). Protox is necessary for the production of chlorophyll, which is necessary for all plant survival. The protox enzyme serves as the target for a variety of herbicidal compounds. These herbicides also inhibit growth of all the different species of plants present. The development of plants containing altered protox activity which are resistant to these herbicides are described in U.S. Pat. Nos. 6,288,306; 6,282,837; and U.S. Pat. No. 5,767,373; and international publication WO 01/12825, which are incorporated herein by reference in their entireties for all purposes.

In still other embodiments, the presently disclosed polynucleotide constructs may comprise polynucleotides encoding polypeptides involving other modes of herbicide resistance. For example, hydroxyphenylpyruvatedioxygenases are enzymes that catalyze the reaction in which para-hydroxyphenylpyruvate (HPP) is transformed into homogentisate. Molecules which inhibit this enzyme and which bind to the enzyme in order to inhibit transformation of the HPP into homogentisate are useful as herbicides. Plants more resistant to certain herbicides are described in U.S. Pat. Nos. 6,245,968; 6,268,549; and 6,069,115; and international publication WO 99/23886, which are incorporated herein by reference in their entireties for all purposes. Mutated hydroxyphenylpyruvatedioxygenase having this activity are also disclosed.

In some embodiments, the methods and compositions can further comprise at least one cell proliferation factor. Expression of a cell proliferation factor, such as babyboom can enhance the transformation frequency of otherwise recalcitrant plants or plant parts. A polynucleotide encoding a cell proliferation factor can be co-transformed into a plant or plant part with the presently disclosed polynucleotide constructs. In other embodiments, the presently disclosed polynucleotide constructs comprise at least one polynucleotide encoding a cell proliferation factor. In some of these embodiments, the at least one polynucleotide encoding a cell proliferation factor is located within the excision cassette of the polynucleotide construct, such that the polynucleotide is excised when the site-specific recombinase is expressed.

As used herein, a “cell proliferation factor” is a polypeptide or a polynucleotide capable of stimulating growth of a cell or tissue, including but not limited to promoting progression through the cell cycle, inhibiting cell death, such as apoptosis, stimulating cell division, and/or stimulating embryogenesis. The polynucleotides can fall into several categories, including but not limited to, cell cycle stimulatory polynucleotides, developmental polynucleotides, anti-apoptosis polynucleotides, hormone polynucleotides, or silencing constructs targeted against cell cycle repressors or pro-apoptotic factors. The following are provided as non-limiting examples of each category and are not considered a complete list of useful polynucleotides for each category: 1) cell cycle stimulatory polynucleotides including plant viral replicase genes such as RepA, cyclins, E2F, prolifera, cdc2 and cdc25; 2) developmental polynucleotides such as Lec1, Kn1 family, WUSCHEL, Zwille, BBM, Aintegumenta (ANT), FUS3, and members of the Knotted family, such as Kn1, STM, OSH1, and SbH1; 3) anti-apoptosis polynucleotides such as CED9, Bcl2, Bcl-X(L), Bcl-W, A1, McL-1, Mac1, Boo, and Bax-inhibitors; 4) hormone polynucleotides such as IPT, TZS, and CKI-1; and 5) silencing constructs targeted against cell cycle repressors, such as Rb, CK1, prohibitin, and wee1, or stimulators of apoptosis such as APAF-1, bad, bax, CED-4, and caspase-3, and repressors of plant developmental transitions, such as Pickle and WD polycomb genes including FIE and Medea. The polynucleotides can be silenced by any known method such as antisense, RNA interference, cosuppression, chimerplasty, or transposon insertion.

The polynucleotide encoding the cell proliferation factor may be native to the cell or heterologous. Any of a number of cell proliferation factors can be used. In certain embodiments, those cell proliferation factors that are capable of stimulating embryogenesis are used to enhance transformation efficiency. Such cell proliferation factors are referred to herein as embryogenesis-stimulating polypeptides and they include, but are not limited to, babyboom polypeptides.

In some embodiments, the cell proliferation factor is a member of the AP2/ERF family of proteins. The AP2/ERF family of proteins is a plant-specific class of putative transcription factors that regulate a wide variety of developmental processes and are characterized by the presence of an AP2 DNA binding domain that is predicted to form an amphipathic alpha helix that binds DNA (PFAM Accession PF00847). The AP2/ERF proteins have been subdivided into distinct subfamilies based on the presence of conserved domains. Initially, the family was divided into two subfamilies based on the number of DNA binding domains, with the ERF subfamily having one DNA binding domain, and the AP2 subfamily having 2 DNA binding domains. As more sequences were identified, the family was subsequently subdivided into five subfamilies: AP2, DREB, ERF, RAV, and others. (Sakuma et al. (2002) Biochem Biophys Res Comm 290:998-1009).

Members of the APETALA2 (AP2) family of proteins function in a variety of biological events, including but not limited to, development, plant regeneration, cell division, embryogenesis, and cell proliferation (see, e.g., Riechmann and Meyerowitz (1998) Biol Chem 379:633-646; Saleh and Pages (2003) Genetika 35:37-50 and Database of Arabidopsis Transciption Factors at daft.cbi.pku.edu.cn). The AP2 family includes, but is not limited to, AP2, ANT, Glossy15, AtBBM, BnBBM, and maize ODP2/BBM.

U.S. Application Publication No. 2011/0167516, which is herein incorporated by reference in its entirety, describes an analysis of fifty sequences with homology to a maize BBM sequence (also referred to as maize ODP2 or ZmODP2, the polynucleotide and amino acid sequence of the maize BBM is set forth in SEQ ID NO: 55 and 56, respectively; the polynucleotide and amino acid sequence of another ZmBBM is set forth in SEQ ID NO: 58 and 59, respectively). The analysis identified three motifs (motifs 4-6; set forth in SEQ ID NOs: 61-63), along with the AP2 domains (motifs 2 and 3; SEQ ID NOs: 64 and 65) and linker sequence that bridges the AP2 domains (motif 1; SEQ ID NO: 66), that are found in all of the BBM homologues. Thus, motifs 1-6 distinguish these BBM homologues from other AP2-domain containing proteins (e.g., WR1, AP2, and RAP2.7) and these BBM homologues comprise a subgroup of AP2 family of proteins referred to herein as the BBM/PLT subgroup. In some embodiments, the cell proliferation factor that is used in the methods and compositions is a member of the BBM/PLT group of AP2 domain-containing polypeptides. In these embodiments, the cell proliferation factor comprises two AP2 domains and motifs 4-6 (SEQ ID NOs: 61-63) or a fragment or variant thereof. In some of these embodiments, the AP2 domains have the sequence set forth in SEQ ID NOs: 64 and 65 or a fragment or variant thereof, and in particular embodiments, further comprises the linker sequence of SEQ ID NO: 66 or a fragment or variant thereof. In other embodiments, the cell proliferation factor comprises at least one of motifs 4-6 or a fragment or variant thereof, along with two AP2 domains, which in some embodiments have the sequence set forth in SEQ ID NO: 64 and/or 65 or a fragment or variant thereof, and in particular embodiments have the linker sequence of SEQ ID NO: 66 or a fragment or variant thereof. Based on the phylogenetic analysis, the subgroup of BBM/PLT polypeptides can be subdivided into the BBM, AIL6/7, PLT1/2, AIL1, PLT3, and ANT groups of polypeptides.

In some embodiments, the cell proliferation factor is a babyboom (BBM) polypeptide, which is a member of the AP2 family of transcription factors. The BBM protein from Arabidopsis (AtBBM) is preferentially expressed in the developing embryo and seeds and has been shown to play a central role in regulating embryo-specific pathways. Overexpression of AtBBM has been shown to induce spontaneous formation of somatic embryos and cotyledon-like structures on seedlings. See, Boutiler et al. (2002) The Plant Cell 14:1737-1749. The maize BBM protein also induces embryogenesis and promotes transformation (See, U.S. Pat. No. 7,579,529, which is herein incorporated by reference in its entirety). Thus, BBM polypeptides stimulate proliferation, induce embryogenesis, enhance the regenerative capacity of a plant, enhance transformation, and as demonstrated herein, enhance rates of targeted polynucleotide modification.

In some embodiments, the babyboom polypeptide comprises two AP2 domains and at least one of motifs 7 and 10 (set forth in SEQ ID NO: 67 and 68, respectively) or a variant or fragment thereof. In certain embodiments, the AP2 domains are motifs 2 and 3 (SEQ ID NOs: 64 and 65, respectively) or a fragment or variant thereof, and in particular embodiments, the babyboom polypeptide further comprises a linker sequence between AP2 domain 1 and 2 having motif 1 (SEQ ID NO: 66) or a fragment or variant thereof. In particular embodiments, the BBM polypeptide further comprises motifs 4-6 (SEQ ID NOs 61-63) or a fragment or variant thereof. The BBM polypeptide can further comprise motifs 8 and 9 (SEQ ID NOs: 69 and 70, respectively) or a fragment or variant thereof, and in some embodiments, motif 10 (SEQ ID NO: 68) or a variant or fragment thereof. In some of these embodiments, the BBM polypeptide also comprises at least one of motif 14 (set forth in SEQ ID NO: 71), motif 15 (set forth in SEQ ID NO: 72), and motif 19 (set forth in SEQ ID NO: 73), or variants or fragments thereof. The variant of a particular amino acid motif can be an amino acid sequence having at least about 40%, 50%, 60%, 70%, 75%, 80%, 85%, 90%, 95%, 96%, 97%, 98%, 99%, or greater sequence identity with the motif disclosed herein. Alternatively, variants of a particular amino acid motif can be an amino acid sequence that differs from the amino acid motif by 1, 2, 3, 4, 5, 6, 7, 8, 9, or 10 amino acids.

Non-limiting examples of babyboom polynucleotides and polypeptides that can be used in the methods and compositions include the Arabidopsis thaliana AtBBM (SEQ ID NOs: 74 and 75), Brassica napus BnBBM1 (SEQ ID NOs: 76 and 77), Brassica napus BnBBM2 (SEQ ID NOs: 78 and 79), Medicago truncatula MtBBM (SEQ ID NOs: 80 and 81), Glycine max GmBBM (SEQ ID NOs: 82 and 83), Vitis vinifera VvBBM (SEQ ID NOs: 84 and 85), Zea mays ZmBBM (SEQ ID NOs: 55 and 56 and genomic sequence set forth in SEQ ID NO: 57; or SEQ ID NOs: 58 and 59 and genomic sequence set forth in SEQ ID NO: 60) and ZmBBM2 (SEQ ID NOs: 101 and 102), Oryza sativa OsBBM (polynucleotide sequences set forth in SEQ ID NOs: 86 and 87; amino acid sequence set forth in SEQ ID NO: 89; and genomic sequence set forth in SEQ ID NO: 88), OsBBM1 (SEQ ID NOs: 90 and 91), OsBBM2 (SEQ ID NOs: 92 and 93), and OsBBM3 (SEQ ID NOs: 94 and 95), Sorghum bicolor SbBBM (SEQ ID NOs: 96 and 97 and genomic sequence set forth in SEQ ID NO: 98) and SbBBM2 (SEQ ID NOs: 99 and 100) or active fragments or variants thereof. In particular embodiments, the cell proliferation factor is a maize BBM polypeptide (SEQ ID NO: 56, 59, or 102) or a variant or fragment thereof, or is encoded by a maize BBM polynucleotide (SEQ ID NO: 55, 57, 121, 116, or 101) or a variant or fragment thereof.

Thus, in some embodiments, a polynucleotide encoding a cell proliferation factor has a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth in SEQ ID NO: 82, 96, 84, 80, 55, 101, 86, 90, 92, 94, 74, 76, 78, 99, 57, 60, 88, 87, 58, or 98 or the cell proliferation factor has an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 83, 97, 85, 81, 56, 102, 89, 91, 93, 95, 75, 77, 79, 59, or 100. In some of these embodiments, the cell proliferation factor has at least one of motifs 7 and 10 (SW ID NO: 67 and 68, respectively) or a variant or fragment thereof at the corresponding amino acid residue positions in the babyboom polypeptide. In other embodiments, the cell proliferation factor further comprises at least one of motif 14 (set forth in SEQ ID NO: 71), motif 15 (set forth in SEQ ID NO: 72), and motif 19 (set forth in SEQ ID NO: 73) or a variant or fragment thereof at the corresponding amino acid residue positions in the babyboom polypeptide.

In other embodiments, other cell proliferation factors, such as, Lec1, Kn1 family, WUSCHEL (e.g., WUS1, the polynucleotide and amino acid sequence of which is set forth in SEQ ID NO: 103 and 104; WUS2, the polynucleotide and amino acid sequence of which is set forth in SEQ ID NO: 105 and 106; WUS2 alt, the polynucleotide and amino acid sequence of which is set forth in SEQ ID NO: 107 and 108; WUS3, the polynucleotide and amino acid sequence of which is set forth in SEQ ID NO: 109 and 110), Zwille, and Aintegumeta (ANT), may be used alone, or in combination with a babyboom polypeptide or other cell proliferation factor. See, for example, U.S. Application Publication No. 2003/0135889, International Application Publication No. WO 03/001902, and U.S. Pat. No. 6,512,165, each of which is herein incorporated by reference.

In some embodiments, the polynucleotide construct comprises a polynucleotide encoding a Wuschel polypeptide (see International Application Publication No. WO 01/23575 and U.S. Pat. No. 7,256,322, each of which are herein incorporated by reference in its entirety). In certain embodiments, the polynucleotide encoding the Wuschel polypeptide has the sequence set forth in SEQ ID NO: 103, 105, 107, or 109 (WUS1, WUS2, WUS2 alt, or WUS3, respectively) or an active variant or fragment thereof. In particular embodiments, the Wuschel polypeptide has the sequence set forth in SEQ ID NO: 104, 106, 108, or 110 (WUS1, WUS2, WUS2 alt, or WUS3, respectively) or an active variant or fragment thereof. In some of these embodiments, the polynucleotide encoding a Wuschel polypeptide is operably linked to a promoter active in the plant, including but not limited to the maize In2-2 promoter or a nopaline synthase promoter.

When multiple cell proliferation factors are used, or when a babyboom polypeptide is used along with any of the abovementioned polypeptides, the polynucleotides encoding each of the factors can be present on the same expression cassette or on separate expression cassettes. When two or more factors are coded for by separate expression cassettes, the expression cassettes can be provided to the plant simultaneously or sequentially. In some embodiments, the polynucleotide construct comprises a polynucleotide encoding a babyboom polypeptide and a polynucleotide encoding a Wuschel polypeptide within the excision cassette such that the cell proliferation factors enhance the transformation frequency of the polynucleotide construct, but are subsequently excised upon desiccation of the transformed plant cell/tissue.

In some embodiments, herbicide tolerance polynucleotides can serve as a selectable marker for the identification of plants or plant parts that further comprise a polynucleotide of interest. Thus, in certain embodiments, the presently disclosed polynucleotide constructs can further comprise a polynucleotide of interest. In some embodiments, the polynucleotide of interest is operably linked to a promoter that is active in a plant cell. The promoter that is operably linked to the polynucleotide of interest can be a constitutive promoter, an inducible promoter, or a tissue-preferred promoter.

In certain embodiments, the polynucleotide of interest, and optionally the operably linked promoter, are located outside of the excision cassette on the polynucleotide construct. In other embodiments, the polynucleotide of interest and optionally its operably linked promoter are located within the excision cassette and the herbicide tolerance polynucleotide serves as a selectable marker to identify those plants or plant parts from which the polynucleotide of interest has been excised.

The polynucleotide of interest may impart various changes in the organism, particularly plants, including, but not limited to, modification of the fatty acid composition in the plant, altering the amino acid content of the plant, altering pathogen resistance, and the like. These results can be achieved by providing expression of heterologous products, increased expression of endogenous products in plants, or suppressed expression of endogenous products in plants.

General categories of polynucleotides of interest include, for example, those genes involved in information, such as zinc fingers, those involved in communication, such as kinases, those involved in biosynthetic pathways, and those involved in housekeeping, such as heat shock proteins. More specific categories of transgenes, for example, include sequences encoding important traits for agronomics, insect resistance, disease resistance, sterility, grain characteristics, oil, starch, carbohydrate, phytate, protein, nutrient, metabolism, digestability, kernel size, sucrose loading, and commercial products.

Traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Protein modifications to alter amino acid levels are described in U.S. Pat. Nos. 5,703,049, 5,885,801, 5,885,802, and 5,990,389 and WO 98/20122, herein incorporated by reference.

Insect resistance genes may encode resistance to pests such as rootworm, cutworm, European Corn Borer, and the like. Such genes include, for example, Bacillus thuringiensis toxic protein genes (U.S. Pat. Nos. 5,366,892; 5,747,450; 5,737,514; 5,723,756; 5,593,881; and Geiser et al. (1986) Gene 48:109); lectins (Van Damme et al. (1994) Plant Mol. Biol. 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

Commercial traits can also be encoded on a gene or genes that could, for example increase starch for ethanol production, or provide expression of proteins.

Although the herbicide tolerance polynucleotide can serve as a selectable marker to aid in the identification of transgenic plants that comprise a polynucleotide of interest or lack a polynucleotide of interest, an additional selectable marker may be present in the excision cassette of the presently disclosed polynucleotide constructs that aids in the selection of transgenic plants or plant parts at an earlier point in development when most herbicide selection systems are less efficient. In general, the selectable marker that is present within the excision cassette is one that allows for efficient selection in early stages of plant development and production (e.g., during the tissue proliferation stage of transgenic plant production). For example, the expression of a fluorescent protein can be used to select plants or plant parts that comprise a presently disclosed polynucleotide construct during or prior to tissue proliferation. Proliferating the tissue to a certain mass is generally necessary before regeneration of the tissue into a plant. The expression of the site-specific recombinase is then induced before herbicide selection, which in general, occurs during or after the regeneration of the provided cells or tissues into plants.

“Regenerating” or “regeneration” of a plant cell is the process of growing a plant from the plant cell (e.g., plant protoplast, callus or explant).

Marker genes that can be present within the excision cassette include polynucleotides encoding products that provide resistance against otherwise toxic compounds (e.g. antibiotic resistance) such as those encoding neomycin phosphotransferase II (NEO or nptII) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D), including but not limited to, the selectable marker gene phosphinothricin acetyl transferase (PAT) (Wohlleben et al. (1988) Gene 70:25-37), which confers resistance to the herbicide Bialaphos. In certain embodiments, the selectable marker that is present within the excision cassette is not a herbicide tolerance polynucleotide.

As used herein, “antibiotic resistance polypeptide” refers to a polypeptide that confers resistance or tolerance to an antibiotic compound to a host cell comprising or secreting the polypeptide.

Additional selectable marker-encoding polynucleotides include those that encode products that can be readily identified, including but not limited to phenotypic markers such as β-galactosidase, and visual markers, such as fluorescent proteins. As used herein, a “fluorescent protein” or “fluorescent polypeptide” refers to a polypeptide that is capable of absorbing radiation (e.g., light at a wavelength in the visible spectrum) at one wavelength and emitting radiation as light at a different wavelength. Non-limiting examples of fluorescent protein include green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), red fluorescent protein, and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The presently provided methods and compositions can also utilize metabolic enzymes as selectable markers. The term “metabolic enzymes” as it relates to selectable markers refer to enzymes that confer a selectable metabolic advantage to cells. Cells expressing the metabolic enzyme are then positively selected for the ability to metabolize and utilize a particular chemical compound that cannot otherwise be metabolized or utilized by other cells not comprising the enzyme. Non-limiting examples of metabolic enzymes for use as selectable markers include D-amino oxidase (encoded by the doa1 gene), which catalyzes the oxidative deamination of various D-amino acids (see, for example, Erikson et al. (2004) Nature Biotechnology 22:455-458, which is herein incorporated by reference in its entirety); cyanamide hydratase (encoded by the cah gene), which converts cyanamide into urea as a fertilizer source (see, for example, U.S. Pat. No. 6,268,547, which is herein incorporated by reference in its entirety); and phosphomannose isomerase (encoded by the pmi gene), which catalyzes the reversible inter-conversion of mannose-6-phosphate and fructose-6-phosphate, allowing plant cells to utilize mannose as a carbon source (see, for example, Joersbo et al. (1998) Molecular Breeding 4:11-117, which is herein incorporated by reference in its entirety).

In some embodiments, the excision cassette comprises more than one selectable marker-coding polynucleotide. In some of these embodiments, the excision cassette comprises both a visual marker and an antibiotic resistance or herbicidal resistance selectable marker. In some of these embodiments, the excision cassette comprises a maize optimized PAT-coding polynucleotide (such as the sequence set forth in SEQ ID NO: 54) or a polynucleotide encoding neomycin phosphotransferase II (NEO or nptII), and a polynucleotide encoding a fluorescent protein, such as yellow fluorescent protein.

The selectable marker-encoding polynucleotide within the excision cassette is operably linked to a promoter that is active in a plant cell. This promoter can be present within or outside of the excision cassette. In some of the embodiments wherein the promoter that is operably linked to the selectable marker-encoding polynucleotide is outside of the excision cassette, this same promoter will become operably linked to the herbicide tolerance polynucleotide after excision of the excision cassette.

In certain embodiments, the promoter that is operably linked to the selectable marker-encoding polynucleotide present within the excision cassette is a constitutive promoter such that the selectable marker will be constitutively expressed in the plant or plant part until excision of the excision cassette. In some of these embodiments, the constitutive promoter is a maize ubiquitin promoter, which in some embodiments comprises the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113).

During the selection of the plant or plant part that expresses the selectable marker that is found within the excision cassette, the plant or plant part can be cultured in the presence of a selection agent. As used herein, a “selection agent” refers to a compound that when contacted with a plant or plant part allows for the identification of a plant or plant part expressing a selectable marker, either positively or negatively. For example, a selection agent for an antibiotic resistance polynucleotide is the antibiotic to which the polynucleotide confers resistance. As a further non-limiting example, a selection agent for a metabolizing enzyme selectable marker is the compound that can only be metabolized and utilized by the cell that expresses the selectable marker.

In particular embodiments wherein the polynucleotide construct is designed for transformation of maize, the polynucleotide construct comprises, outside of the excision cassette, the expression cassettes for a GLYAT polypeptide and an ALS-inhibitor tolerance polypeptide as present in the T-DNA region of plasmid PHP24279 described in U.S. Pat. No. 7,928,296, which is herein incorporated by reference in its entirety. In these embodiments, the polynucleotide construct comprises the glyat4621 gene that was derived from the soil bacterium Bacillus licheniformis and was synthesized by a gene shuffling process to optimize the acetyltransferase activity of the GLYAT4621 enzyme (Castle et al. (2004) Science 304:1151-1154). The polynucleotide construct further comprises a ZM-HRA expression cassette comprising a modified maize acetolactate synthase gene, zm-hra (Zea mays-highly resistant allele), encoding the ZM-HRA protein, which confers tolerance to a range of ALS-inhibiting herbicides, such as sulfonylureas. In these embodiments, the glyat4621 gene cassette and the zm-hra gene cassette are in reverse orientation. The expression of the glyat4621 gene is controlled by the ubiquitin regulatory region from maize (ubiZM1 promoter (SEQ ID NO: 111), 5′UTR (SEQ ID NO: 112), and intron (SEQ ID NO: 112) (Christensen et al. (1992)) and the pinII terminator (An et al. (1989) Plant Cell 1:115-122). The expression of the zm-hra gene is controlled by the native maize acetolactate synthase promoter (zm-als promoter) (Fang et al. (2000)). The terminator for the zm-hra gene is the 3′ terminator sequence from the proteinase inhibitor II gene of Solanum tuberosum (pinII terminator). Upstream of both cassettes are three copies of the enhancer region from the cauliflower mosaic virus (CaMV 35S enhancer, U.S. application Ser. No. 11/508,045, herein incorporated by reference) providing expression enhancement to both cassettes.

In certain embodiments wherein the polynucleotide construct is designed for transformation of soybean (Glycine max), the polynucleotide construct comprises, outside of the excision cassette, the expression cassettes for a GLYAT polypeptide and an ALS-inhibitor tolerance polypeptide as present in the Not I-Asc I fragment of plasmid PHP20163 described in U.S. Pat. No. 7,622,641, which is herein incorporated by reference in its entirety. In these embodiments, the polynucleotide construct comprises the glyphosate acetyltransferase (glyat) gene derived from Bacillus licheniformis and a modified version of the soybean acetolactate synthase gene (zm-hra). The glyat gene was functionally improved by a gene shuffling process to optimize the kinetics of glyphosate acetyltransferase (GLYAT) activity for acetylating the herbicide glyphosate. The glyat gene is under the control of the SCP1 promoter and Tobacco Mosaic Virus (TMV) omega 5′ UTR translational enhancer element and the proteinase inhibitor II (pinII) terminator from Solanum tuberosum. The zm-hra gene is under the control of the S-adenosyl-L-methionine synthetase (SAMS) promoter and the acetolactate synthase (gm-als) terminator, both from soybean.

In other embodiments wherein the polynucleotide construct is designed for transformation of Brassica, the polynucleotide construct comprises the expression cassette for a GLYAT polypeptide as present in the plasmid PHP28181 described in U.S. Appl. Publ. No. 2012/0131692, which is herein incorporated by reference in its entirety. In these embodiments, the polynucleotide construct comprises the glyat4621 gene, which was derived from the soil bacterium Bacillus licheniformis and was synthesized by a gene shuffling process to optimize the acetyltransferase activity of the GLYAT4621 enzyme (Castle, et al., (2004) Science 304:1151-1154). The expression of the glyat4621 gene is controlled by the UBQ10 regulatory region from Arabidopsis and the pinII terminator. In some of these embodiments, the polynucleotide construct further comprises an expression cassette for an ALS inhibitor tolerance polypeptide.

The presently disclosed compositions and methods can utilize fragments or variants of known polynucleotide or polypeptide sequences. By “fragment” is intended a portion of the polynucleotide or a portion of an amino acid sequence and hence protein encoded thereby. Fragments of a polynucleotide may retain the biological activity of the native polynucleotide and, for example, have promoter activity (promoter fragments), or are capable of stimulating proliferation, inducing embryogenesis, modifying the regenerative capacity of a plant (cell proliferation factor fragments), are capable of conferring herbicide tolerance (herbicide tolerance polypeptide fragments) or catalyzing site-specific recombination (site-specific recombinase fragments). In those embodiments wherein the polynucleotide encodes a polypeptide, fragments of the polynucleotide may encode protein fragments that retain the biological activity of the native protein. Alternatively, fragments of a polynucleotide that are useful as hybridization probes generally do not retain biological activity or encode fragment proteins that retain biological activity. Thus, fragments of a nucleotide sequence may range from at least about 20, 50, 100, 150, 200, 250, 300, 400, 500 nucleotides, or greater.

A fragment of a polynucleotide that encodes a biologically active portion of a cell proliferation factor, for example, will encode at least 15, 25, 30, 50, 100, 150, 200, 250, 300, 400, 500 contiguous amino acids, or up to the total number of amino acids present in the full-length cell proliferation factor. Fragments of a coding polynucleotide that are useful as hybridization probes or PCR primers generally need not encode a biologically active portion of a polypeptide.

“Variants” is intended to mean substantially similar sequences. For polynucleotides, a variant comprises a polynucleotide having deletions at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides encoding polypeptides conservative variants include those sequences that, because of the degeneracy of the genetic code, encode the amino acid sequence the polypeptide (e.g., cell proliferation factor). Naturally occurring variants such as these can be identified with the use of well-known molecular biology techniques, such as, for example, with polymerase chain reaction (PCR) and hybridization techniques. Variant polynucleotides also include synthetically derived polynucleotides, such as those generated, for example, by using site-directed mutagenesis. Generally, variants of a particular will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters.

Variants of a particular polynucleotide that encodes a polypeptide can also be evaluated by comparison of the percent sequence identity between the polypeptide encoded by a variant polynucleotide and the polypeptide encoded by the particular polynucleotide. Percent sequence identity between any two polypeptides can be calculated using sequence alignment programs and parameters. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion of one or more amino acids at the N-terminal and/or C-terminal end of the native protein; deletion and/or addition of one or more amino acids at one or more internal sites in the native protein; and/or substitution of one or more amino acids at one or more sites in the native protein. Variant proteins retain the desired biological activity of the native protein. For example, variant cell proliferation factors stimulate proliferation and variant babyboom polypeptides are capable of stimulating proliferation, inducing embryogenesis, modifying the regenerative capacity of a plant, increasing the transformation efficiency in a plant, increasing or maintaining the yield in a plant under abiotic stress, producing asexually derived embryos in a plant, and/or enhancing rates of targeted polynucleotide modification. Such variants may result from, for example, genetic polymorphism or from human manipulation. Biologically active variants of a native protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters. A biologically active variant of a native protein may differ from that protein by as few as 1-15 amino acid residues, as few as 1-10, such as 6-10, as few as 5, as few as 4, 3, 2, or even 1 amino acid residue.

Where appropriate, the coding polynucleotides may be optimized for increased expression in the transformed plant. That is, the coding polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the babyboom polynucleotide. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire coding polynucleotide, or one or more portions thereof, may be used as a probe capable of specifically hybridizing to a corresponding coding polynucleotide and messenger RNAs. To achieve specific hybridization under a variety of conditions, such probes include sequences that are unique among the particular family of coding polynucleotide sequences and are optimally at least about 10 nucleotides in length, and most optimally at least about 20 nucleotides in length. Such probes may be used to amplify corresponding coding polynucleotides from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH.

However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The presently disclosed polynucleotide constructs can be introduced into a host cell. By “host cell” is meant a cell, which comprises a heterologous nucleic acid sequence. Host cells may be prokaryotic cells such as E. coli, or eukaryotic cells such as yeast, insect, amphibian, or mammalian cells. In some examples, host cells are monocotyledonous or dicotyledonous plant cells. In particular embodiments, the monocotyledonous host cell is a sugarcane host cell.

An intermediate host cell may be used, for example, to increase the copy number of the cloning vector and/or to mediate transformation of a different host cell. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein are available using Bacillus sp. and Salmonella (Palva et al. (1983) Gene 22:229-235); Mosbach et al. (1983) Nature 302:543-545).

Methods are provided for regulating the expression of a herbicide tolerance polynucleotide, wherein a host cell is provided that comprises a presently disclosed polynucleotide construct and the expression of the site-specific recombinase is induced, thereby excising the excision cassette and allowing for the operable linkage of the herbicide tolerance polynucleotide and its promoter and the expression of the herbicide tolerance polynucleotide.

Such methods allow for the delay of the expression of a herbicide tolerance polynucleotide until a point in development at which herbicide selection is more effective.

Thus, methods are further provided for selecting a herbicide tolerant plant cell, wherein a population of plant cells are provided, wherein at least one plant cell within the population comprises a presently disclosed polynucleotide construct, inducing the expression of the recombinase, and contacting the population of cells with a herbicide to which the herbicide tolerant polypeptide confers tolerance in order to select for the herbicide tolerant plant cell.

As used herein, the term “population of plant cells” may refer to any one of the following: a grouping of individual plant cells; a grouping of plant cells present within a single tissue, plant or plant part; a population of plants; a population of plant tissues either from the same plant or different plants; a population of seeds either from the same plant or different plants; or a population of plant parts either from the same plant or different plants. The provided population of plant cells, plant tissues, plants, or plant parts may be contacted with the herbicide. Alternatively, the provided population of plant cells may be cultured into a population of plant tissues or a population of plants, which is then exposed to the herbicide. Likewise, a provided population of plant seeds may be planted to produce a population of plants, which is then exposed to the herbicide.

In some embodiments, the provided population of plant cells is cultured into a population of plant tissues or plants prior to, during, or after the induction step, and the population of plant tissues or plants is then contacted with the herbicide. In some of these embodiments, the population of plant tissues is contacted with the herbicide during the regeneration of the tissues into plants or the population of plants that were regenerated from the population of plant tissues is contacted with the herbicide.

In certain embodiments, the provided population of plant cells is a population of immature or mature seeds. In some of these embodiments, the provided population of seeds is planted prior to, during, or after the induction step to produce a population of plants, and the population of plants are contacted with the herbicide. In those embodiments wherein the provided population of plant cells is a population of immature seeds and the inducible promoter that regulates the expression of the site-specific recombinase is a drought-inducible promoter, the drought-inducible promoter is activated in response to the natural desiccation that occurs during the maturation of the immature seed into a mature seed.

In other embodiments, the provided population of plant cells is a population of plant tissues and these plant tissues are cultured into a population of plants prior to, during, or after the induction step and the population of plants are then contacted with the herbicide.

In yet other embodiments, the provided population of plant cells is a population of plants.

In some embodiments, the provision of a plant or plant part comprising a presently disclosed polynucleotide construct comprises introducing the polynucleotide construct into the plant or plant part.

“Introducing” is intended to mean presenting to the organism, such as a plant, or the cell the polynucleotide or polypeptide in such a manner that the sequence gains access to the interior of a cell of the organism or to the cell itself. The methods and compositions do not depend on a particular method for introducing a sequence into an organism or cell, only that the polynucleotide or polypeptide gains access to the interior of at least one cell of the organism. Methods for introducing polynucleotides or polypeptides into plants or plant parts are known in the art including, but not limited to, stable transformation methods, transient transformation methods, and virus-mediated methods.

“Stable transformation” is intended to mean that the nucleotide construct introduced into a plant integrates into a genome of the plant and is capable of being inherited by the progeny thereof “Transient transformation” is intended to mean that a polynucleotide is introduced into the plant and does not integrate into a genome of the plant or a polypeptide is introduced into a plant.

Protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski et al. (1984) EMBO J. 3:2717-2722), and ballistic particle acceleration (see, for example, U.S. Pat. No. 4,945,050; U.S. Pat. No. 5,879,918; U.S. Pat. No. 5,886,244; and, U.S. Pat. No. 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. (1990) Biotechnology 8:833-839 (maize); Hooykaas-Van Slogteren et al. (1984) Nature 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Rep 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Rep 12:250-255 and Christou and Ford (1995) Annals of Botany 75:407-413 (rice); Osjoda et al. (1996) Nat Biotechnol 14:745-750 (maize via Agrobacterium tumefaciens); all of which are herein incorporated by reference.

In specific embodiments, the polynucleotide constructs can be provided to a plant using a variety of transient transformation methods. Such transient transformation methods include, but are not limited to, the introduction of the polynucleotide construct directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) J Cell Sci 107:775-784, all of which are herein incorporated by reference. Alternatively, the polynucleotide construct can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use of particles coated with polyethylimine (PEI; Sigma #P3143).

In other embodiments, the polynucleotide construct may be introduced into plants or plant parts by contacting plants or plant parts with a virus or viral nucleic acids. Generally, such methods involve incorporating a nucleotide construct within a viral DNA or RNA molecule. It is recognized that the proteins encoded by the various coding polynucleotides of the polynucleotide construct may be initially synthesized as part of a viral polyprotein, which later may be processed by proteolysis in vivo or in vitro to produce the desired recombinant protein. Further, it is recognized that promoters also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Other methods of introducing polynucleotides into a plant or plant part can be used, including plastid transformation methods, and the methods for introducing polynucleotides into tissues from seedlings or mature seeds.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide can be contained in a transfer cassette flanked by two non-recombinogenic recombination sites. The transfer cassette is introduced into a plant or plant part having stably incorporated into its genome a target site which is flanked by two non-recombinogenic recombination sites that correspond to the sites of the transfer cassette. An appropriate recombinase is provided and the transfer cassette is integrated at the target site. The polynucleotide construct is thereby integrated at a specific chromosomal position in the plant genome.

The cells that have been transformed may be grown into plants in accordance with conventional ways. See, for example, McCormick et al. (1986) Plant Cell Rep 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, transformed seed (also referred to as “transgenic seed”) having a nucleotide construct, for example, an expression cassette, stably incorporated into their genome is provided. Thus, compositions of the invention include plant cells, plant tissues, plant parts, and plants comprising the presently disclosed polynucleotide constructs. Likewise, the methods of the invention can be performed in plant cells, plant tissues, plant parts, and plants.

In certain embodiments the presently disclosed polynucleotide constructs can be stacked with any combination of polynucleotide sequences of interest in order to create plants with a desired trait. A trait, as used herein, refers to the phenotype derived from a particular sequence or groups of sequences. Plants that have various stacked combinations of traits can be created by any method including, but not limited to, cross-breeding plants by any conventional or TopCross methodology, or genetic transformation. If the sequences are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of a polynucleotide of interest. This may be combined with any combination of other suppression cassettes or overexpression cassettes to generate the desired combination of traits in the plant. It is further recognized that polynucleotide sequences can be stacked at a desired genomic location using a site-specific recombination system. See, for example, WO99/25821, WO99/25854, WO99/25840, WO99/25855, and WO99/25853, all of which are herein incorporated by reference.

Any plant species can be transformed, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum spp.), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Peryea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena), barley (Hordeum), Arabidopsis, switchgrass, vegetables, ornamentals, grasses, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). sugarcane (Saccharum spp.). In other embodiments, the plants are maize, rice, sorghum, barley, wheat, millet, oats, sugarcane, turfgrass, or switch grass. In specific embodiments, the plant is sugarcane.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In certain embodiments, the plant or plant part is a winter wheat plant or plant part. As used herein, “winter wheat” refers to wheat plants or plant parts that require an extended period of low temperatures to be able to flower. Non-limiting examples of winter wheat include Triticum aestivum and Triticum monococcum.

As used herein, the term “plant part” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides.

Methods are also provided for increasing transformation frequency, wherein a host cell is provided that comprises a presently disclosed polynucleotide construct comprising an excision cassette separating a polynucleotide encoding a herbicide tolerance polypeptide from its promoter, wherein the excision cassette comprises a polynucleotide encoding a site-specific recombinase that when expressed can excise the excision cassette. The population of plant cells comprising the polynucleotide construct is cultured in the absence of a herbicide to which the herbicide tolerance polypeptide confers herbicide resistance for a period of time sufficient for the population of plant cells to proliferate, followed by the induction of the expression of the site-specific recombinase, thereby excising the excision cassette and allowing for the operable linkage of the herbicide tolerance polynucleotide and its promoter and the expression of the herbicide tolerance polynucleotide allowing for the direct herbicide selection, thereby the transformation frequency is increased compared to a comparable plant cell not comprising the excision cassette and selected directly by herbicide selection. In some embodiments, the herbicide is glyphosate. In some embodiments, the induction comprises desiccating the population of plant cells. In some embodiments the induction comprises cold treatment.

By “period of time sufficient for the population cells to proliferate” is intended to mean that the population of cells has proliferated to a size and quality to produce transgenic events at an optimal level. The time period sufficient for the cells to proliferate may vary depending on the plant species, cultivar, explant and proliferation medium. In some embodiments, the population of plant cells is cultured in the absence of the herbicide to which the herbicide tolerance polypeptide confers herbicide resistance for about 1 hour to about 12 weeks, about 1 day to about 12 weeks, about 1 week to about 12 weeks, or about 1 week to 6 weeks, including but not limited to about 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, and 12 weeks. In other embodiments, the population of plant cells is cultured in the absence of the herbicide to which the herbicide tolerance polypeptide confers herbicide resistance for about 1 day to about 6 weeks, about 1 day to about 2 weeks, about 1 day to about 4 weeks, about 2 days to about 6 weeks, about 4 days to about 6 weeks, about 1 week to about 6 weeks, about 2 weeks to about 6 weeks, about 2 weeks to about 4 weeks, or about 2 weeks to about 3 weeks prior to excision.

“Transformation frequency” refers to the percentage of plant cells that are successfully transformed with a heterologous nucleic acid after performance of a transformation protocol on the cells to introduce the nucleic acid. In some embodiments, transformation further includes a selection protocol to select for those cells that are expressing one or more proteins encoded by a heterologous nucleic acid of interest. In some embodiments, transformation makes use of a “vector,” which is a nucleic acid molecule designed for transformation into a host cell.

An increased “transformation efficiency,” as used herein, refers to any improvement, such as an increase in transformation frequency, increased quality events frequency, labor saving, and/or decrease in ergonomic impact that impact overall efficiency of the transformation process by reducing the amount of resources required.

In general, upon use of the methods taught herein, transformation frequency is increased by at least about 3%, 5%, 7%, 8%, 9%, 10%, 11%, 12%, 13%, 14%, 15%, 20%, 30%, 40%, 50%, 60%, 70%, 80%, 90% or 100% or greater, or even 1-, 2-, 3-, 4-, 5-, 6-, 7-, 8-, 9-, 10-fold or more, than the transformation frequency relative to a control. The “control” provides a reference point for measuring changes in phenotype of the subject plant or plant cell, e.g., transformation frequency/efficiency, callus quality or transformation process time. The control may include, for example, plant cells transformed with a corresponding nucleic acid without the excision cassette.

In certain embodiments, the plant or plant part useful in the presently disclosed methods and compositions is recalcitrant. As used herein, a “recalcitrant plant” or “recalcitrant plant part” is a plant or plant part in which the average transformation frequency using typical transformation methods is relatively low, and typically less than about 1%, 2%, 3%, 4%, 5%, 6%, 7%, 8%, 9%, 10%, 11%, 12%, 13%, 14%, 15%, 16%, 17%, 18%, 19%, 20%, 25%, or 30%. The transformation of species, varieties or cultivars recalcitrant to transformation is time consuming, laborious, and inefficient compared to the transformation of non-recalcitrant varieties, with respect to one or more methods of transformation (e.g., Agrobacterium-mediated transformation). Non-limiting examples of species recalcitrant to Agrobacterium-mediated transformation include, but are not limited to, species of Lolium (rye grass), elite varieties of maize, cultivars of sugarcane, species of rice (especially Indica), and various turf grass species. In some embodiments, the recalcitrant plant or plant part is unable to be transformed in the absence of a cell proliferation factor. In certain embodiments, the recalcitrant plant or plant part is an elite maize inbred or a cell or tissue thereof. In other embodiments, the recalcitrant plant or plant part is the sugarcane cultivar CP96-1252, CP01-1372, CPCL97-2730, HoCP85-845, or CP89-2143 or a cell or tissue thereof.

In some embodiments of the present methods the recalcitrant plant part is an explant from a model or recalcitrant inbred or cultivar. In some embodiments of the present methods and compositions, the explant is from a recalcitrant inbred having a type I callus genotype. In some embodiments of the present methods and compositions, the explant is from a recalcitrant maize inbred having a type I callus genotype. Callus in grasses can be classified as type I or type II, based upon color, texture, regeneration system, and the amount of time required for callus initiation. The morphology of callus has been reported and described in the agronomically important monocot crops such as maize (Armstrong et al. (1985) Planta 164:207-214; Assam (2001) Arab J Biotechnol 4:247 256; Frame et al. (2000) In Vitro Cell Dev Biol-Plant 36:21-29; Lu et al. (1982) L. Theor Appl Genet 62:109-112; McCain et al. (1988) Bot Gazette 149:16-20; Songstad et al. (1992) Am J Bat 79:761-764; Welter et al. (1995) Plant Cell Rep 14:725-729; each of which is herein incorporated by reference in its entirety), rice (Chen et al. (1985) Plant Cell Tissue Organ Cult 4:51-51; Nakamura et al. (1989) Japan J Crop Sci 58:395-403; Rueb et al. (1994) Plant Cell Tissue Organ Cult 36:259-264; each of which is herein incorporated by reference in its entirety), sorghum (Jeoung et al. (2002) Hereditas 137:20-28; which is herein incorporated by reference in its entirety), sugarcane (Guiderdoni et al. (1988) Plant Cell Tissue Organ Cult 14:71-88; which is herein incorporated by reference in its entirety), wheat (Redway et al. (1990) Theor Appl Genet 79:609-617; which is herein incorporated by reference in its entirety), and various nonfood grasses. Type I callus is the typical and most prevalent callus formed in monocot species. It is characterized by compact form, slow-growth, white to light yellow in color, and highly organized. This callus is composed almost entirely of cytoplasmic meristematic cells that lack large vacuoles. In maize, type I callus can only be maintained for a few months and cannot be used in suspension cultures; whereas, type II callus can be maintained in culture for extended periods of time and is able to form cell suspensions. Type II callus derived from maize has been described as soft, friable, rapidly growing and exceedingly regenerative but is typically formed at lower frequencies than type I callus. Embryogenic suspension cells can be initiated from type II callus, which few maize lines can form. Although the ability to form type II callus can be backcrossed into agronomically important maize lines, in practice this is time consuming and difficult. Moreover, even for those lines that can form type II callus, the method requires a great deal of time and labor and is, therefore, impractical. Normally, recalcitrant inbred or cultivar genotypes that produce type I callus have low transformation frequencies. Typically with maize type I inbreds large numbers of embryos or other explants must be screened to identify sufficient quantities of events, which is expensive and labor intensive.

It is to be noted that the term “a” or “an” entity refers to one or more of that entity; for example, “a polynucleotide” is understood to represent one or more polynucleotides. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

The following examples are offered by way of illustration and not by way of limitation.

### EXPERIMENTAL

### Example 1

### Glyphosate Selection of Transformed Maize Inbred PHR03

Immature embryos from maize inbred PHR03 were harvested 9-13 days post-pollination with embryo sizes ranging from 0.8-2.5 mm length and were co-cultivated with Agrobacterium strain LBA4404 containing the vector PHP29204 or Agrobacterium strain LBA4404 containing the vector PHP32269 on PHI-T medium for 2-4 days in dark conditions. PHP29204:Ubi:DsRed+Ubi:GAT4602. PHP32269:Ubi:PMI+Ubi:MOPAT::YFP. Ubi refers to the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113). The tissues were then transferred to DBC3 medium without selection for one week, and then to DBC3 medium with 0.25 mM or 0.5 mM glyphosate for 3 weeks, and then DBC3 medium with 0.5 mM glyphosate for another 3-4 weeks. The embryos were then transferred to PHI-RF maturation medium with 0.1 mM glyphosate for 2-3 weeks until shoots formed, at which point, the shoots were transferred to MSB medium in Phytatrays containing 100 mg/L cefotaxime for rooting. Plants with good roots were transferred to soil for further growth and a glyphosate spray test. For PMI selection using PHP32269, DBC3 medium containing 12.5 g/L mannose and 5 g/L maltose was used for selection. PHI-RF maturation medium without any selective agent or sugar modifications was used for regeneration.

PHI-T medium contains 0.1 μM copper in MS salts 4.3 mg/L, Nicotinic acid 0.5 mg/L, Pyridoxine HCl 0.5 mg/L, Thiamine HCl 1 mg/L, Myo-inositol 100 mg/L, 2,4-D 2 mg/L, Sucrose 20 g/L, Glucose 10 g/L, L-proline 700 mg/L, MES 0.5 g/L, Acetosyringone 100 μM, Ascorbic acid 10 mg/L and Agar 8.0 g/L.

PHI-RF is 4.3 g/L MS salts (GIBCO BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.49 μM cupric sulfate, 0.5 mg/L zeatin (Sigma Z-0164), 1 mg/L IAA, 26.4 μg/L ABA, thidiazuron 0.1 mg/L, 60 g/L sucrose, 100 mg/L cefotaxime, 8 g/L agar, pH 5.6.

TABLE 4
Transformation frequency of maize inbred PHR03 with
PHP29204 or PHP32269.

| | | | % | No. single | % Single |
|---|---|---|---|---|---|
| | No. of | No. of T₀ | Transfor- | copy | Copy |
| Vector | embryos | events | mation | events | Events |
| PHP29204 | 300 | 21 | 7 | 13 | 61.9 |
| PHP32269 | 90 | 36 | 40 | 16 | 44.4 |

The transformation frequency with PHP29204 with glyphosate selection was only 7% in the maize inbred PHR03. Overall, glyphosate selection did not provide for a clean selection, a lot of non-transformed tissues were growing, and the morphology of both transformed and non-transformed tissues was irregular.

### Example 2

### Agrobacterium-Mediated Sugarcane Transformation Using a Standard Test Vector without Developmental Genes

### Media for Plant Transformation:

Liquid DBC3(M5G) contains MS salts (4.3 g/L) plus maltose (30 g/L); glucose (5 g/L); thiamine-HCl (1 mg/mL); myo-inositol (0.25 g/L); N-Z-amine-A (casein hydrolysate) (1 g/L); proline (0.69 g/L); CuSO₄ (4.9 μM); 2,4-D (1.0 mg/L); BAP (0.5 mg/L); Adjust volume to 1 L with ddH2O; pH 5.8—Adjust pH with 1 M KOH; autoclave.

DBC3 contains MS salts (4.3 g/L) plus maltose (30 g/L); thiamine-HCl (1 mg/mL); myo-inositol (0.25 g/L); N-Z-amine-A (casein hydrolysate) (1 g/L); proline (0.69 g/L); CuSO₄ (4.9 μM); 2,4-D (1.0 mg/L); BAP (0.5 mg/L); Adjust volume to 1 L with ddH₂O; pH 5.8—Adjust pH with 1 M KOH; Phytagel (3.5 g/L); autoclave.

DBC6 contains MS salts (4.3 g/L) plus maltose (30 g/L); thiamine-HCl (1 mg/mL); myo-inositol (0.25 g/L); N-Z-amine-A (casein hydrolysate) (1 g/L); proline (0.69 g/L); CuSO₄ (4.9 μM); 2,4-D (0.5 mg/L); BAP (2.0 mg/L); Adjust volume to 1 L with ddH₂O; pH 5.8—Adjust pH with 1 M KOH; Phytagel (3.5 g/L); autoclave.

MSB contains MS salts and vitamins (4.43 g/L) plus sucrose (20 g/L); myo-inositol (1.0 g/L); indole-3-butyric acid (IBA, 0.5 mg/L); Adjust volume to 1 L with ddH₂O; pH 5.8—Adjust pH with 1 M KOH; Phytagel (3.5 g/L); autoclave.

### Preparation of Agrobacterium Suspension:

Agrobacterium tumefaciens harboring a binary vector from a −80° frozen aliquot was streaked out onto solid PHI-L or LB medium containing an appropriate antibiotic and cultured at 28° C. in the dark for 2-3 days. A single colony or multiple colonies were picked from the master plate and streaked onto a plate containing PHI-M medium and incubated at 28° C. in the dark for 1-2 days. Agrobacterium cells were collected from the solid medium using 5 mL 10 mM MgSO₄ medium (Agrobacterium infection medium) plus 100 μM acetosyringone. One mL of the suspension was transferred to a spectrophotometer tube and the OD₅₀₀ₙₘ of the suspension was adjusted to 0.35-0.40 at 550 nm using the same medium.

### Agrobacterium Infection and Co-Cultivation:

Good quality callus tissues induced from in vitro-cultured plantlets were collected in an empty Petri dish and exposed to air in the hood for about 30 minutes. Tissue that is younger than 2 months old is considered ideal for transformation. One mL Agrobacterium suspension was added to the Petri dish, the tissues were broken or chopped into small pieces, and an additional 1-3 mL Agrobacterium (AGL1) suspension was then added to cover all the tissues. The Petri dish was placed into a transparent polycarbonate desiccator container, and the container was covered and connected to an in-house vacuum system for 20 minutes. After infection, the Agrobacterium suspension was drawn off from the Petri dish and the tissues were transferred onto 2 layers of VWR 415 filter paper (7.5 cm diameter) of a new Petri dish and 0.7-2.0 mL liquid DBC3 (M5G) medium plus 100 μM acetosyringone was added for cocultivation depending on the amount of tissue collected. The top layer of filter paper containing the infected tissues was transferred to a fresh layer of filter paper of another new Petri dish. The infected tissues were incubated at 21° C. in the dark for 3 days.

### Selection and Plant Regeneration:

Callus tissues were transferred to first round selection DBC3 containing antibiotics (timentin and cefotaxime) and 3 mg/L bialaphos (Meiji Seika, Tokyo, Japan). Tissues were transferred to 2nd round selection DBC6 containing antibiotics and 3-5 mg/L bialaphos and subcultured for 3 weeks at 26-28° C. in dark or dim light conditions. At the 3rd round selection on DBC6 medium containing antibiotics and bialaphos, tissues were broken into smaller pieces and exposed to bright light conditions (30-150 μmol m⁻² sec⁻¹) for 2-3 weeks. Shoot-elongated tissues were broken into small pieces and transferred to MSB regeneration/rooting medium containing antibiotics and 3 mg/L bialaphos. Single plantlets were separated and transferred to soil.

Table 5 shows the results of transformation experiments using 7 U.S. sugarcane cultivars. CP89-2376 and CP88-1762 had >100% transformation frequency at the Tₒ plant level using a standard vector containing DsRED and PAT (or moPAT) while the remaining 5 cultivars, CP96-1252, CP01-1372, CPCL97-2730, HoCP85-845 and CP89-2143, were recalcitrant in transformation.

TABLE 5
Transformation Frequencies at T₀ Plant Level in 7 U.S.
Sugarcane Cultivars Using a Standard Test Vector.

| CP96- | CP01- | CP89- | CPCL97- | HoCP85- | CP89- | CP88- |
|---|---|---|---|---|---|---|
| 1252 | 1372 | 2376 | 2730 | 845 | 2143 | 1762 |
| n.t.* | n.t. | 75.0% | n.t. | n.t. | n.t. | n.t. |
| | | (6/8) | | | | |
| 0% (0/8) | 0% (0/8) | 100.0% | 0% (0/8) | n.t. | n.t. | n.t. |
| | | (8/8) | | | | |
| n.t. | n.t. | 87.5% | n.t. | n.t. | n.t. | n.t. |
| | | (7/8) | | | | |
| n.t. | n.t. | 150.0% | n.t. | 0% (0/8) | n.t. | n.t. |
| | | (12/8) | | | | |
| n.t. | n.t. | n.t. | n.t. | n.t. | 0% (0/8) | 62.5% |
| | | | | | | (5/8) |
| n.t. | n.t. | 100.0% | n.t. | n.t. | 0% (0/8) | 137.5% |
| | | (8/8) | | | | (11/8) |
| n.t. | n.t. | 187.5% | n.t. | n.t. | n.t. | 137.5% |
| | | (15/8) | | | | (11/8) |

Transformation Frequency = (# transgenic events/# explants infected with Agrobacterium) × 100%
*n.t.: not tested

### Confirmation of Transgenic Events:

The putative stable callus/green tissues/regenerating plants were identified based on the visible RFP marker gene expression. All of these putative transgenic callus tissues were transferred to medium for plant regeneration under standard regeneration conditions. The final confirmation of stable transformation frequency was determined based on molecular analysis such as PCR and Southern blot hybridization.

### Example 3

### Sugarcane Transformation Using a Developmental Gene (DevGene) Vector PHP35648 and Excision Test

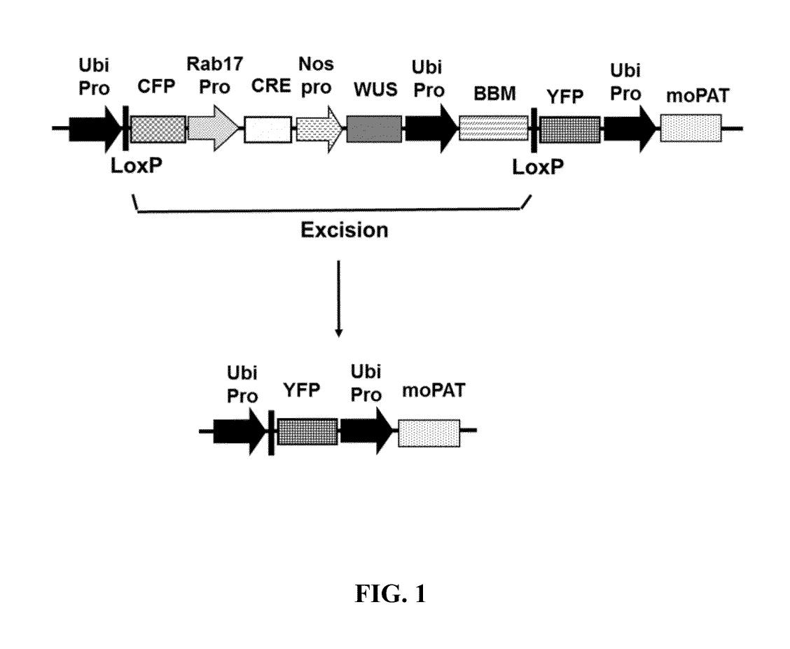

A DevGene binary vector (PHP35648, FIG. 1) with the BBM/WUS gene cassette was initially compared with a standard vector containing PAT or moPAT plus DsRED without the BBM/WUS gene cassette for transformation frequency using two Agrobacterium strains, AGL1 and LBA4404, in cultivar CP89-2376 and the recalcitrant cultivar CP01-1372 (Table 6). The DevGene binary vector contains Ubi::LoxP::CFP+Rab17Pro-attb1::Cre+Nos::ZmWUS2+Ubi::ZmBBM-LoxP::YFP+Ubi::MOPAT (FIG. 1); each gene cassette has a 3′ terminator. The Lox cassette containing CFP::Cre::WUS::BBM can be excised by Cre recombinase controlled by the Rab17 promoter. The PHP35648 vector was designed to demonstrate the excision efficiency of the excision cassette using visual markers. The PHP35648 excision cassette comprises the cyan fluorescent protein (CFP) controlled by the ubiquitin promoter (comprising the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113)), which is located outside of the loxP site flanking the excision cassette (see FIG. 1). Transformants comprising the excision cassette can be visually identified by the presence of the cyan fluorescent protein (CFP). When the excision cassette is excised, the yellow fluorescent protein (YFP) is expressed under the regulation of the ubiquitin promoter. Transformants lacking the excision cassette can be visually identified by the presence of the yellow fluorescent protein (YFP). The ratio of cyan fluorescent protein (CFP) to yellow fluorescent protein (YFP) can be used to demonstrate the excision efficiency. In PHP35648, the ubiquitin promoter controlling the expression of the moPAT gene product was included outside of the excision cassette as a positive selection to reduce the number of escapes.

Callus tissues of all 5 sugarcane cultivars were induced and maintained on DBC3 medium. Tissues were infected with Agrobacterium containing the DevGene binary vector PHP35648 in liquid 10 mM MgSO₄ plus 100 μM acetosyringone and then co-cultivated with liquid DBC3 (M5G) medium plus 100 μM acetosyringone on filter paper in Petri dishes at 21° C. in the dark. Three days after co-cultivation, the tissues were transferred to DBC3 containing 100 mg/L cefotaxime and 150 mg/L timentin for AGL1 and DBC3 containing 100 mg/L carbenicillin for LBA4404, and incubated at 26° C. (±1° C.) in the dark or dim light for 3-7 days. Afterwards, the tissues were transferred to the same media as the previous step plus 3 or 5 mg/L bialaphos. After 2 to 3 weeks, the tissues were transferred to 2nd round selection DBC6 containing antibiotics and 3-5 mg/L bialaphos. After two months from the initiation of the experiment, transformation frequency was calculated by the number of tissues showing CFP-expressing sectors divided by the number of explants infected by Agrobacterium. AGL1 was more efficient in transformation than LBA4404 in both CP89-2376 and CP01-1372 (Table 6, rows 1 and 2). There was also a genotype difference in transformation frequency; the CP89-2376 cultivar had a much higher transformation frequency than the recalcitrant cultivar CP01-1372 using either of the Agrobacterium strains.

AGL1 containing the DevGene binary vector PHP35648 was also used to test sugarcane germplasm screening in another set of four experiments (Table 6, rows 3-6) using 5 different cultivars (CP96-1252, CP01-1372, CP89-2376, CPCL97-2730 and HoCP85-845). Callus tissues of all 5 cultivars tested were induced and maintained on DBC3 medium and tissues were infected with AGL1 containing the developmental gene binary vector PHP35648. The use of developmental genes dramatically increased transformation frequency in all 5 cultivars tested. Transformation frequencies in the most amenable cultivar, CP89-2376, using a standard binary vector averaged 116.7% (56/48) (Table 6). In contrast, an average transformation frequency in CP89-2376 from the 5 experiments using the DevGene binary vector PHP35648 was >2,512.5% (>1,005 events/40 tissues infected) (see Table 6, rows 2-6). An increase in transformation frequency was also observed in the recalcitrant cultivars CP96-1252, CP01-1372, CPCL97-2730 and HoCP85-845; with transformation frequencies ranging from 62.5% to 1250.0% using AGL1 while no transgenic events were obtained using the standard vector without the BBM/WUS gene cassette from these cultivars (Table 6, row 7).

TABLE 6
Transformation Frequency in Sugarcane Using a BBM/WUS
Developmental Gene Vector PHP35648.

| Agrobacterium | | | | | | |
|---|---|---|---|---|---|---|
| | Binary | Sugarcane Cultivar | | | | |
| Strain | Vector | CP96-1252 | CP01-1372 | CP89-2376 | CPCL97-2730 | HoCP85-845 |
| AGL1 | DG^{a} | n.t.^{c} | 37.5% | n.t. | n.t. | n.t. |
| | | | (3/8) | | | |
| LBA4404 | DG | n.t. | 0% | n.t. | n.t. | n.t. |
| | | | (0/8) | | | |
| AGL1 | DG | n.t. | >1,250.0% | >6,250.0% | n.t. | n.t. |
| | | | (>100/8) | (>500/8) | | |
| LBA4404 | DG | n.t. | 12.5% | >1,500% | n.t. | n.t. |
| | | | (1/8) | (>120/8) | | |
| AGL1 | DG | n.t. | n.t. | 687.5% | n.t. | n.t. |
| | | | | (>55/8) | | |
| AGL1 | DG | n.t. | n.t. | >2,500% | 175.0% | n.t. |
| | | | | (>200/8) | (14/8) | |
| AGL1 | DG | 150.0% | 62.5% | >625.0% | 62.5% | n.t. |
| | | (12/8) | (5/8) | (>50/8) | (6/8) | |
| AGL1 | DG | n.t. | n.t. | >2,500% | n.t. | 187.5% |
| | | | | (>200/8) | | (15/8) |
| AGL1 | Std^{b} | 0% | 0% | 116.7% | 0% | 0% |
| | | (0/8) | (0/8) | (56/48) | (0/8) | (0/8) |

Each transformation treatment had 8 pieces of callus tissues 0.4-0.5 cm in size.
DG^{a}: developmental gene vector with BBM/WUS gene cassette
Std^{b}: standard vector without BBM/WUS gene cassette
n.t.^{c}.: not tested

### Excision of the LoxP Cassette by Dessication Monitored by Visual Markers

Transgenic callus tissues were desiccated on dry filter papers for one day to induce excision of the Lox cassette containing CFP::Cre::WUS::BBM by Cre recombinase driven by the Rab17 promoter (FIG. 1). Excision was monitored by observing YFP expression on desiccated transgenic callus events by the presence of the UBI:loxP:YFP junction formed as a result of excision (FIG. 1). Cre excision occurred on 83 of 87 transgenic events (95.4%) (Table 7). Plants from some transgenic events after excision were regenerated on MSB plus 1-3 mg/L bialaphos and antibiotics.

TABLE 7
Excision Efficiency of the BBM/WUS Gene Cassette in Transgenic
Sugarcane Events by Desiccation.

| Sugarcane | Agrobacterium | Binary | Excision Efficiency | |
|---|---|---|---|---|
| Cultivar | Strain | Vector | (%) | |
| CP89-2376 | AGL1 | DG^{a} | 93% | (40/43) |
| CP89-2376 | LBA4404 | DG | 100% | (25/25) |
| CP01-1372 | AGL1 | DG | 100% | (13/13) |
| CP01-1372 | LBA4404 | DG | 0% | (0/1) |
| CP89-2376 | AGL1 | DG | 100% | (5/5) |

| | | |
|---|---|---|
| Average | 95.4% | (83/87) |

DG^{a}: developmental gene (DevGene) vector PHP35648 with BBM/WUS gene cassette

### Example 4

### Sugarcane Excision Induction and Plant Regeneration from Transformed Callus/Green Tissue Events Generated Using a Developmental Gene (DevGene) Vector PHP54561

### Generation of Transgenic Events:

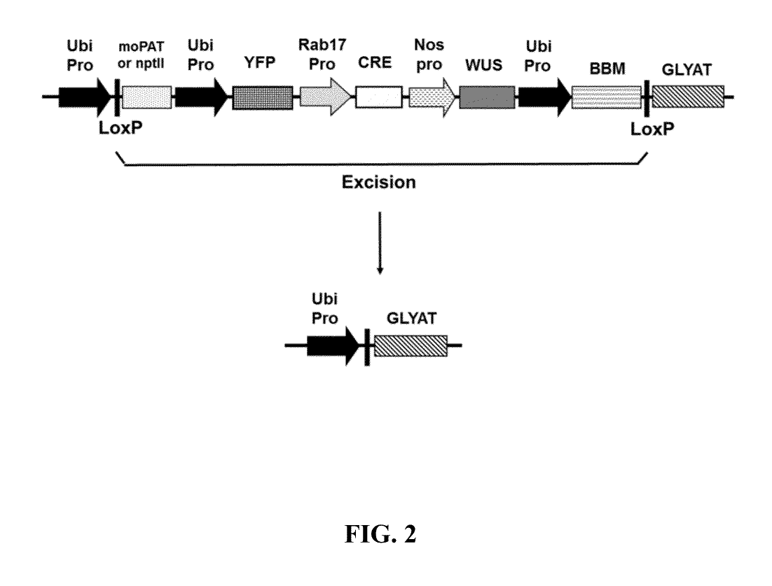

A new DevGene binary vector PHP54561 with the BBM/WUS gene cassette was designed as described in FIG. 2. The DevGene binary vector PHP54561 contains Ubi::LoxP-moPAT+Ubi:YFP+Rab17Pro-attb1:Cre+Nos:ZmWUS2+Ubi:ZmBBM-LoxP::GLYAT (FIG. 2); each gene cassette has a 3′ terminator. The Lox cassette containing moPAT+Ubi:YFP+Rab17Pro-attb1:Cre+Nos:ZmWUS2+Ubi:ZmBBM can be excised by Cre recombinase controlled by the Rab17 promoter. The PHP54561 excision cassette was designed to test the excision efficiency directly by glyphosate tolerance (see FIG. 2). The yellow florescent protein (YFP) was included in the PHP54561 excision cassette as a visual marker and moPAT as a selection marker prior to excision (see FIG. 2). Ubi refers to the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113).

Callus tissues of two U.S. sugarcane cultivars, CP88-1762, CP01-1372 and 1 Australian cultivar, KQ228, were induced and maintained on DBC3 or DBC6 medium. Tissues were infected with Agrobacterium containing the DevGene binary vector PHP54561 in liquid 10 mM MgSO₄ plus 100 μM acetosyringone and then co-cultivated with liquid DBC3 (M5G) medium plus 100 μM acetosyringone on the filter paper in Petri dishes at 21° C. in the dark. Three days after co-cultivation, the tissues of CP88-1762/CP01-1372 and KQ228 were transferred to DBC3 and DBC6 containing 100 mg/L cefotaxime and 150 mg/L timentin, respectively, and incubated at 26° C. (±1° C.) in the dark or dim light for 3-7 days. Afterwards, the tissues were transferred to the same media as the previous step plus 3 or 5 mg/L bialaphos. After 2 to 3 weeks, the tissues were transferred to 2nd round selection DBC6 containing antibiotics and 3-5 mg/L bialaphos. YFP-expressing sectors were transferred to the same medium for proliferation. After two months from the initiation of the experiment, transformation frequency was calculated by the number of tissues showing YFP-expressing sectors divided by the number of explants infected by Agrobacterium. Table 8 demonstrated transformation frequency at the T₀ tissue level in 3 sugarcane cultivars. CP88-1762, an amenable cultivar had 405% transformation. Two recalcitrant cultivars, CP01-1372 and KQ228 also had high transformation frequencies, 885% and 130%, respectively.

TABLE 8
Transformation Frequencies at the T₀ Tissue Level in Sugarcane
with Bialaphos Selection before Excision.

| Cultivar | Txn Frequency (%) | |
|---|---|---|
| CP01-1372* | 270% | (27/10) |
| CP01-1372* | 1500% | (150/10) |
| Total | 885% | (177/20) |
| CP88-1762 | 400% | (40/10) |
| CP88-1762 | 410% | (41/10) |
| Total | 405% | (81/20) |
| KQ228* | 10% | (1/10) |
| KQ228* | 250% | (25/10) |
| Total | 130% | (26/20) |

*CP01-1372 and KQ228 are recalcitrant commercial cultivars.

Excision of LoxP Cassette by Desiccation and Plant Regeneration with Glyphosate Selection:

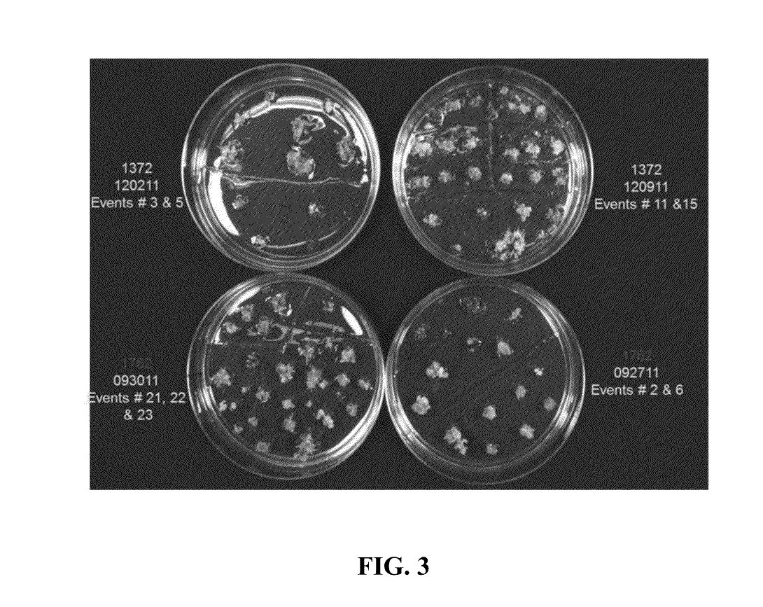

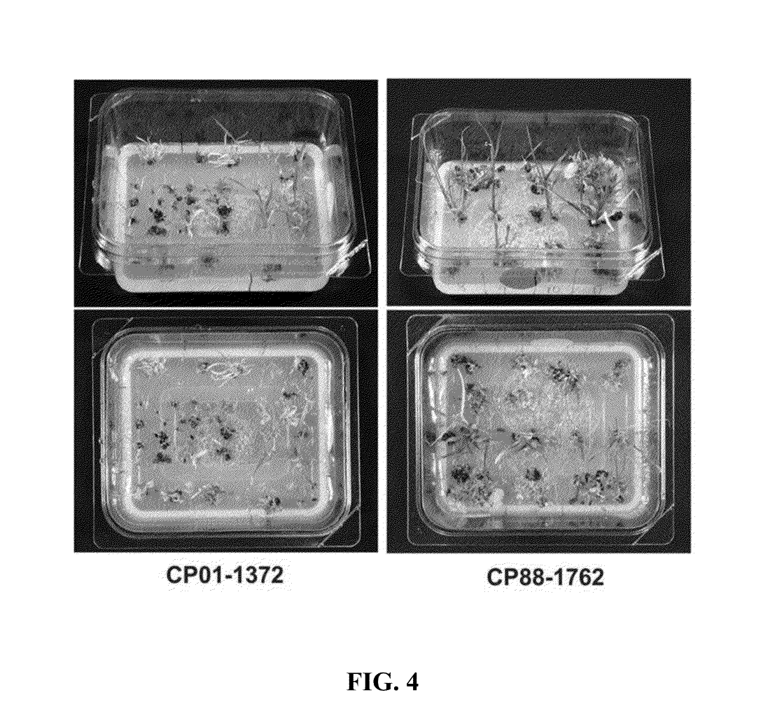

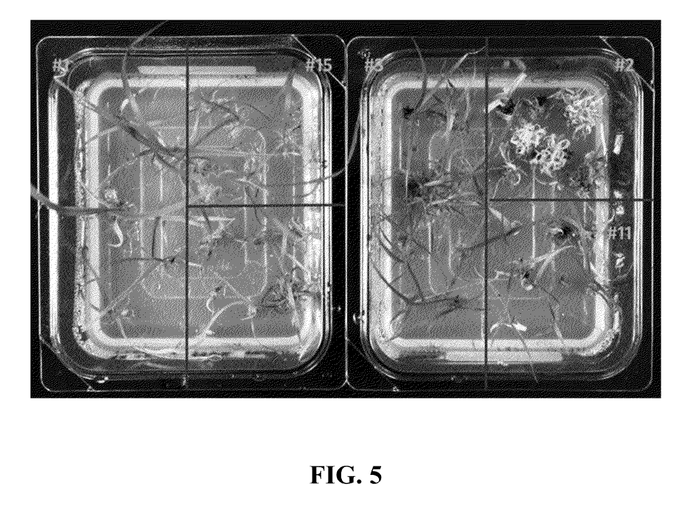

Transgenic tissues (0.3-0.5 mm in diameter) were transferred to an empty 60 mm×25 mm Petri dish containing a piece of sterilized glass filter paper (VWR Glass Microfibre filter, 691). The Petri dish was covered with a lid and placed in a container with a tight-seal cover. A Petri dish (or beaker) with ˜20 mL of sterilized water with the lid open was placed in the container. The container was kept in a dark culture room for 1-2.5 days at 28° C.; the desiccation period was dependent on the degree or size of tissues. After 1-2.5 days of desiccation treatment, the desiccated tissues were transferred to DBC6 proliferation medium with antibiotics and 100 μM glyphosate. The plates were kept in dim (10-50 μmol m⁻² sec⁻¹) to moderately bright light at 26-28° C. for 2-3 weeks (FIG. 3). If necessary, tissues were subcultured for another round on the same medium for another 2-3 weeks to get small green shoots; the plates was kept in a higher intensity of light at 26-28° C. Tissues with shoots were picked up and placed onto MSB regeneration/rooting medium containing antibiotics and 20-30 μM glyphosate in A175 Agar (PhytoTechnology Lab) as a gelling agent. Tissues were cultured under bright light conditions (50-200 μmmol m⁻² sec⁻¹) for 3-4 weeks at 26-28° C. When shoots were strong enough, single plantlets were separated and transferred to soil. In general, plants with complete excision exhibited a normal phenotype with greener and faster growth, while plantlets from tissues without excision of the developmental genes or having incomplete excision usually showed a stunted phenotype or bleached shoots, indicating susceptibility to glyphosate (FIGS. 4 and 5). Plants with a normal phenotype were transferred to soil for further growth, glyphosate spray test and molecular assay.

Table 9 shows LoxP cassette excision efficiency in transgenic events of 3 sugarcane cultivars, CP88-1762, CP01-1372 and KQ228, based on glyphosate resistance of the events. Excision efficiencies ranged from 32% to 68% in these 3 cultivars.

TABLE 9
Excision Efficiency with Glyphosate Selection of Transgenic Sugarcane Events
by Desiccation.

| | | | | # of events with | Excision Efficiency | |
|---|---|---|---|---|---|---|
| | Transformation | | # of events | green elongated | (# of events excised/ | |
| Cultivar | Frequency* | | desiccated | shoots on glyphosate | # of events desiccated) | |
| CP01-1372 | 270% | (27/10) | 12 | 8 | 66.7% | (8/12) |
| CP01-1372 | 1500% | (150/10) | 41 | 28 | 68.3% | (28/41) |
| Total | 885% | (177/20) | 53 | 36 | 67.9% | (36/53) |
| CP88-1762 | 400% | (40/10) | 15 | 6 | 40.0% | (6/15) |
| CP88-1762 | 410% | (41/10) | 38 | 20 | 52.6% | (20/38) |
| Total | 405% | (81/20) | 53 | 26 | 49.1% | (26/53) |
| KQ228 | 10% | (1/10) | 1 | 0 | 0% | (0/1) |
| KQ228 | 250% | (25/10) | 21 | 7 | 33.3% | (7/21) |
| Total | 130% | (26/20) | 22 | 7 | 31.8% | (7/22) |

*bialaphos selection before excision

### Glyphosate Resistance Confirmation by Glyphosate Spray Test:

T₀ plantlets were then moved to soil and spray tested with 4× glyphosate to confirm excision/glyphosate resistance. All 72 independent Tₒ events from 3 sugarcane cultivars (Table 9) showed strong glyphosate resistance while plants of 3 nontransgenic cultivars were completely killed by glyphosate spray. The final confirmation of stable transformation frequency is determined based on molecular analysis such as PCR and Southern blot hybridization.

### Example 5

### Corn Excision Induction and Plant Regeneration from Desiccated T₁ Immature Embryos

### Corn Transformation:

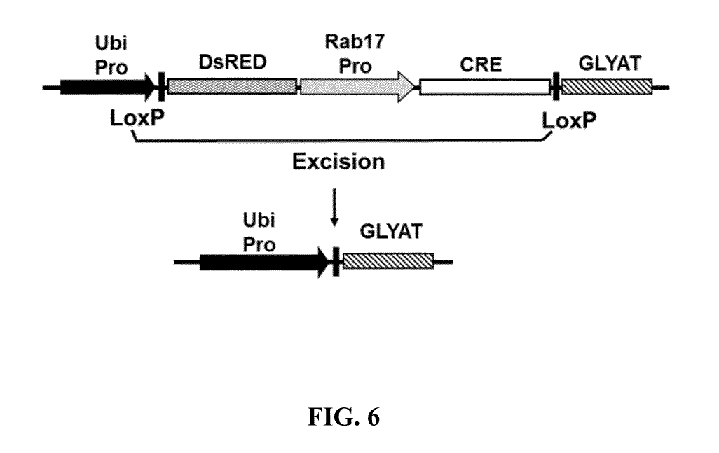

A corn elite inbred, PHR03 was transformed with Agrobacterium strain AGL1 containing the excision vector PHP54353. The PHP54353 vector contains Ubi::LoxP-Ds RED+Rab17-attB::CRE-LoxP::GLYAT (FIG. 6). The Lox cassette containing Ds RED+Rab17-attB::CRE can be excised by Cre recombinase controlled by the Rab17 promoter. The PHP54353 excision cassette was designed to demonstrate direct glyphosate selection. Ubi refers to the maize ubiquitin promoter (UBI1ZM PRO; SEQ ID NO: 111), the ubiquitin 5′ UTR (UBI1ZM 5UTR; SEQ ID NO: 112), and ubiquitin intron 1 (UBIZM INTRON1; SEQ ID NO: 113).

Immature embryos from maize inbred PHR03 were harvested 9-13 days post-pollination with embryo sizes ranging from 0.8-2.5 mm length and were co-cultivated with Agrobacterium strain AGL1 containing the excision vector PHP54353 on PHI-T medium for 3 days in dark conditions. These embryos were then transferred to DBC3 medium containing 100 mg/L cefotaxime in dim light conditions. RFP-expressing sectors were picked up and proliferated on the same medium. When the tissue proliferation period for each transgenic event was sufficient, tissues were moved to PHI-RF maturation medium. Regenerating shoots were transferred to MSB medium in Phytatrays containing 100 mg/L cefotaxime for rooting. Plants with good roots were transferred to soil for further growth, glyphosate spray test and molecular assay.

PHI-T medium contains 0.1 μM copper in MS salts 4.3 mg/L, Nicotinic acid 0.5 mg/L, Pyridoxine HCl 0.5 mg/L, Thiamine HCl 1 mg/L, Myo-inositol 100 mg/L, 2,4-D 2 mg/L, Sucrose 20 g/L, Glucose 10 g/L, L-proline 700 mg/L, MES 0.5 g/L, Acetosyringone 100 μM, Ascorbic acid 10 mg/L and Agar 8.0 g/L.

PHI-RF is 4.3 g/L MS salts (GIBCO BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.49 μM cupric sulfate, 0.5 mg/L zeatin (Sigma Z-0164), 1 mg/L IAA, 26.4 μg/L ABA, thidiazuron 0.1 mg/L, 60 g/L sucrose, 100 mg/L cefotaxime, 8 g/L agar, pH 5.6.

### Immature Embryo Isolation, Desiccation, Selection and Regeneration:

Sterilized immature embryos with 2.0-3.5 mm were placed scutellum side down on sterile fiber glass filter paper in a Petri dish. 300 μL of DBC6 liquid medium with 100 mg/L cefotaxime was added to the filter paper to prevent over drying. Plates were wrapped with Parafilm and checked for expression of DsRed before desiccation in order to compare expression after desiccation. Plates were moved into a sterile laminar hood unwrapped and let stand for 2-4 days until the embryos appeared darker and shrunken, and were desiccated. Embryos were then placed scutellum side down onto MSA regeneration medium containing 100 mg/L cefotaxime with 10-50 uM glyphosate for selection. Five to 10 days later, DsRed expression is checked in the emerging shoots.

### Example 6

### Natural Desiccation and Excision in Transgenic Mature Corn Seed

Immature embryos of maize inbred PHR03 were transformed with the excision vector AGL1/PHP54353, the expression of DsRed was visually confirmed, and T₀ plantlets were regenerated as described in Example 5. Before moving the T₀ plantlets to soil, the expression of DsRed was again visually confirmed.

### Glyphosate Resistance Confirmation

To confirm that the natural desiccation process that occurs during seed maturation would in fact allow for the excision of DsRed and resistance to glyphosate, seeds collected from T₀ plants crossed with wild-type PHR03 pollen were germinated in soil. By planting seeds straight to soil without any treatments, excision would be a result of natural processes.

Three random events were chosen to be tested by this method. Five mature T₁ seeds each from the following events, PHP54353 T₀ event numbers 6, 7, and 10 were placed in small pots with Metro Mix soil (Sun Gro Horticulture, McFarland, Calif.) with fertilizer and placed in the greenhouse. After plants had germinated and grown to about 12-18 cm (10-12 days after planting), the plants were then sprayed with glyphosate+surfactant at 2× or 4× concentration (1× is equivalent to what is used in the field). Before spraying, all pots were evenly spaced and positioned to ensure that they would receive an even distribution of glyphosate. The distance between the sprayer nozzle and the apical meristem of the plants was approximately 18 inches. Within 10-12 days, it was visibly evident which plants were not affected by the herbicide and which plants had been severely damaged.

The results of the spray test are presented in Table 10. From visible spray test results, all wild-type PHR03 plants had been severely damaged, as predicted. It was also clear that 2 out of 4 plants from event number 6 had no signs of damage and continued to grow at a normal rate having not lost any leaf tissue. However, all 5 plants from event number 7 did show damage equivalent to that of the wild-type PHR03 plants, which was not expected. All 4 plants from event number 10 also showed damage equivalent to that of the wild-type PHR03 plants. When the T₀ plants were analyzed for the presence of the DsRED and GLYAT genes, it was discovered that event number 10 did not have the DsRED gene and although the T₀ plant had the GLYAT gene, presumably GLYAT was not expressed because it was not operably linked to a promoter (see Table 10). In event number 13, 3 out of 5 plants showed damage and 2 out of 5 plants were tolerant.

TABLE 10
Glyphosate Spray Test on Plants Germinated from T₁
Mature Corn Seed

| | DS-RED2INT | GLYAT | Glyphosate Spray |
|---|---|---|---|
| Lab event # | QPCR of T₀ | QPCR of T₀ | Test |
| 6 | + | + | 2/4 plants damaged; |
| | | | 2/4 plants tolerant |
| 7 | + | + | 5/5 plants damaged |
| 10 | − | + | 4/4 plants damaged |
| 13 | + | + | 3/5 plants damaged; |
| | | | 2/5 plants tolerant |
| Wild-type | − | − | 4/4 plants damaged |

### Example 7

### Tobacco Excision Induction and Plant Regeneration from Transformed Tissue Events

### Tobacco Transformation

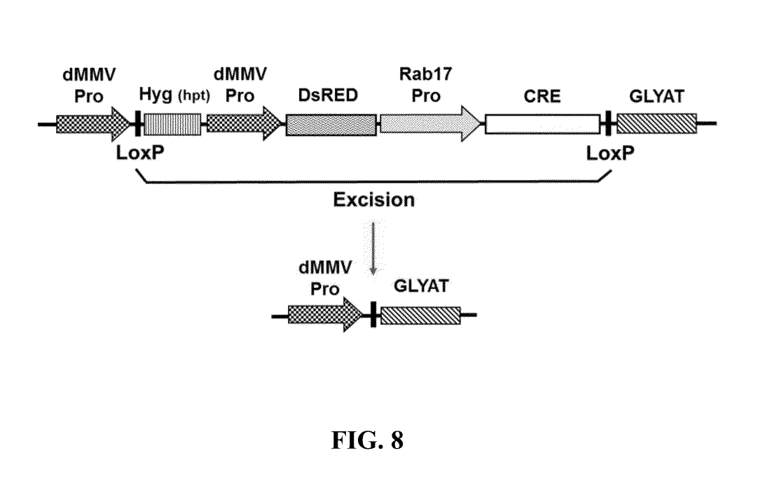

Young leaves are harvested from in vitro-cultured tobacco plants and cut into 0.5-1 cm size as an Agrobacterium infection target. AGL1/PHP55062 (a standard excision vector, FIG. 8) is used for transformation. Transgenic tobacco (cv. Petite havana) plants are generated following the leaf disc method described by Horsch et al. (1985) Science 227:1229-1231, which is herein incorporated by reference in its entirety, and 50 mg/L hygromycin B was used for selection.

Excision of LoxP Cassette by Desiccation and Plant Regeneration with Glyphosate Selection

Tobacco desiccation experiments are conducted to induce excision from transformed tissue events and transformed plants are regenerated. Once tissue from each event having visual marker expression has reached a sufficient size when grown on selection medium with hygromycin, desiccation experiments can be conducted. Tissues (0.3-0.5 mm in diameter) are sliced and transferred to an empty 60 mm×25 mm Petri dish containing a piece of sterilized glass filter paper (VWR Glass Microfibre filter, 691). The Petri dish is covered and placed in a container with a tight-seal cover. An open Petri dish with 15 mL of sterilized water is placed in the container. The container is placed in a dark culture room at 28° C. After 2-3 days of desiccation treatment, the tissues are either directly transferred to regeneration medium or selection medium with antibiotics and 20-50 uM glyphosate using Phytagel as a gelling agent for 2-3 weeks with sealed plates for proliferation and regeneration. The tissues are transferred to regeneration medium with antibiotics and 20-50 uM glyphosate for another 2-4 weeks to generate shoots. Plates are placed in higher intensity light at 26-28° C. When shoots are strong enough, single plantlets are separated and transferred to soil. Leaf samples are collected for qPCR analysis.

### Example 8

### Tobacco Excision Induction and Plant Regeneration from Desiccated T₁ Immature Seeds

T₁ immature seeds from transgenic tobacco plants are isolated, sterilized with 15% Clorox+2 drops of Tween 20 and rinsed with autoclaved water 3 times. Sterilized immature seeds are placed on sterile fiber glass filter paper in a Petri dish. The Petri dish is covered and moved into a sterile laminar hood unwrapped and incubated for 1-2 days until the seeds are desiccated. Desiccated immature seeds are then placed onto regeneration medium containing 100 mg/L cefotaxime and with 20-50 μM glyphosate for selection. One to 2 weeks later, DsRed expression is checked in the emerging shoots. Immature seeds that have been properly desiccated have very weak or no DsRed expression as the gene is excised via the LoxP sites. Both transgenic and nontransgenic seeds without desiccation treatment will germinate well on glyphosate-free medium while germination will be completely inhibited for both of them on 20-50 μM glyphosate. Immature seeds that successfully underwent gene excision by desiccation will have glyphosate resistance and regenerate on medium containing 20-50 μM glyphosate.

Healthy plantlets are transferred to regeneration medium in Phytatrays containing 100 mg/L cefotaxime and 20-50 μM glyphosate for further selection and growth.

### Example 9

### Natural Desiccation and Excision in Transgenic Mature Tobacco Seeds

Mature seed sterilization, Selection/Regeneration:

T₁ mature tobacco seed transformed with AGL1/PHP55062 are sterilized with 20% Clorox+2 drops Tween 20 and rinsed with autoclaved water 3 times. Sterilized seeds are then transferred to regeneration medium containing 100 mg/L cefotaxime with 20-50 μM glyphosate for selection. After 5-10 days, DsRed expression is checked in the emerging shoots. Seeds that have been excised will no longer have DsRed expression as the gene is cleaved via the Lox P sites. Those seeds that are successfully excised of DsRed will have glyphosate resistance and regenerate on medium containing glyphosate. Once seeds have healthy shoot and root formation, the plantlets are moved to soil or another regeneration medium containing 100 mg/L cefotaxime in Phytatrays with 20 or 50 μM glyphosate for further selection and growth.

### Sowing Dry Tobacco T₁ Seeds Straight to Soil and Glyphosate Resistance Confirmation:

To confirm that the natural desiccation process that occurs during seed maturation would in fact allow for the excision of DsRed and resistance to glyphosate, seeds collected from T₀ tobacco plants are germinated in soil. By planting seeds straight to soil without any treatments, excision would truly be a result of natural processes. After plants have germinated and grown to about 10-15 cm, the plants are sprayed with glyphosate+surfactant at 2× or 4× concentration (1× is equivalent to what is used in the field). Within 10-12 days, it is visibly evident which plants are not affected by the herbicide and which plants are severely damaged.

### Example 10

### Soybean Excision Induction and Plant Regeneration from Transformed Tissue Events

### Soybean Transformation:

Soybean (cv. Jack) mature seeds are sterilized and sliced into half longitudinally and half-seeds are used as an Agrobacterium infection target. Agrobacterium strain AGL1 containing the PHP55062 vector (a standard excision vector, FIG. 8) is used for transformation. Alternatively, soybean embryogenic suspension cultures are transformed with the PHP55062 vector via Agrobacterium-mediated transformation as described herein or by the method of particle gun bombardment (Klein et al. (1987) Nature, 327:70, which is herein incorporated by reference in it entirety).

Transgenic soybean plants are generated following the method described in U.S. Pat. No. 7,473,822, which is herein incorporated by reference in its entirety, and 5 to 30 mg/L hygromycin B is used for selection.

Excision of LoxP Cassette by Desiccation and Plant Regeneration with Glyphosate Selection:

Soybean desiccation experiments are conducted to induce excision from transformed tissue events and transformed plants are regenerated. Once tissue from each event having visual marker expression has reached a sufficient size when grown on selection medium with hygromycin, desiccation experiments can be conducted. Tissues (0.3-0.5 mm in diameter) are sliced and transferred to an empty 60 mm×25 mm Petri dish containing a piece of sterilized glass filter paper (VWR Glass Microfibre filter, 691). The Petri dish is covered and placed in a container with a tight-seal cover. An open Petri dish with 15 mL of sterilized water is placed in the container. The container is placed in a dark culture room at 28° C. After 2-3 days of desiccation treatment, the tissues are either directly transferred to regeneration medium with antibiotics and 20-50 μM glyphosate using Phytagel as a gelling agent for 2-3 weeks with sealed plates for proliferation and regeneration. The tissues are transferred to regeneration medium with antibiotics and 20-50 μM glyphosate for another 2-4 weeks to generate shoots. Plates are placed in higher intensity light at 26-28° C. When shoots are strong enough, single plantlets are separated and transferred to soil. Leaf samples were collected for qPCR analysis.

### Example 11

### Soybean Excision Induction and Plant Regeneration from Desiccated T₁ Immature Seeds

T₁ immature pods from transgenic soybean plants are harvested, sterilized with 15% Clorox+2 drops of Tween 20 and rinsed with autoclaved water 3 times. Immature seeds are isolated from sterilized pods and placed on sterile fiber glass filter paper in a Petri dish. The Petri dish is covered and moved into a sterile laminar hood unwrapped and incubated for 1-2 days until the seeds are desiccated. Desiccated immature seeds are then placed onto regeneration medium containing 100 mg/L cefotaxime and with 20-50 μM glyphosate for selection. One to 2 weeks later, DsRed expression is checked in the emerging shoots. Immature seeds that have been properly desiccated will have very weak or no DsRed expression as the gene is excised via the LoxP sites. Both transgenic and nontransgenic seeds without desiccation treatment will germinate well on glyphosate-free medium while germination will be completely inhibited for both of them on 20-50 μM glyphosate. Immature seeds that successfully underwent gene excision by desiccation will have glyphosate resistance and regenerate on medium containing 20-50 μM glyphosate.

Healthy plantlets are transferred to regeneration medium in Phytatrays containing 100 mg/L cefotaxime and 20-50 uM glyphosate for further selection and growth.

### Example 12

### Natural Desiccation and Excision of Transgenic Mature Soybean Seeds

### Mature Seed Sterilization, Selection/Regeneration:

T₁ mature soybean seed transformed with AGL1/PHP55062 are sterilized with 20% Clorox+2 drops Tween 20 and rinsed with autoclaved water 3 times. Sterilized seeds are then transferred to regeneration medium containing 100 mg/L cefotaxime with 20-50 μM glyphosate for selection. After 5-10 days, DsRed expression is checked in the emerging shoots. Seeds that have been excised will no longer have DsRed expression as the gene is cleaved via the Lox P sites. Those seeds that are successfully excised of DsRed will have glyphosate resistance and regenerate on medium containing glyphosate. Once seeds have healthy shoot and root formation, the plantlets are moved to soil or another regeneration medium containing 100 mg/L cefotaxime in Phytatrays with 20 or 50 μM glyphosate for further selection and growth.

### Sowing Dry Soybean T₁ Seeds Straight to Soil and Glyphosate Resistance Confirmation:

To confirm that the natural desiccation process that occurs during seed maturation would in fact allow for the excision of DsRed and resistance to glyphosate, seeds collected from Tₒ soybean plants are germinated in soil. By planting seeds straight to soil without any treatments, excision would be a result of truly natural processes. After plants have germinated and grown to about 10-15 cm, the plants are sprayed with glyphosate+surfactant at 2× or 4× concentration (1× is equivalent to what is used in the field). Within 10 days, it is visibly evident which plants are not affected by the herbicide and which plants are severely damaged.

### Example 13

### Agrobacterium-Mediated Transformation of Wheat Using Immature Embryos (IEs) with Standard and Sand Treatments

### Preparation of Agrobacterium Suspension:

Agrobacterium tumefaciens harboring vector of interest was streaked from a −80° frozen aliquot onto solid LB medium containing selection (kanamycin or spectinomycin). The Agrobacterium was cultured on the LB plate at 21° C. in the dark for 2-3 days. A single colony was selected from the master plate and was streaked onto an 810D medium plate containing selection and it was incubated at 28° C. in the dark overnight. A sterile spatula was used to collect Agrobacterium cells from the solid medium and cells were suspended in 5 mL wheat infection medium (WI4) with 400 uM acetosyringone (As) (Table 1). The OD of the suspension was adjusted to 0.1 at 600 nm using the same medium.

### Wheat Immature Embryo Transformation:

### Material Preparation, Sterilization and Sand Treatment

4-5 spikes were collected, containing immature seeds with 1.5-2.5 mm embryos. Immature seeds/wheat grains were then isolated from the spike by pulling downwards on the awn and removing both sets of bracts (the lemma and palea). Wheat grains were surface-sterilized for 15 min in 20% (v/v) bleach (5.25% sodium hypochlorite) plus 1 drop of Tween 20, and then they were washed in sterile water 2-3 times. Immature embryos (IEs) were isolated from the wheat grains and were placed in 1.5 ml of the WI4 medium into 2 mL micro-centrifuge tubes. Immature embryos were isolated and placed in 1 mL of WI4 medium with 0.25 mL of autoclaved sand. The 2 mL microcentrifuge tubes containing the immature embryos were centrifuged at 6k for 30 seconds, vortexed at 4.5, 5 or 6 for 10 seconds, and then centrifuged at 6k for 30 seconds. Embryos were let stood for 20 minutes.

Embryo Treatments with Sand and Infection

WI4 medium was drawn off, and 1.0 ml of Agrobacterium suspension was added to the 2 mL microcentrifuge tubes containing the immature embryos. Embryos were let to stand for 20 minutes. The suspension of Agrobacterium and immature embryos was poured onto wheat co-cultivation medium, WC21 (Table 2). Any embryos left in the tube were transferred to the plate using a sterile spatula. The immature embryos were placed embryo axis side down on the media, and it was ensured that the embryos were immersed in the solution. The plate was sealed with Parafilm tape and incubated in the dark at 25° C. for 3 days of co-cultivation.

Media Scheme and selection

After 3 days of co-cultivation immature embryos were transferred embryo axis side down to DBC4 green tissue (GT) induction medium containing 100 mg/L cefotaxime (PhytoTechnology Lab., Shawnee Mission, Kans.) (Table 3). All embryos were then incubated at 26-28° C. in dim light for two weeks, then were transferred to DBC6 tissue (GT) induction medium containing 100 mg/L cefotaxime for another two weeks (Table 4). Regenerable sectors appear 3-4 weeks after transformation and will be ready for regeneration after being isolated. Regenerable sectors were cut from the non-transformed tissues and placed on regeneration media MSA with 100 mg/L cefotaxime (Table 5). Sectors on MSA medium should be placed in bright light for 1.5-2 weeks or until roots and elongated shoots have formed. After sectors have developed into small plantlets they were transferred to Phyta trays until plantlets are ready to be transferred to soil. During each transfer plantlets were checked for marker gene expression and any non-expressing or chimeric tissues were removed.

TABLE 11
Liquid Wheat Infection Medium

| WI4 | | |
|---|---|---|
| DI water | 1000 | mL |
| MS salt + Vitamins | 4.43 | g |
| Maltose | 30 | g |
| Glucose | 10 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| Adjust PH to 5.8 with KOH | | |
| Post sterilization | | |
| Acetosyringone (1 M) | 400 | μl |

TABLE 12
Wheat Co-cultivation Medium

| WC21 | | |
|---|---|---|
| DI water | 1000 | mL |
| MS salt + Vitamins | 4.43 | g |
| Maltose | 30 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| 50X CuSO4 (0.1 M) | 49 | μl |
| Adjust PH to 5.8 with KOH | | |
| Add 3.5 g/L of Phytagel | | |
| Post sterilization | | |
| Acetosyringone (1 M) | 400 | μl |

TABLE 13
DBC 4 medium
DBC4

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1 M) | 49 | μL |
| 2,4-D (0.5 mg/mL) | 2 | mL |
| BAP | 1 | mL |
| Adjust PH to 5.8 with KOH | | |
| Add 3.5 g/L of Phytagel | | |
| Post sterilization | | |
| Cefotaxime (100 mg/ml) | 1 | ml |

TABLE 14
DBC 6 medium
DBC6

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 | 10 | mL |
| mg/mL) | | |
| 50X CuSO4 (0.1 M) | 49 | μL |
| 2,4-D (0.5 mg/mL) | 1 | mL |
| BAP | 2 | mL |
| Adjust PH to 5.8 with KOH | | |
| Add 3.5 g/L of Phytagel | | |
| Post sterilization | | |
| Cefotaxime (100 mg/ml) | 1 | ml |

TABLE 15
Regeneration MSA medium
MSA

| dd H20 | 1000 | mL |
|---|---|---|
| MS salt + Vitamins(M519) | 4.43 | g |
| Sucorse | 20 | g |
| Myo-Inositol | 1 | g |
| Adjust PH to 5.8 with KOH | | |
| Add 3.5 g/L of Phytagel | | |
| Post sterilization | | |
| Cefotaxime (100 mg/ml) | 1 | ml |

Wheat Agrobacterium-mediated transformation using immature embryos were conducted with standard treatments and sand treatments to compare the transformation frequencies at T0 plant level.

Table 16 shows the transformation frequencies at T0 plant level (T0) for transformation experiments with standard and sand treatments using Standard vector for Pioneer elite spring wheat variety SBC0456D; the binary vectors are difficult constructs for transformation because the visual marker is driven by weal promoter for selection. All experiments were performed with 4.5-6 vortex speed for both standard and sand treatments. Data showed that TO frequencies ranged from 0% to 1.2% for standard treatments. For sand treatments, TO frequencies ranged from 5.9% to 6.8%. Results indicated that experiments conducted with sand treatments had higher transformation frequencies comparing to standard treatments.

TABLE 16
Agrobacterium-mediated transformation of immature embryos using standard
vector with standard and sand treatments

| | | 0.25 mL | | | | |
|---|---|---|---|---|---|---|
| | Standard | sand | | 0.25 mL | | 0.25 mL |
| | Vortex at | Vortex at | Standard | sand | Standard | sand |
| Treatments | 4.5 | 4.5 | Vortex at 5 | Vortex at 5 | Vortex at 6 | Vortex at 6 |
| Transformation | 0% (0/52) | 5.9% (3/51) | 0% (0/46) | 18.6% | 0% (0/48) | 13.3% |
| Frequency | | | | (8/43) | | (6/45) |
| (T0) | | | 0% (0/54) | 3.7% (2/54) | 0% (0/66) | 1.4% (1/72) |
| | | | 2.8% (2/71) | 1.5% (1/65) | | |
| Average | 0% (0/52) | 5.9% (3/51) | 1.2% | 6.8% | 0% (0/114) | 6.0% |
| | | | (2/171) | (11/162) | | (7/117) |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A wheat plant cell comprising a polynucleotide construct comprising:
a) an excision cassette, comprising an expression cassette A (EC_{A}) comprising:
i) a coding polynucleotide A (CP_{A}) encoding a site specific recombinase; and
ii) an inducible promoter A (P_{A}) operably linked to the CP_{A};
b) a first and a second recombination site flanking the excision cassette;
c) a coding polynucleotide B (CP_{B}) encoding a herbicide tolerance polypeptide; and
d) a promoter B (P_{B}), wherein the P_{B} is operably linked to the CP_{B} after excision of the excision cassette.

2. The wheat plant cell of claim 1, wherein the inducible promoter P_{A} is selected from the group consisting of a stress-inducible promoter and a chemical-inducible promoter.

3. The wheat plant cell of claim 2, wherein said chemical-inducible promoter comprises a promoter comprising a tet operator.

4. The wheat plant cell of claim 3, wherein said polynucleotide construct further comprises a coding polynucleotide F (CP_{F}) encoding a sulfonylurea-responsive transcriptional repressor protein, wherein said CP_{F} is operably linked to a promoter active in a plant cell.

5. The wheat plant cell of claim 2, wherein the stress-inducible promoter can be induced in response to cold, drought, high salinity, desiccation, or a combination thereof.

6. The wheat plant cell of claim 2, wherein the stress-inducible promoter comprises a nucleotide sequence selected from the group consisting of:
a) the nucleotide sequence having the sequence set forth in SEQ ID NO: 18;
b) a nucleotide sequence having at least 70% sequence identity to the sequence set forth in SEQ ID NO: 18;
c) a nucleotide sequence comprising at least 50 contiguous nucleotides of the sequence set forth in SEQ ID NO: 18;
d) the nucleotide sequence set forth in nucleotides 291-430 of SEQ ID NO: 18; and
e) a nucleotide sequence having at least 70% sequence identity to the sequence set forth in nucleotides 291-430 of SEQ ID NO: 18.

7. The wheat plant cell of claim 1, wherein the inducible promoter P_{A} is a vernalization promoter.

8. The wheat plant cell of claim 1, wherein the P_{B} is a constitutive promoter.

9. The wheat plant cell of claim 8, wherein the P_{B} is selected from the group consisting of a ubiquitin promoter, an oleosin promoter, an actin promoter, and a Mirabilis mosaic virus (MMV) promoter.

10. The wheat plant cell of claim 1, wherein the excision cassette further comprises a coding polynucleotide C(CP_{C}) encoding a selectable marker, wherein the CPc is operably linked to a promoter active in a plant cell.

11. The wheat plant cell of claim 10, wherein the CP_{C} is operably linked to P_{B} prior to excision of the excision cassette.

12. The wheat plant cell of claim 10, wherein the excision cassette further comprises a promoter C(P_{C}) operably linked to the CP_{C}.

13. The wheat plant cell of claim 12, wherein the P_{C} is a constitutive promoter.

14. The wheat plant cell of claim 10, wherein the selectable marker is selected from the group consisting of a fluorescent protein, an antibiotic resistance polypeptide, a herbicide tolerance polypeptide, and a metabolic enzyme.

15. The wheat plant cell of claim 1, wherein the herbicide tolerance polypeptide encoded by CP_{B} comprises a glyphosate-N-acetyltransferase (GLYAT) polypeptide or an ALS inhibitor-tolerance polypeptide.

16. The wheat plant cell of claim 15, wherein said ALS inhibitor-tolerance polypeptide comprises the highly resistant ALS (HRA) mutation of acetolactate synthase.

17. The wheat plant cell of claim 1, wherein the excision cassette further comprises a coding polynucleotide D (CP_{D}) encoding a cell proliferation factor operably linked to a promoter active in a plant cell.

18. The wheat plant cell of claim 17, wherein the cell proliferation factor is a selected from a WUSCHEL polypeptide and a babyboom polypeptide.

19. The wheat plant cell of claim 18, wherein the babyboom polypeptide comprises at least two AP2 domains and at least one of the following amino acid sequences:
a) the amino acid sequence set forth in SEQ ID NO: 67 or an amino acid sequence that differs from the amino acid sequence set forth in SEQ ID NO: 67 by one amino acid; and
b) the amino acid sequence set forth in SEQ ID NO: 68 or an amino acid sequence that differs from the amino acid sequence set forth in SEQ ID NO: 68 by one amino acid.

20. The wheat plant cell of claim 18, wherein the CP_{D} has a nucleotide sequence selected from the group consisting of:
a) the nucleotide sequence set forth in SEQ ID NO: 55, 57, 58, 60, 74, 76, 78, 80, 82, 84, 86, 87, 88, 90, 92, 94, 96, 98, 99, or 101;
b) a nucleotide sequence having at least 70% sequence identity to SEQ ID NO: 55, 57, 58, 60, 74, 76, 78, 80, 82, 84, 86, 87, 88, 90, 92, 94, 96, 98, 99, or 101;
c) a nucleotide sequence encoding a polypeptide having the amino acid sequence set forth in SEQ ID NO: 56, 59, 75, 77, 79, 81, 83, 85, 89, 91, 93, 95, 97, 100, or 102; and
d) a nucleotide sequence encoding a polypeptide having an amino acid sequence having at least 70% sequence identity to the amino acid sequence set forth in SEQ ID NO: 56, 59, 75, 77, 79, 81, 83, 85, 89, 91, 93, 95, 97, 100, or 102.

21. The wheat plant cell of claim 18, wherein the polynucleotide encoding a WUSCHEL polypeptide has a nucleotide sequence selected from the group consisting of:
a) the nucleotide sequence set forth in SEQ ID NO: 103, 105, 107, or 109; and
b) a nucleotide sequence having at least 70% sequence identity to SEQ ID NO: 103, 105, 107, or 109;
c) a nucleotide sequence encoding a polypeptide having the amino acid sequence set forth in SEQ ID NO: 104, 106, 108, or 110; and
d) a nucleotide sequence encoding a polypeptide having an amino acid sequence having at least 70% sequence identity to SEQ ID NO: 104, 106, 108, or 110.

22. The wheat plant cell of claim 21, wherein the polynucleotide encoding a WUSCHEL polypeptide is operably linked to a maize In2-2 promoter or a nopaline synthase promoter.

23. The wheat plant cell of claim 17, wherein the excision cassette further comprises a promoter D (P_{D}) operably linked to the CP_{D}.

24. The wheat plant cell of claim 23, wherein the P_{D} is a constitutive promoter.

25. The wheat plant cell of claim 24, wherein the P_{D} is a ubiquitin promoter or an oleosin promoter.

26. The wheat plant cell of claim 17, wherein the excision cassette comprises at least a first coding polynucleotide D (CP_{D1}) encoding a babyboom polypeptide and a second coding polynucleotide D (CP_{D2}) encoding a WUSCHEL polypeptide.

27. The wheat plant cell of claim 1, wherein the polynucleotide construct further comprises a coding polynucleotide E (CP_{E}) encoding a polypeptide of interest, wherein the CP_{E} is operably linked to a promoter active in a plant cell.

28. The wheat plant cell of claim 27, wherein the CP_{E} is outside of the first and a second recombination sites flanking the excision cassette.

29. The wheat plant cell of claim 1, wherein said wheat plant cell is a cell of a winter wheat.

30. The wheat plant cell of claim 29, wherein said wheat plant cell is a cell of Triticum aestivum or Triticum monococcum.

31. A wheat plant or wheat plant part comprising the wheat plant cell of claim 1.

32. The wheat plant or wheat plant part of claim 31, wherein the plant or plant part is recalcitrant to transformation.

33. The wheat plant or wheat plant part of claim 31, wherein the plant part is a seed.

34. A method for regulating the expression of a herbicide tolerance polynucleotide, wherein the method comprises:
a) providing the wheat plant cell of claim 1; and,
b) inducing the expression of the site-specific recombinase, thereby excising the excision cassette from the polynucleotide construct and expressing the herbicide tolerance polynucleotide.

35. A method for selecting a herbicide tolerant wheat plant cell, the method comprising the steps of:
A) providing a population of wheat plant cells, wherein at least one wheat plant cell in the population is a wheat plant cell according to claim 1;
B) inducing the expression of the site-specific recombinase; and
C) contacting the population of plant cells with a herbicide to which the herbicide tolerance polypeptide confers tolerance, thereby selecting for a plant cell having tolerance to the herbicide.

36. The method of claim 35, wherein the method further comprises introducing the polynucleotide construct into the at least one wheat plant cell before step A).

37. The method of claim 35, wherein the inducible promoter A (P_{A}) is induced in response to cold, drought, desiccation, high salinity or a combination thereof.

38. The method of claim 35, wherein the inducing comprises desiccating the population of wheat plant cells.

39. The method of claim 38, wherein the desiccating occurs during the maturation of an immature seed.

40. The method of claim 35, wherein the excision cassette further comprises a coding polynucleotide C(CP_{C}), wherein the CP_{C} encodes a selectable marker operably linked to a promoter, and wherein the method further comprises a selection step prior to step B), wherein those wheat plant cells within the population of wheat plant cells that comprise the selectable marker are identified and wherein these selected wheat plant cells comprise the population of wheat plant cells that are induced in step B).

41. A method for increasing the transformation efficiency of a wheat plant tissue, the method comprising the steps of:
a) providing a population of wheat plant cells, wherein at least one wheat plant cell in the population is a wheat plant cell according to claim 1;
b) culturing the population of wheat plant cells in the absence of a herbicide to which the herbicide tolerance polypeptide confers herbicide resistance for a period of time sufficient for the population of wheat plant cells to proliferate;
c) inducing the expression of the site-specific recombinase, thereby excising the excision cassette;
d) contacting the population of wheat plant cells from c) with the herbicide to which the herbicide tolerance polypeptide confers tolerance; and
e) selecting for a wheat plant cell having tolerance to the herbicide, wherein the transformation frequency is increased compared to a comparable wheat plant cell not comprising the excision cassette and selected directly by herbicide selection.

42. The method of claim 41, wherein the inducing comprises desiccating the population of wheat plant cells.

43. The method of claim 41, wherein the population of wheat plant cells is cultured in the absence of the herbicide to which the herbicide tolerance polypeptide confers herbicide resistance for about 1 hour to about 6 weeks prior to excision.
